(19) United States
(12) Patent Application Publication
(10) Pub. No.: US 2011/0195848 A1
(43) Pub. Date: Aug. 11, 2011

(54) GENE EXPRESSION AND BREAST CANCER

(76) Inventors: Avtar S. Roopra, Madison, WI (US); Matthew P. Wagoner, Wilmington, DE (US)

(21) Appl. No.: 12/987,910

(22) Filed: Jan. 10, 2011

Related U.S. Application Data
(60) Provisional application No. 61/293,404, filed on Jan. 8, 2010.

(51) Int. Cl. C12Q 1/68; C40B 30/00

(52) U.S. Cl. 506/7; 435/6.14; 435/6.12

(57) ABSTRACT

This invention provides methods and reagents for determining breast cancer patient prognosis and/or diagnosis of tumor aggressiveness, disease-free survival times and reduced patient disease-free survival metrics.

This application claims the priority benefit of U.S. provisional patent application Ser. No. 61/293,404 filed Jan. 8, 2010, the entirety of which is herein incorporated by reference. The sequence listing submitted herewith is incorporated by reference in its entirety.

### FIELD OF THE INVENTION

This invention provides diagnostic methods and reagents for identifying cancer, as well as methods and reagents for making a prognosis of cancer patient survival. More particularly, certain embodiments of the invention provide one or a plurality of differentially-expressed genes associated with cancer, wherein said pluralities comprise what are termed herein “gene signatures.” Gene signatures are used according to methods disclosed herein to identify aggressive breast cancers having poorer patient prognosis and lower post-diagnosis survival than breast cancer not displaying a gene signature of the invention. Particularly advantageous gene signatures comprise LIN28, CELF4 or CELF6, which provide useful biomarkers for aggressive breast cancers. Additional gene signatures for aggressive breast cancers comprise genes observed to be upregulated in such cancers. In other embodiments, the invention provides reagents and methods for identifying dysfunction in patient or cell samples of a gene, REST/NRSF, also related to an aggressive breast cancer phenotype. This invention further provides methods and reagents for detecting tumors that express particular REST/NRSF variants, including in particular REST4, indicative of such aggressive breast cancers and methods for determining patient prognosis for individuals having breast cancer tumors expressing said variants. The invention also provides methods and reagents for detecting elevated miR-124, which is identified herein to be elevated in aggressive breast cancers that are deficient in REST function.

### BACKGROUND OF THE INVENTION

Breast cancer is the most common type of cancer among women in the United States. In 2009, an estimated 192,000 U.S. women were newly-diagnosed with breast cancer. (National Cancer Institute (NCI), 2009, www.cancer.gov/cancertopics/types/breast). One histological parameter used to characterize breast cancer tumors is estrogen receptor alpha (ER) status. Approximately 70% of all breast cancers express ER (i.e., they are termed “ER+”). Patients with ER+ tumors tend to have a better prognosis and greater life expectancies than patients with ER deficient (i.e., ER−) tumors (Cella et al., 2006, Breast Cancer Res Treat 100: 273; Howell, 2006, Rev Recent Clin Trials 1: 207). However, the ER+ patient population is heterogeneous. A portion thereof demonstrates poor outcomes despite tumors exhibiting the same molecular, histological and grade markers as patients with more positive prognoses. This observation illuminates a need in the art for identifying robust, reliable markers and prognostic indicators that can accurately predict patient outcome and/or facilitate selection of appropriate breast cancer treatment regimens.

### Neuron Restrictive Silencing Factor (NRSF)

Neuron restrictive silencing factor (NRSF), also known as REST (RE1 Silencing Transcription Factor), represses transcription of neuronal genes in non-neuronal cells by recruiting chromatin modifiers to a 21 bp element termed neuron restrictive silencing elements (NRSE). REST/NRSF was originally isolated in a screen looking for factors that confer neuron-restricted gene expression upon neuronal genes (Chong et al., 1995, Cell 80: 949; Schoenherr et al., 1995, Science 267: 1360). REST/NRSF was found to function by repressing expression of a number of neuronal genes in non-neuronal tissue by binding to NRSEs found in the regulatory regions of these genes. Subsequently, around 2,000 genes have been found to be direct targets of REST/NRSF in human and mouse genomes (Bruce et al., 2004, Proc Natl Acad Sci USA 101: 10458).

A particular mutation in REST/NRSF was found in several colon cancer samples, and thus REST/NRSF was thought to be a possible tumor suppressor gene in colon cancer (Westbrook et al., 2005, Cell, 121:837-848). Subsequently, it was found that REST/NRSF mRNA expression was lost in roughly one third of the colon and small cell lung cancer samples examined. In mammary cells, reducing REST/NRSF function either by RNAi or the use of dominant negative protein expression promoted malignant transformation of genetically-engineered human mammary epithelial cells (Westbrook et al., 2005, Cell 121: 837-848), suggesting that decreased REST/NRSF mRNA levels could be a possible feature of breast cancer etiology. However, the analysis of numerous patient breast tumor samples showed no decrease in REST mRNA levels.

As set forth above, estrogen receptor positive (ER+) breast cancers are a heterogeneous population of cancers with varying etiologies and clinical outcomes. Although many patients with ER+ breast cancers initially respond well to surgery and ER-targeted therapies (including selective estrogen receptor modulators and aromatase inhibitors), these therapies frequently are not sufficient to prevent disease recurrence or metastasis for all patients with ER+ tumors. Likewise, some populations of ER− breast cancer tumors are less responsive to treatment. Thus, some types of ER+ and ER− breast cancers are particularly aggressive and have very low survival rates. There is a need in the art for reagents and methods for identifying aggressive ER+ tumors, aggressive ER− tumors, and therapy-resistant tumors. Such reagents and methods would aid in early identification of aggressive breast cancers, would facilitate selection of appropriately tailored treatment regimens, and in turn promote improved patient survival rates.

### SUMMARY OF INVENTION

This invention provides reagents and methods for identifying patients with aggressive breast cancer tumors. The reagents and methods of this invention are directed to detecting altered, particularly reduced, expression of functional REST/NRSF protein in breast cancer tumor samples. Specific embodiments of the reagents and methods of the described invention are adapted for detecting alternative splice variants of REST/NRSF. In one embodiment, detecting splice variants that produce loss-of-function REST/NRSF protein variants are included; a non-limiting example of such a splice variant is identified herein as REST4. In additional embodiments, the reagents and methods provided herein detect altered, particularly increased gene expression for a plurality of genes disclosed herein to occur in breast tumor samples, including but not limited to genes set forth in greater detail herein (see Tables 1-4, and 6). Certain embodiments of the invention also provide one or a plurality of genes disclosed herein to exhibit altered expression in breast tumor samples, providing in these embodiments diagnostic gene expression profiles (termed herein “gene signatures”) for identifying aggressive breast cancer tumors. In additional embodiments, the invention provides diagnostic methods using such gene signatures to identify individuals having aggressive breast cancer tumors. In other embodiments, the invention provides prognostic methods using such gene signatures for identifying individuals that are expected to have reduced survival rates, having either estrogen receptor positive (ER+) or estrogen receptor negative (ER−) phenotypes. Certain embodiments of the methods of this invention are adapted to identifying aggressive gene signature-bearing tumors from breast tumors otherwise indistinguishable by conventional markers such as, inter alia, ER expression pattern.

In particular embodiments, the invention provides gene signatures comprising one or a plurality of genes as set forth in Table 1 or Table 6 below. In certain embodiments, gene signatures of the invention comprise at least LIN28. In alternative embodiments, gene signatures comprise at least CELF4, CELF5, or CELF6. In a further embodiment, elevated expression levels for certain miRNAs, and in particular, miR-124 provides a signature for aggressive breast cancer tumors.

As used with methods set forth herein, gene signatures provided by the invention are useful for identifying aggressive subsets of breast cancer tumors, particularly ER+ breast cancer tumors, independently of other existing predictors of poor prognoses, such as tumor grade, size, patient age and HER2 status; as set forth above, these conventional disease status markers are inadequate to reliably identify patients bearing tumors with said capacities for aggressive tumor growth. Patient or cell samples exhibiting gene signatures of this invention have been associated with greatly reduced survival rates as set forth herein below. As provided herein, certain of the genes in a gene signature are upregulated (wherein expression of said gene is higher than in non-tumor breast tissue) to varying degrees in certain breast tumor samples. Upregulation of gene expression in said genes comprising gene signatures of the invention can be detected from breast cancer samples using methods known to the skilled worker, including in non-limiting examples microarray analysis, conventional hybridization-based RNA detection assays, immunoassay and immunohistochemistry (IHC) and protein-directed techniques (such as biochemical activity assays). Additional embodiments of the methods of the invention are provided to detect aggressive breast cancer tumor samples having altered, particularly reduced, expression of functional REST/NRSF. Detection methods for gene signatures can also be used to detect reduced or otherwise altered REST/NRSF expression, including REST4, in breast cancer samples.

In other aspects, the invention provides methods for prognosing breast cancer survival and methods for selecting appropriate drug treatment regimens based on tumor aggressiveness. Identifying gene status and/or aggressiveness of a breast tumor reduces the likelihood that a treatment having a low probability of success will be administered, and enables patients and practitioners to make improved quality-of-life decisions.

The invention also provides kits for performing the methods disclosed herein.

The use of the methods of this invention is beneficial for early detection of reduced prognosis of patient survival using breast cancers tumor samples, regardless of the status of estrogen receptor or other conventional prognostic markers in such tumors. This in turn permits clinical selection of drug therapies better suited to aggressive tumors, promoting improved patient survival rates.

Specific preferred embodiments of the present invention will become evident from the following more detailed description of certain preferred embodiments and the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

This invention can be further appreciated and understood from the following detailed description taken in conjunction with the drawings wherein:

FIGS. 1A-1D are graphs illustrating that REST/NRSF mRNA is not significantly reduced or absent in breast tumors with respect to normal breast tissue. FIG. 1A shows graphs of relative REST/NRSF mRNA levels for two different datasets of breast tumor and normal breast tissue samples. The E-TABM-276 dataset (Normal: n=10, Breast Tumor: n=51) and GDS2250 dataset (Normal: n=7, Breast Tumor: n=40) are shown, wherein mean REST/NRSF mRNA levels in both tumor and normal tissue are illustrated (+/−Standard Deviation). FIG. 1B shows graphs of REST/NRSF mRNA levels for each individual tumor in the E-TABM-276 and GDS2250 datasets represented in FIG. 1A. FIG. 1C is a graph of mean REST/NRSF mRNA levels compared across varying tumor grades (+/−Standard Deviation) in breast tumor dataset GSE5460. FIG. 1D shows graphs of mean REST/NRSF mRNA levels. Levels are substantially unchanged across REST/NRSF negative tumors (RESTless, GDS2250) and REST/NRSF positive tumors (RESTfl tumors, GSE5460) (+/−Standard Error).

FIG. 2A is a photograph of Western blot analysis demonstrating REST/NRSF expression in three REST/NRSF expression knock-down cell lines (HEK, MCF10a, and T47D). REST/NRSF expression was knocked down using lentiviral delivery of shRNA specific for REST/NRSF (shREST) or negative control non-targeting shRNA (shControl). Positive controls for relative protein levels are shown by Actin in bottom panels. FIG. 2B is a Venn diagram illustrating the commonality of genes that were up-regulated at least 2-fold in the REST/NRSF knock-down cell lines. Twenty-four genes were in common between all three cell lines. (See Table 1).

FIG. 3 illustrates microarray results for gene expression from breast cancer tumor samples. mRNA expression levels of cellular genes (the positions of which are identified on the righthand side of the array) in breast cancer tumors (identified across the top border of the array) were assessed. Increased gene expression is shown in red (clustered in the center of the microarray).

FIG. 4 illustrates microarray results for gene expression from small cell lung cancer tumor samples and cell lines including the H69 SCLC cell line that is known to show high levels of aberrant REST splicing. mRNA expression for a number of housekeeping genes and genes with REST/NRSF-regulated expression are shown, wherein red indicated increased gene expression (see arrow).

FIG. 5 illustrates microarray results for gene expression from breast cancer tumor samples from the U.S. and Sweden, where the X axis represents individual tumors and the Y axis represents specific genes. Two breast cancer microarray databases were interrogated for the presence of the REST/NRSF gene signature and tumors with REST/NRSF dysfunction identified, wherein increased gene expression is shown in red (clustered in the lower lefthand corner of the U.S. array and approximately the middle of the Swedish sample array). Approximately 5% of breasts cancer tumors displayed the REST/NRSF gene signature.

FIG. 6 illustrates microarray results for gene expression from normal and stromal breast tissue. Cluster diagram compares the expression levels of the REST/NRSF gene signature genes across 66 samples of normal breast tissue, taken either as normal breast tissue from mammaplasty or as stromal tissue adjacent to tumor (GSE4823). No enrichment in REST/NRSF target genes was noted in either normal or stromal tissue.

FIG. 7 is a graph of disease-free survival of ER+ breast cancer patients, wherein patients positive for the REST/NRSF gene signature exhibited reduced survival rates compared to patients negative for the gene signature.

FIG. 8A illustrates microarray results for gene expression from 129 breast cancer tumors (GSE5460) interrogated with the 24-gene REST/NRSF gene signature shown in Table 1. Five tumors showed a concerted overexpression of REST/NRSF target genes, suggesting a loss of REST/NRSF repression. FIG. 8B illustrates microarray results for gene expression in RESTless or RESTfl tumors. Expression of genes was significantly upregulated in RESTless tumors (p<10⁻⁷), shown; >85% of these genes are either known or putative REST/NRSF target genes. Arrows indicate tumors from which RNA was available for further analysis. FIG. 8C is a panel of graphs demonstrating Gene Set Enrichment Analysis of breast tumor dataset GSE5460. The graphs illustrate increased expression of REST/NRSF target genes in RESTless tumors using three separate sets of experimentally defined REST/NRSF target genes. The first graph shows that a gene set comprised of 24 genes (termed herein the “24 REST/NRSF gene signature”) that was consistently upregulated at least two-fold (see Table 1) upon experimentally-induced REST/NRSF knockdown in MCF10a, HEK-293 and T47D cell lines was enriched in RESTless tumor samples. The second graph shows that that genes upregulated at least two-fold upon REST/NRSF knockdown across the average of all three cell lines was also enriched in RESTless tumors. The third graph shows the results of this same analysis for a “REST ChIPSeq” gene list (that is populated by genes identified as being bound by REST/NRSF in Jurkat T-cells using ChIPSeq) was enriched in RESTless tumors (Johnson et al., 1997, Science, 326:1497-1502).

FIG. 9A is a photograph of agarose gel electrophoresis of the results from an RT-PCR analysis for full-length REST/NRSF and truncated REST4 splice variants. Tumors positive in microarray assay for the REST/NRSF gene signature were assayed, wherein RNA from two gene signature-bearing tumors (GS1 and GS2) and from a control tumor negative for the REST/NRSF gene signature were subjected to RT-PCR using primers flanking the alternative exon splice site of wildtype and splice variant forms of REST/NRSF. The Figure shows that GS1 and GS2 expressed REST4 splice variant whereas the control tumor expressed full-length REST/NRSF. The lane labeled (--) represents sham amplification with no input RNA. FIG. 9B illustrates full length REST/NRSF and the REST4 alternatively spliced product. Primer sets utilized for quantitative real-time RT- PCR are shown.

FIG. 9B illustrates full length REST/NRSF and the REST4 alternatively spliced product. Primer sets utilized for quantitative real-time RT-PCR are shown.

FIG. 10A is a photograph of an agarose gel illustrating RT-PCR results for REST4 and wild-type REST expression levels. RNA from nine breast tumors was isolated and designated as GSM124998, GSM125004, GSM125011, GSM125015, GSM125019, GSM125027, GSM125050, GSM125080 and GSM125088. RNA was reverse-transcribed and PCR amplified with primers flanking the REST/NRSF alternative intron/exon junction (REST primer set). In FIG. 10B, selective PCR amplification of REST4 from tumor samples (using primers that target the REST4 50 bp exon (REST4 primer set)) demonstrated the presence of REST4 in the RESTless tumors, but not in any of the REST/NRSF competent tumors.

FIG. 11 is a graph of REST4 mRNA relative to Actin. Analysis of REST4 levels in nine tumors represented in the microarray dataset GSE5460 is shown. REST4 mRNA was detected in RESTless, but not RESTfl tumors after 35 cycles of amplification.

FIGS. 12A-12D is a panel of photographs showing immunohistochemically-labeled antibody treatment of REST/NRSF positive breast tissue and RESTless tumors. Paraffin-embedded breast tumor sections were immunohistochemically labeled with an antibody to the C-terminus of REST. FIG. 12A is a photograph of breast tumor that showed strong nuclear staining for the C-terminus of REST. FIG. 12B is a photograph of a different breast tumor stained for REST C-terminus that showed no staining in the nucleus or cytoplasm, indicating a lack of full length REST protein. The significance of these findings is that most, if not all of the known functions of REST involve its localization to the nucleus. Accordingly, cytoplasmic staining in the absence of nuclear staining was also considered to be RESTless. FIGS. 12C and 12D are photographs showing functional loss of REST/NRSF as indicated by the appearance of chromogranin-A, a REST/NRSF target gene, in the RESTless tumors of 12D. Samples that stained negative for REST/NRSF showed a statistically significant enrichment in staining for the REST target chromogranin-A (CHGA), consistent with a loss of REST/NRSF repression. RESTless tumors accounted for 80% of all ectopic chromogranin A staining in the breast (p<0.001). Inset image is enlarged 2× to show detail.

FIG. 13 is a panel of graphs illustrating that a significantly poorer prognosis was observed for patients with REST/NRSF negative (RESTless) tumors. Patients with REST/NRSF negative breast tumors showed significantly decreased disease-free survival time (p=0.007, n=182), and increased incidence of relapse (p=0.054, n=182), particularly in the first three years post-diagnosis.

FIGS. 14A-14D are graphs illustrating that a loss of REST/NRSF increased the aggressiveness of MCF7 tumor growth in nude mouse xenografts. FIG. 12A demonstrates that tumor “take rate” in the mammary fat pads was significantly higher for shREST versus shCon cells (p=0.005). Data is expressed as fraction of injection sites that remained tumor-free. FIG. 14B shows that tumor burden in the mammary fat pads was significantly larger in shREST vs shCon tumors (p=0.005). FIGS. 14C and 14D demonstrate that the tumor take rate (p=0.040) and tumor burden (p=0.037) were greater for shREST than shCon cells when injected subcutaneously into the flanks of athymic nude mice. FIG. 14E is a photograph of a representative bright field microscopy image of a hematoxylin and eosin stained section of an shREST tumor. Arrows indicate muscle fibers incorporated into tumor thereby showing local invasion. Together, these Figures show increased tumorigenesis by REST/NRSF deficient cells.

FIGS. 15A-15E illustrate REST/NRSF regulation of LIN28 expression. FIG. 15A is a panel of graphs showing elevated LIN28 expression levels as determined by quantitative real time RT-PCR in two breast cancer cell lines T47D and MDA-MB-231 that stably express REST-targeted shRNA (i.e., that are REST/NRSF deficient). FIG. 15B is graphs of chromatin immunoprecipitations with an antibody to REST/NRSF showing enrichment of a LIN28 RE1 site 2 kb upstream of the LIN28 promoter. FIG. 15C is a panel of photographs of Western blot analyses of REST, c-Myc, LIN28, beta-actin, and Ras (wherein the antibody used cross-reacted with H, N, and K-Ras) protein from MCF7 cells stably expressing control or REST-targeted shRNA. Representative protein blots are shown, quantitated using a Kodak Image Station 2000R. FIG. 15C includes graphs representing three independent experiments, shown to the right. FIG. 15D is a “Box and Whisker” plot representation of relative LIN28 mRNA levels in the RESTless and RESTfl breast tumors from dataset GSE4922 covering 289 tumors. The lines on the box represent the LIN28 levels in samples from the 75^{th}, 50^{th} and 25^{th} percentiles (top line, middle line, and bottom line, respectively). The whiskers extend to the 90^{th} (top bar) and 10^{th} (bottom bar) percentiles on LIN28 expression in that tumor group, and the ten percent highest and lowest expression values for each individual tumor are expressed as dots outside the whiskers. FIG. 15E illustrates loss of REST/NRSF inhibition of LIN28 is sufficient to account for focus formation of MCF7 cells. Stable expression of shRNA against REST, but not non-targeting control shRNA, induced spontaneous, subconfluent focus formation in MCF7 breast cancer cells. Top left: quantification of spontaneous foci using REST shRNA and a control non-targeting shRNA. Top right: sample foci. Expression of another REST shRNA in a LIN28^{WT} MCF7 cell line also induced spontaneous foci formation. Expression of REST shRNA in LIN28^{low} MCF7 cells expressing shRNA against LIN28, however, did not effectively induce focus formation.

FIG. 16 is a photograph of a Western blot analysis comparing REST/NRSF and LIN28 protein levels in T47D cells expressing REST-targeted shRNA and control. Actin controls are shown at bottom as a loading control.

FIG. 17A-17D demonstrates that REST is a direct transcriptional repressor of LIN28. FIG. 17A is a schematic illustrating the canonical REST binding (RE1) site ˜2 kb upstream of the LIN28 transcriptional start site, which is conserved throughout mammalia. FIG. 17B is a graphical representation of a chromatin immunoprecipitation in MCF7 cells using anti-REST or IgG (sham) antibodies showing that REST bound the LIN28 RE1 site with higher affinity than it bound the RE1 site of the classic REST target gene BDNF. The REST promoter, which does not contain an RE1 site, is shown as a negative control. FIG. 17C is a photograph of a Western blot analysis of LIN28 protein in and REST protein in T47D cells stably expressing a non-targeting control (shCon) or anti-REST shRNA (shREST). FIG. 17D is a photograph of an immunoblot analysis of LIN28, c-Myc and Ras (antibody recognizes H, N and K-Ras) protein in MCF7 cells stably expressing a non-targeting control (shCon) or anti-REST shRNA (shREST). Beta-actin is shown as a loading control in both FIGS. 17C & 17D.

FIGS. 18A and 18B are graphs showing that LIN28 contributed to the migratory phenotype of shREST cells. FIG. 18A shows serum-starved MCF7 cells expressing a control (shCon) or anti-REST (shREST). shRNA were allowed to migrate across a filter containing 8 μm pores towards 10% FBS for 24 hours, and migrated cells were counted. shREST cells are shown to be more migratory than shCon cells (p=0.025). FIG. 18B represents the results of shREST MCF7s further expressing a control (−shLIN28) or anti-LIN28 (+shLIN28) shRNA. Cells were allowed to migrate as in FIG. 18A. shREST cell lost their enhanced migratory phenotype upon knockdown of LIN28 expression.

FIG. 19A-19D is a panel of graphs illustrating that LIN28 contributed to the tumorigenicity of shREST MCF7 cells in mice. shREST-expressing MCF7 cells stably expressing an anti-LIN28 (+shLIN28) or non-targeting control (−shLIN28) shRNA were injected subcutaneously into the flanks or mammary fat pads of athymic nude mice, and tumor take rate was assessed. FIG. 19A shows that tumor take rate in mammary fat pads was decreased upon LIN28 knockdown (p=0.024), with 6/12 control (−shLIN28) and only 1/12 LIN28 knockdown (+shLIN28) injections giving rise to tumors by 100 days post-injection. FIG. 19B shows that tumor burden was decreased when LIN28 is knocked down in shREST MCF7s injected into the mammary fat pads of athymic nude mice (p=0.037); at 100 days post-injection, the volume of control (−shLIN28) tumors was 345 mm³, compared with only 56 mm³ for LIN28 knockdown (+shLIN28) tumors. FIG. 19C shows 100 days post-injection, the overall tumor take rate (at all injection sites) was 42% (10/24) for control but only 12.5% (3/24) for LIN28 knockdown cells (p=0.03). FIG. 14D shows that total tumor burden was decreased in shREST cells expressing an anti-LIN28 shRNA (p=0.02). At 100 days post-injection, the total tumor volume for control (−shLIN28) tumors was 867 mm³, compared with 149 mm³ for LIN28 knockdown (+shLIN28) tumors.

FIG. 20 is a box and whisker plot illustrating that LIN28 mRNA levels were increased in human tumors lacking functional REST. The plot represents LIN28 mRNA levels in 289 RESTless and REST-containing (“RESTfl”) breast tumors from dataset GSE4922 (Ivshina et al., 2006, Cancer Res. 66: 10292-301). The lines on the box represent the 75^{th}, 50^{th} and 25^{th} percentiles; the whiskers represent 90^{th} and 10^{th} percentile of LIN28 expression in each tumor group. The median level of LIN28 expression in RESTless tumors was greater than the 90^{th} percentile for REST-containing tumors.

FIG. 21 are photographs of agarose gel electrophoresis of an RT-PCR analysis for REST4 splice variants. Primers flanking the REST N-exon, which detects both REST and REST4 splice variants, were used to amplify cDNA from HEK-293, MCF7 and T47D cell lines stably expressing shRNA against REST or a non-targeting control sequence. The observed size shift in the REST shRNA cells was indicative of REST4 N-exon inclusion. REST knockdown induced REST4 splicing.

FIG. 22 is a graph of miR-124 expression in MCF7 cells following REST knockdown (Rest shRNA). Mature miR-124 levels are shown as measured by quantitative PCR (Taqman qPCR). REST knockdown in MCF7 cells induces the expression of miR-124, a known REST target, relative to an actin mRNA control. n=6, Wilcoxon rank sum test p<0.05.

FIG. 23 is an illustration of intronic sequences surrounding the REST4 N-exon. The REST4 N-exon is flanked by canonical PTB (polypyrimidine tract binding protein) binding sites. The REST4 N-exon encodes the stop codon responsible for truncating REST to form REST4. The N-exon is flanked on both sides by the canonical PTB binding sequence (UUCU). Consistent with a role for PTB in disrupting exon inclusion, the binding elements are 22 nt 5′ and 42 nt 3′ of the exon-intron junctions. The 5′ PTB binding sequence is contiguous with a polypyrimidine tract, as is often the case in PTB binding elements 5′ of alternative exons.

FIG. 24 is a photograph of a Western blot of protein PTB. Protein lysate from HEK-293 shControl and shREST cells were blotted for PTB, with an actin loading control. HEK-293 shREST cells show diminished PTB protein levels with respect to their control counterparts, indicating the REST knockdown cells express low levels of PTB protein.

FIG. 25A is a photograph of a Western blot of PTB protein in HEK-293 and MCF7 PTB knockdown cell lines. FIG. 25B is a graph representing REST4 levels in the same cells. Stable cell lines expressing shRNA targeting a nontargeting control sequence or PTB were generated. FIG. 25A represents a Western blot confirming PTB knockdown in HEK-293 and MCF7 cells. FIG. 25B shows that PTB knockdown was sufficient to upregulate REST4 expression in both cell lines, as measured by qPCR using REST4 specific primers. Therefore knockdown of PTB induces REST4 splicing in HEK-293 and MCF7 cells. Error bars represent standard error. n=1 for HEK-293 shControls, n=2 for all other samples.

FIG. 26 is a photograph of agarose gel electrophoresis of an RT-PCR analysis for REST and REST4 splice variants on HEK-293 shPTB knockdown cells. Amplification of cDNA from shCon and shPTB HEK-293 cells was performed using primers that detected both REST and REST4 splice variants. Knockdown of PTB was not sufficient to induce the inclusion of the N-exon in a significant fraction of total REST mRNA.

FIG. 27 is a graph illustrating a significance analysis of microarrays identifying genes that were upregulated in MCF7s upon REST knockdown. Expression profiles for MCF7 shCon and shREST cells were assayed by microarray, and the resulting data were analyzed using significance analysis of microarrays (SAM). Gene expression was plotted for each gene with respect to their intensity in shControl and shREST cells. Genes falling along the solid line show equal expression in both cell groups. Genes above the solid line were enriched in shREST cells, below the solid line were enriched in shCon cells. Genes falling outside of the dotted lines had a median false discovery rate <1%, suggesting that their enrichment in either group was unlikely to occur by random chance. 118 mRNAs were significantly upregulated in MCF7 shREST cells (red). The only gene downregulated in shREST cells (green) was REST.

FIG. 28A-28C is a panel of graphs representing REST knockdown induction of CELF4 or CELF6 mRNA upregulation based on microarray data of CELF4 and CELF6 mRNA levels in shControl and shREST HEK-293, T47D and MCF7 cells. FIG. 28A shows that REST knockdown induced CELF6 mRNA in three cell lines that also displayed REST4 splicing upon REST knockdown. FIG. 28B shows that CELF4 mRNA was enriched upon REST knockdown in HEK-293 and MCF7 cells. FIG. 28C confirmed that CELF6 upregulation upon REST knockdown in MCF7 cells was demonstrated by qPCR, confirming what was seen by microarray. CELF6 mRNA level was normalized to beta actin.

FIG. 29 is an illustration of CELF4, CELF5 and CELF6 genes and predicted consensus RE1 sites. Sites for which REST ChIP-Seq data were available have the number of reads for REST and IgG ChIPs graphed underneath each site (Johnson et al., 2007, Science, 316:1497-1502). Coding regions are depicted as black bars, untranslated regions are gray bars.

FIG. 30A-30B is a pair of graphs representing REST chromatin immunoprecipitation in MCF7 cells at CELF4 RE1 sites. Chromatin immunoprecipitation was performed on MCF7 chromatin with non-specific IgG and REST antibodies. FIG. 30A shows that qPCR amplification of the precipitated DNA confirms strong enrichment of REST binding at the double RE1 site in CELF4 intron 7. FIG. 30B shows that enrichment of REST binding is also observed at the first RE1 site in CELF4 intron 1, though the binding is significantly weaker than was observed for the exon 7 RE1 site.

FIG. 31 is a graph illustrating that CELF4 mRNA is elevated in RESTless breast tumors. CELF4 mRNA levels in 129 breast tumors are quantified using six independent probes. Tumors were divided into those that had normal levels of REST function (RESTfl, n=124) and low levels of REST function (RESTless, n=5), and mean RESTfl CELF4 signal intensity was used to normalize CELF4 expression across all tumors. Error bars represent standard error.

FIGS. 32A-32C show that expression of CELF4 or CELF6 is sufficient to permit REST4 splicing. FIG. 32A is a photograph of agarose gel electrophoresis of the results of a qPCR and a graph illustrating REST4 mRNA levels. Stable infection of HEK-293 cells with lentivirus bearing CELF4 (BRUNOL4) or CELF6 (BRUNOL6) coding sequence was sufficient to induce a dramatic increase in REST4 mRNA levels, as measured by qPCR. FIG. 32B is a graph showing that the infection of MCF7 cells with virus bearing either CELF4 (BRUNOL4) or CELF6 was sufficient to induce REST4 expression, as measured by qPCR.

### DETAILED DESCRIPTION OF PREFERRED EMBODIMENTS

The invention is more specifically described below and particularly in the Examples set forth herein, which are intended as illustrative only, as numerous modifications and variations therein will be apparent to those skilled in the art.

As used in the description herein and throughout the claims that follow, the meaning of “a”, “an”, and “the” includes plural reference unless the context clearly dictates otherwise. The terms used in the specification generally have their ordinary meanings in the art, within the context of the invention, and in the specific context where each term is used. Some terms have been more specifically defined below to provide additional guidance to the practitioner regarding the description of the invention.

As described herein, reagents and methods for identifying an aggressive subset of breast cancer tumors is provided, regardless of the status (ER+ or ER−) of estrogen receptor expression in such tumors (a conventional albeit unreliable indicator of tumor aggressiveness). As used herein, the term “aggressive” when used with respect to tumors, particularly breast cancer tumors, will be understood to identify such tumors that are more likely to reoccur and/or metastasize than the majority of breast cancer tumors. As disclosed herein, aggressive breast cancer tumors exhibit altered, typically increased, expression of a subset of cellular genes identified herein as a gene signature. Altered expression of these genes is also shown herein to be associated with production in cells and breast cancer tumor samples of a dysfunctional or non-functional form of a transcription suppressor, termed Neuron Restrictive Silencing Factor (NRSF) and also known as REST (and abbreviated herein as REST/NRSF). The REST/NRSF protein has been identified previously as a putative tumor suppressor and for having a role in cancer progression when reduced expression of REST/NRSF mRNA has been detected in some tumor samples (but specifically not breast cancer). Without wishing to be bound to any mechanistic explanation of the data presented herein, the invention provides reagents and methods for identifying aggressive breast cancer tumors by detecting expression of a gene signature comprising one or a plurality of genes as disclosed herein, or alternatively detecting altered, particularly reduced or aberrant, expression of REST/NRSF in breast cancer tumor samples, or both. In specific embodiments as set forth herein, detection of reduced functional REST/NRSF expression can be achieved by detecting reduced REST protein, increased REST variant protein or decreased native REST mRNA expression accompanied by increased mRNA expression of REST variant species.

As disclosed herein, the gene signatures identified and provided by this invention comprise one or a plurality of cellular genes that have altered, generally increased, expression in tumor samples of aggressive breast cancer tumors. In certain embodiments, increased expression of genes comprising the gene signatures set forth herein are associated with reduced or more particularly aberrant expression of REST/NRSF (termed herein RESTless tumor samples); in particular, RESTless tumors are those that do not show nuclear staining of full-length REST protein as detected inter alia by immunohistochemistry. In some embodiments, REST protein in such tumors was found in the cytoplasm but not the nucleus.

In either embodiment, altered gene expression is relative to less aggressive breast cancer tumor samples, wherein tumor samples expressing the gene signatures of the invention show greater expression of said genes, whereas expression of REST/NRSF is decreased or altered in certain embodiments of said aggressive breast cancer tumor samples. This invention provides such gene signatures and methods of use thereof for identifying aggressive breast cancer tumors, or reduced or dysfunctional REST/NRSF expression, in patient samples and to provide prognoses and diagnoses thereby. It is an advantage of this invention that altered expression of the genes comprising each of the gene signatures provided herein can be readily detected using methods well known to the skilled worker.

In particular embodiments, the invention provides reagents and methods for identifying aggressive breast cancer tumors that are REST/NRSF-deficient. In certain embodiments, the invention provides methods for providing a prognosis of breast cancer patient survival rates for breast cancer patients regardless of the estrogen receptor status (ER+ or ER−) of their tumors. In particular, detection of reduced, altered or aberrant REST/NRSF expression can be used to provide a prognosis of breast cancer patient survival rates for breast cancer patients or to select appropriate cancer therapies.

As disclosed herein, identifying a gene signature of this invention in breast cancer patient tumors can be an independent predictor of poor prognosis in breast cancer. Accordingly, additional embodiments of the invention are directed to using said cancer patient prognosis determined using the gene signatures to select appropriate cancer therapies.

The “gene signatures” are provided in additional aspects of the invention, comprising one or a plurality of genes, the expression of which is altered in aggressive breast cancer tumor samples. As used herein, the term “altered,” “modulated” or “differential” expression includes both increased as well as decreased expression of certain genes, compared to breast tumor samples that are not aggressive. In aggressive breast cancer tumors as disclosed herein, genes comprising gene signatures of the invention exhibit differential expression. In certain embodiments, differential expression comprises increased expression in said certain genes compared to normal breast tissue or REST/NRSF-positive (termed “RESTfl”) tumors. Breast cancer tumor samples expressing gene signatures provided by the invention are identified as described herein. In certain aspects, breast cancers exhibiting more aggressive tumorigenesis and poorer patient survival prognosis are identified by the disclosed methods for detecting such gene signatures. As provided herein, gene signatures comprise one or a plurality of the genes set forth in Tables 1- 4, or 6. In alternative embodiments, aggressive breast cancer tumors are identified and characterized by reduced, altered or aberrant expression of REST/NRSF, and for example the alternative splice variant, REST4.

In a particular embodiment, a gene signature of the invention comprises a single-gene that is LIN28. LIN28 is a tumor promoter gene and a key regulator of miRNA processing. LIN28 is normally expressed during early stages of development, and its upregulation has been associated with multiple aggressive cancers. Two-fold upregulation of LIN28 mRNA promotes metastasis in a mouse model of breast cancer (Dangi-Garimella et. al., 2009, EMBO J 28:347-58). LIN28 promotes tumor progression and metastasis by blocking maturation of the let-7 family of tumor suppressing miRNAs. Multiple members of the let-7 family of miRNAs function as important tumor suppressors in breast tumor initiating cells, and serve to temper expression of multiple breast cancer oncogenes, including c-Myc and Ras, both of which were increased upon REST/NRSF knock-down (Yu, et al., 2007, Cell 131:1109-23; Johnson, et al., 2005, Cell 120:635-47; Sampson, et al., 2007, Cancer Res 67:9762-70; Lee, et al., 2007, Genes Dev 21:1025-30).

In a certain embodiment, a gene signature of the invention comprises one or more of CELF4, CELF5, or CELF6. Without wishing to be bound or limited to any theory or mechanistic explanation, it is shown herein that REST is involved in regulating gene expression of multiple CELF family members, including CELF6, CELF4, and CELF5. All three of these family members are closely related to one another, and are, in many senses, functionally redundant (Barreau et al., 2006, Biochimie, 88:515-525). CELF4-6 all have the ability to enhance inclusion of cTNT exon 5, and CELF4 and CELF6 have also been shown to regulate exon 11 exclusion in the insulin receptor (Barreau et al., 2006, Biochimie, 88:515-525). As set forth herein, overexpression of CELF4 and CELF6 are sufficient to drive REST4 splicing in vitro.

Thus, the term “gene signature” as used herein, and the term “REST/NRSF gene signature,” refers to a collection of cellular genes showing modified, predominantly increased, gene expression in aggressive breast cancer tumor samples. Gene signatures as provided herein can also comprise genes having decreased expression levels, including for example, PTB (polypyrimidine tract binding protein), and thus the skilled worker will appreciate that gene signatures of the invention are characteristic for differential gene expression. In certain embodiments, gene signatures of the invention comprise increased gene expression for genes whose expression is influenced or regulated by REST/NRSF. Gene signatures of the invention can comprise one, about 2, or about 3, or about 4, or about 6, or about 10, or about 20, or about 30, or about 50, or about 75 or about 100 genes; advantageous but non-limiting embodiments of gene signatures as disclosed herein comprise from about 10 to about 20 genes and includes the genes set forth in Tables 1-4, or 6 herein, generally comprising a sufficient number of genes to identify tumors having a poorer patient survival prognosis or showing a shorter patient disease-free survival metric than tumors of the same type and grade, in certain embodiments wherein said aggressive breast cancer tumors have reduced, altered or aberrant expression of REST/NRSF, including splice variants like REST4, as compared to breast cancer tumor samples having functional REST/NRSF. It will be understood that the degree of differential gene expression for members of the REST/NRSF gene signature will vary from specific gene to gene.

The term “differential expression” as used herein refers, but is not limited to, differences in gene expression levels between breast cancer tumor cells or samples characterized as “aggressive” (using tumorigenesis, tumor growth, metastasis, and patient survival as the basis for characterization) compared with other breast cancer tumor samples, or alternatively as breast cancer tumor samples lacking functional REST/NRSF (RESTless) and breast cancer tumor cells or samples expressing the wildtype form and amount of REST/NRSF. Gene expression can be detected by assaying cell or tissue sample as mRNA or protein. In addition, the terms as used herein may refer to gene expression of greater or lesser amounts of mRNA and/or protein in aggressive breast cancer tumor samples compared with normal breast tissue. Alternatively, the term as used herein can refer to gene expression of greater or lesser amounts of mRNA and/or protein in RESTless cell/tumor samples than in normal or REST/NRSF+ cell/tissue samples. The control sample can be from healthy tissue from the same patient or a different patient or a control cell line. “Increased expression” as used herein can also refer to increased expression of a gene product (protein) in a RESTless cell/tumor sample as compared to normal and/or REST/NRSF+ samples.

Detection of a gene signature of the invention can be performed by methods for measuring gene expression levels, including in a non-limiting example conventional microarray techniques described in more detail below. Alternatively, gene expression levels can be detected in certain embodiments by immunoassay or immunohistochemical techniques by detection of the cognate protein products of the members of the gene signature. As used herein with the disclosed methods, gene signatures of this invention identify aggressive subsets of breast cancer tumors (regardless of the status of estrogen receptor expression, the ER+ cohort or ER− cohort) independently of or complementary to other existing predictors of poor prognosis, such as tumor grade, size, patient age and HER2 status. In certain embodiments, the invention provides prognostic indicators of patient disease-free survival times for those patients with tumors otherwise indistinguishable from less aggressive forms of the disease.

The methods provided herein comprise steps for assaying differential gene expression, either of the genes of the gene signatures provided herein or specific genes, including altered genes such as REST/NRSF and miR-124. In these methods, the assays comprise steps of preparing biomolecules, including DNA, RNA, specifically mRNA or cDNA produced therefrom, or RNA or protein products encoded thereby, for said assays. As used herein, said “preparing biomolecules” or said “prepared biomolecules” will be understood to be the products of isolation, extraction or other preparation methods, including but not limited to in situ and immunohistochemistry methods, biochemical purification methods or molecular biological methods such as amplification, cloning, sequencing and converting mRNA to cDNA. Thus said assays will be understood in the art in many embodiments to consume, at least in part, the tumor sample upon which the assays are performed.

In other embodiments of the invention, tumors, particularly breast cancer tumors, exhibiting gene signatures of this invention or reduced or altered expression of functional REST/NRSF as detected using the inventive methods thereby identify patients having reduced disease-free survival times and shorter disease-free survival metrics. In certain embodiments, the invention provides methods for detecting alternative splicing events for REST/NRSF mRNA, illustrated in non-limiting example by REST4, wherein the expressed REST/NRSF protein shows a reduced activity level.

Tissue and tumor samples can be assayed to assess the level of functional REST/NRSF using several methods. These include microarray analysis for detecting the gene signatures disclosed herein. Alternatively, immunohistochemical staining of histological sections from breast cancer tumor samples can be used for staining C-terminal portions of REST/NRSF, alone or together with detection of the REST/NRSF target gene, such as chromogranin-A.

Post-transcriptional regulation of REST/NRSF occurs during neuronal differentiation and oncogenic transformation wherein protein levels thereof can be significantly reduced in the absence of altered mRNA levels (Ballas et al., 2005, Cell 121:645-57; Guardavaccaro et al., 2008, Nature 452:365-69; Westbrook et al., 2008, Nature, 452:370-4). These observations support the findings set forth herein, that REST/NRSF function cannot be directly measured by its mRNA levels in oligonucleotide arrays. However, the development of gene signatures for loss of REST/NRSF in vitro permitted a class of RESTless breast tumors to be identified as set forth herein.

Functional loss of the transcription factor and tumor suppressor REST occurs in multiple aggressive cancers due to the inclusion of a truncating exon, termed the N-exon, in REST mRNA (Coulson et al., 2000, Cancer Res., 60:1840-1844; Wagoner et al., 2010, PLoS Genet 6:e1000979). The N-exon contains a premature stop codon, resulting in the truncation of the REST gene product, thus preventing translation of the second half of the DNA binding or the C-terminal repression domains (Palm et al., 1998, J Neurosci, 18:1280-1296). The resulting protein, termed REST4, lacks the ability to bind DNA or repress transcription, making REST4 a non-functional repressor (Lee et al., 2000, Brain Res Mol Brain Res 80:88-98). In this way, alternative splicing of REST mRNA to include the N-exon depletes cells of full-length REST mRNA, as well as functional REST protein. REST4 was originally identified in the hippocampus following kainic acid-induced seizures and has since been identified in neuroblastoma and pheochromocytoma cell lines, suggesting that it may be a neural splice variant of REST (Palm et al., 1999, Brain Res Mol Brain Res, 72:30-39; Shimojo et al., 1999, Mol Cell Biol, 19:6788-6795; Lee et al., 2000, J Mol Neurosci, 15:205-214). In certain neuroendocrine cancers, loss of REST function by alternative splicing results in exogenous expression of neuronal genes implicated in aggressive cancer (Timmusk et al., 1999, J Biol Chem, 274:1078-1084; Desmet et al., 2006, Cell Mol Life Sci, 63:755-759; Garriga-Canut et al., 2006, Nat Neurosci, 9:1382-1387; Thiele et al., 2009, Clin Cancer Res, 15:5962-5967). In small cell neuroendocrine lung cancer cell lines expressing REST4, introduction of full-length REST induces apoptosis, suggesting that this loss of REST function is key to SCLC cell survival in vitro (Gurrola-Diaz et al., 2003, Oncogene, 22:5636-5645).

It is estimated that 95% of multi-exon genes undergo alternative splicing and at least 50% of these splicing events occur in a cell type-specific manner. The brain is especially enriched in alternative splice variants, driven in part by an array of sequence-specific splicing factors, including neural polypyrimidine tract binding protein (nPTB), neural oncological ventral antigen-1 (NOVA1) and -2 (NOVA2), embryonic lethal abnormal vision (Hu/Elav)-like proteins, CUG binding protein and ETR3-like factor 1 (CELF1), CELF2, and CELF6, many of which are involved in the alternative splicing of neural-specific splice variants (Chen et al., 2009, Nat Rev Mol Cell Biol 10:741-754). Neuronal microRNA miR-124 family members are also known to play a role in neuronal-specific splicing. During neuronal differentiation, miR-124 levels increase following a loss of REST protein (Conaco et al., 2006, Proc Natl Acad Sci USA 103:2422-2427). miR-124 directly binds mRNA encoding the sequence-specific splicing repressor PTB in developing neurons, effectively blocking translation and targeting PTB mRNA for degradation by the RNA-induced silencing complex (Makeyev et al., 2007, Mol Cell, 27:435-448). In non-neural tissues, high levels of PTB protein bind to regulatory elements surrounding exon 10 of nPTB pre-mRNA, resulting in its exclusion from the nPTB transcript and effectively repressing many aspects of neural-specific alternative splicing (Makeyev et al., 2007, Mol Cell, 27:435-448). Inclusion of exon 10 stabilizes the nPTB transcript, resulting in higher levels of the neural-specific splicing protein and neural-specific alternative splicing (Li et al., 2007, Nat Rev Neurosci, 8:819-831).

Alternative splicing is often regulated by a balance of enhancers and inhibitors of exon inclusion (Barreau et al., 2006, Biochimie, 88:515-525; Chen et al., 2009, Nat Rev Mol Cell Biol 10:741-754). A prime example of this is the dynamic antagonism that exists between PTB and the CELF family of sequence-specific splicing regulators (Charlet et al., 2002, Mol Cell, 9:649-658). CELF1 and CELF2 compete with PTB to bind the polypyrimidine tracts within elements known as muscle specific enhancers (MSEs) and, when bound, activate inclusion of exon 5. Relative levels of endogenous PTB and CELF family members determine whether exon 5 is included or excluded by a process of dynamic antagonism.

The CELF proteins are members of the BRUNO-like family of RNA-binding proteins (known as CUG-Binding Protein and embryonic lethal abnormal vision type RNA-binding protein 3 family (CELF) proteins), all of which directly bind pre-mRNA with their RNA recognition motifs (RRM) (Barreau et al., 2006, Biochimie, 88:515-525). CELF family members have highly similar structural organization, with two well-conserved N-terminal RRM domains and a third C-terminal RRM domain separated by a poorly conserved linker region. Each of the six identified CELF proteins is able to activate inclusion of exon 5 in cTNT, with many of the members also able to repress exon inclusion in other genes, such as insulin receptor (Barreau et al., 2006, Biochimie, 88:515-525).

Examples 9 and 10 illustrate that REST regulates numerous aspects of its own alternative splicing by controlling the expression of multiple splicing factors. Loss of REST function results in an increase of miR-124 levels, a decrease of PTB protein levels and an overall increase in REST/NRSF alternative splicing to produce a REST4-encoding transcript. In addition to relieving repression of the N-exon by lowering PTB levels in the cells, loss of REST function also results in the upregulation of CELF4 and CELF6 splicing enhancers. It is shown herein that the exogenous expression of these splicing enhancers is sufficient to increase REST4 splicing. PTB and CELF4/CELF6 dynamically antagonize the inclusion of the REST N-exon in breast tumor cell lines, the balance of which is determined by REST itself.

In other embodiments, the invention provides methods for detecting functional REST/NRSF expression levels, wherein breast cancer tumors having reduced functional REST/NRSF expression levels identify patients having reduced disease-free survival times and shorter disease-free survival metrics. In the application and practice of these inventive methods, any method known in the art for detecting aberrant or dysfunctional REST/NRSF mRNA species can be used, including allele-specific polymerase chain reaction, nucleotide sequence analysis, specific hybridization assays, or combinations of said methods. In alternative embodiments, REST/NRSF protein is assayed, using methods including but not limited to immunoassay and immunohistochemical (IHC) methods well known in the art. In certain embodiments, these methods are practiced by identifying expression of REST4 in breast cancer tumor samples, wherein said breast cancer tumor samples identify patients having reduced disease-free survival times and shorter disease-free survival metrics. In alternative embodiments, IHC methods are used to detect breast cancer tumors expressing altered REST/NRSF, wherein particular embodiments are directed towards differential detection of amino-terminal and particularly carboxyl-terminal portions of REST/NRSF. In particular examples, methods for immunohistochemical detection of ER− breast cancer tumors deficient for REST/NRSF expression are provided.

As used herein, a “patient” or “subject” to be treated by the disclosed methods can mean either a human or non-human animal but in certain particular embodiments is a human.

The term “patient sample” as used herein refers to a cell or tissue sample obtained from a patient (such as a biopsy) or cells collected from in vitro cultured samples; the term can also encompass experimentally derived cell samples.

As used herein, the term “tumor sample” refers to a diseased or cancerous tissue sample including specifically cell culture samples, experimentally derived samples, biopsy samples and other samples obtained from a subject and comprising a malignant or putatively malignant tumor. In particular, the term refers to a breast cancer sample. The term “tumor” refers to a tissue sample or cells that exhibit a cancerous morphology, express cancer markers, or appear abnormal, or that have been removed from a patient having a clinical diagnosis of cancer. A tumor or “tumorigenic tissue” is not limited to any specific stage of cancer or cancer type, and include in non-limiting examples dysplasia, anaplasia and precancerous lesions. As used herein, the term “disease” or “diseased” refers to any abnormal proliferative pathology, including but not limited to cancer. As used herein, the term “aberrant” refers to abnormal or altered. The term “aggressive” as used herein to describe but is not limited to tumors associated with reduced patient prognosis and/or survival rate, tumors that increase in size and/or metastasize at a faster rate, or tumors of a more severe grade (i.e., higher grades) that other tumor of the same origin. In particular, the invention provides reagents and methods for identifying breast cancer tumor patients having reduced patient survival times, more aggressive tumors and poorer prognosis.

As used herein, the term “biomolecule(s)” refers to DNA, RNA or protein isolated from a sample (e.g., a tumor sample). Said biomolecules include but are not limited to mRNA, cDNA, miRNA, DNA, nucleic acid fragments, peptides, peptide fragments, partial protein domains, or full-length proteins in either (native or denatured state).

The practice of the inventive methods can involve established molecular biology procedures, including for example, nucleotide sequence amplification, such as polymerase chain reaction (PCR) and modifications thereof (including for example reverse transcription (RT-PCR), and stem-loop PCR, qPCR, as well as reverse transcription and in vitro transcription. Generally these methods utilize one or a pair of oligonucleotide primers having sequence complimentary to sequences 5′ and 3′ to the sequence of interest. In their use these primers are hybridized to a nucleotide sequence and extended during the practice of PCR amplification using DNA polymerase (preferably using a thermal-stable polymerase such as Taq polymerase). RT-PCR may be performed on mRNA with a specific 5′ primer or random primers and appropriate reverse transcription enzymes such as avian (AMV-RT) or murine (MMLV-RT) reverse transcriptase enzymes to convert RNA to cDNA. Specific, non-limiting examples of such methods for assessing gene expression levels useful in the practice of the inventive methods use reverse transcriptase real time polymerase chain reaction (RT-RTPCR). Use of PCR-based methods including RT-RTPCR advantageously permits rapid, inexpensive and accurate measurement of tens to hundreds of genes simultaneously, and can be used to track gene signatures in breast cancer. As will be understood in the art, reagents for performing many of these analytic methods are commercially available.

As used herein, the terms “microarray,” “bioarray,” “biochip” and “biochip array” refer to an ordered spatial arrangement of immobilized biomolecular probes arrayed on a solid supporting substrate. Advantageously, the biomolecular probes are immobilized on the solid supporting substrate.

Gene arrays or microarrays as known in the art are useful in the practice of the methods of this invention. See, for example, DNA MICROARRAYS: A PRACTICAL APPROACH, Schena, ed., Oxford University Press: Oxford, UK, 1999. As used in the methods of the invention, gene arrays or microarrays comprise a solid substrate, preferably within a square of less than about 22 mm by 22 mm on which a plurality of positionally distinguishable polynucleotides are attached at a diameter of about 100-200 microns. These probe sets can be arrayed onto areas of up to 1 to 2 cm², providing for a potential probe count of >30,000 per chip. The solid substrate of the gene arrays can be made out of silicon, glass, plastic or any suitable material. The form of the solid substrate may also vary and may be in the form of beads, fibers or planar surfaces. The sequences of the polynucleotides comprising the array are preferably specific for human mRNA or miRNA. The polynucleotides are attached to the solid substrate using methods known in the art (Schena, Id.) at a density at which hybridization of particular polynucleotides in the array can be positionally distinguished. Preferably, the density of polynucleotides on the substrate is at least 100 different polynucleotides per cm², more preferably at least 300 polynucleotides per cm². In addition, each of the attached polynucleotides comprises at least about 25 to about 50 nucleotides and has a predetermined nucleotide sequence. Target RNA or cDNA preparations are used from tumor samples that are complementary to at least one of the polynucleotide sequences on the array and specifically bind to at least one known position on the solid substrate. Such microarrays and uses thereof are well known in the art (see, for example, Lockhart et al., 2000, Nature 405: 827-36; Schena et al., 1998, Trends Biotechnol. 16: 301-6; Schadt et al., 2000, J. Cell Biochem. 80: 192-202; Li et al., 2001, Bioinformatics 17: 1067-1076; Wu et al., 2001, Appl. Environ. Microbiol. 67: 5780-90; and Kaderali et al., 2002, Bioinformatics 18: 1340-9).

Two principal array platforms are currently in widespread use, but differ in how the oligonucleotide probes are placed onto the hybridization surface (Lockhart et al., 2000, Id. and Gerhold et al., 1999, Trends Biochem. Sci. 24: 168-73). Schena and Brown pioneered techniques for robotically depositing presynthesized oligonucleotides (typically, PCR-amplified inserts from cDNA clones) onto coated surfaces (Schena et al., 1995, Science 270: 467-70 and Okamoto et al., 2000, Nat. Biotechnol. 18: 438-41). Fodor et al. (1991, Science 251: 767-73) and Lipshutz et al. (1999, Nat. Genet. 21:20-4), on the other hand, utilized photolithographic masking techniques (similar to those used to manufacture silicon chips) to construct polynucleotides one base at a time on preferentially unmasked surfaces containing an oligonucleotide targeted for chain elongation. These two methods generate reproducible probe sets amenable for gene expression profiling and can be used to determine the gene expression profiles of tumor samples when used in accordance with the methods of this invention.

Biochips, as used in the art, encompass substrates containing arrays or microarrays, preferably ordered arrays and most preferably ordered, addressable arrays, of biological molecules that comprise one member of a biological binding pair. Typically, such arrays are oligonucleotide arrays comprising a nucleotide sequence that is complementary to at least one sequence that may be or is expected to be present in a biological sample. As provided herein, the invention comprises useful microarrays for detecting differential expression in tumor samples, prepared as set forth herein or provided by commercial sources, such as Affymetrix, Inc. (Santa Clara, Calif.), Incyte Inc. (Palo Alto, Calif.) and Research Genetics (Huntsville, Ala.).

In certain embodiments, said biochip arrays are used to detect differential expression of target mRNA or miRNA species by hybridizing amplification products from experimental and control tissue samples to said array, and detecting hybridization at specific positions on the array having known complementary sequences to specific mRNA or miRNA target(s).

In certain other embodiments of the diagnostic methods of this invention, expression of the protein product(s) of mRNA targets are detected. In some embodiments, protein products are detected using immunological reagents, examples of which include antibodies, most preferably monoclonal antibodies that recognize said differentially-expressed proteins.

For the purposes of this invention, the term “immunological reagents” is intended to encompass antisera and antibodies, particularly monoclonal antibodies, as well as fragments thereof (including F(ab), F(ab)₂, F(ab)′ and Fᵥ fragments). Also included in the definition of immunological reagent are chimeric antibodies, humanized antibodies, and recombinantly-produced antibodies and fragments thereof. Immunological methods used in conjunction with the reagents of the invention include direct and indirect (for example, sandwich-type) labeling techniques, immunoaffinity columns, immunomagnetic beads, fluorescence activated cell sorting (FACS), enzyme-linked immunosorbent assays (ELISA), radioimmuno assay (RIA), as well as peroxidase labeled secondary antibodies that detect the primary antibody.

The immunological reagents of the invention are preferably detectably labeled, most preferably using fluorescent labels that have excitation and emission wavelengths adapted for detection using commercially-available instruments such as and most preferably fluorescence activated cell sorters. Examples of fluorescent labels useful in the practice of the invention include phycoerythrin (PE), fluorescein isothiocyanate (FITC), rhodamine (RH), Texas Red (TX), Cy3, Hoechst 33258, and 4′,6-diamidino-2-phenylindole (DAPI), as well as those labels specifically described in the Examples section. Such labels can be conjugated to immunological reagents, such as antibodies and most preferably monoclonal antibodies using standard techniques (Maino et al., 1995, Cytometry 20: 127-133).

The invention also provides kits for performing the methods disclosed herein. In certain embodiments, the kits of this invention comprise an antibody specific for the C-terminus of REST/NRSF protein, wherein in particular embodiments said antibody can be a monoclonal antibody, an antisera, or a plurality of antibodies recognizing aberrant or wildtype species of REST/NRSF protein. Optionally included in specific embodiments of the kits of the invention can be instructions for use, as well as secondary antibodies useful inter alia in sandwich assays understood by those in the art. Distinguishingly labeled embodiments of the antibody components of said kits, as well as reagents and methods for labeling said antibodies, are also advantageously-provided components of the kits of the invention.

In other embodiments, kits of the invention comprise one or plurality of oligonucleotide primers that each specifically hybridize to one or a plurality of the genes identified in Table 1, 2, 3, 4, or 6. In certain embodiments, said oligonucleotides are provided on a solid support, including without limitation chips, microarrays, beads and the like. Optionally included in specific embodiments of the kits of the invention can be instructions for use. Distinguishingly labeled embodiments of the oligonucleotide components of said kits, as well as reagents and methods for labeling said oligonucleotides, are also advantageously-provided components of the kits of the invention.

In further embodiments, kits of the invention comprise one or plurality of immunological reagents, particularly antibodies that each specifically bind to a protein produced by increased expression of one or a plurality of the genes identified in Table 1, 2, 3, 4 or 6. In certain embodiments, said immunological reagents, particularly antibodies are provided on a solid support, including without limitation chips, microarrays, beads and the like. Optionally included in specific embodiments of the kits of the invention can be instructions for use, as well as secondary antibodies useful inter alia in sandwich assays understood by those in the art. Distinguishingly labeled embodiments of the immunological reagent components of said kits, particularly antibodies, as well as reagents and methods for labeling said antibodies, are also advantageously-provided components of the kits of the invention.

The kits of the invention are useful for diagnosing or prognosing reduced disease-free survival time in a human with cancer, particularly breast cancer and in specific embodiments aggressive breast cancer in human cancer patients

Embodiments of the methods of this invention comprising the above-mentioned features are intended to fall within the scope of this invention.

### EXAMPLES

The Examples that follow are illustrative of specific embodiments of the invention, and various uses thereof They set forth for explanatory purposes only, and are not to be taken as limiting the invention.

### Example 1

### Identification of Gene Signatures in Breast Cancer Cells

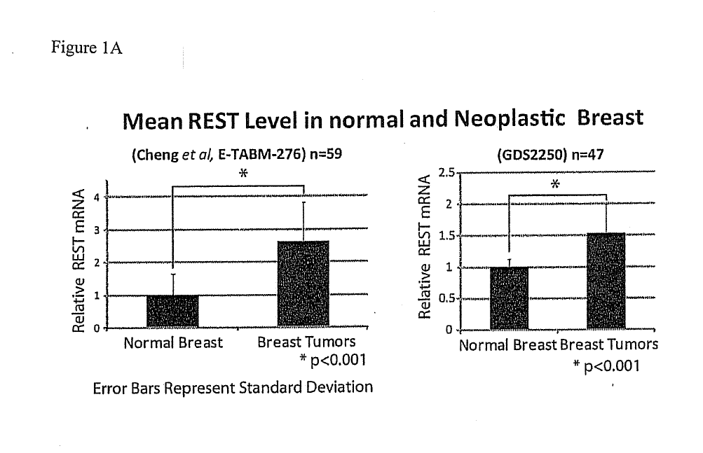

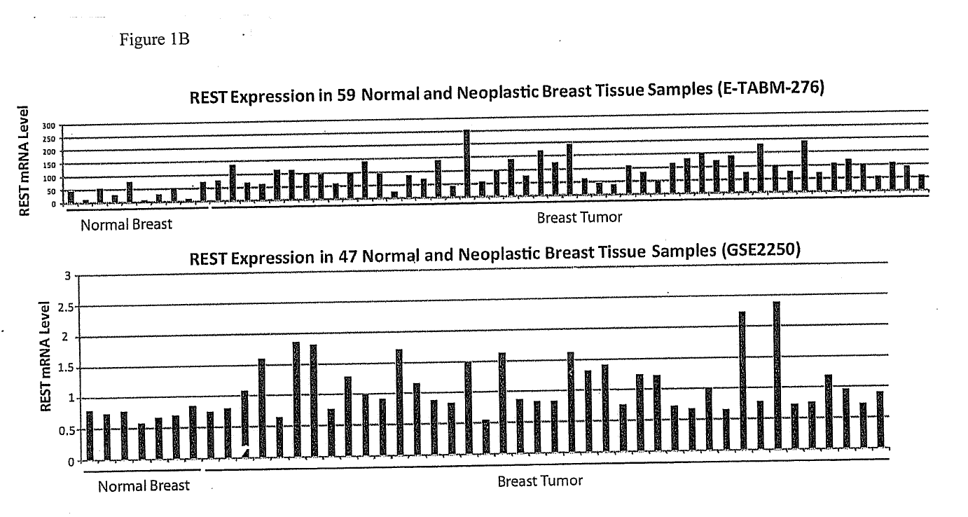

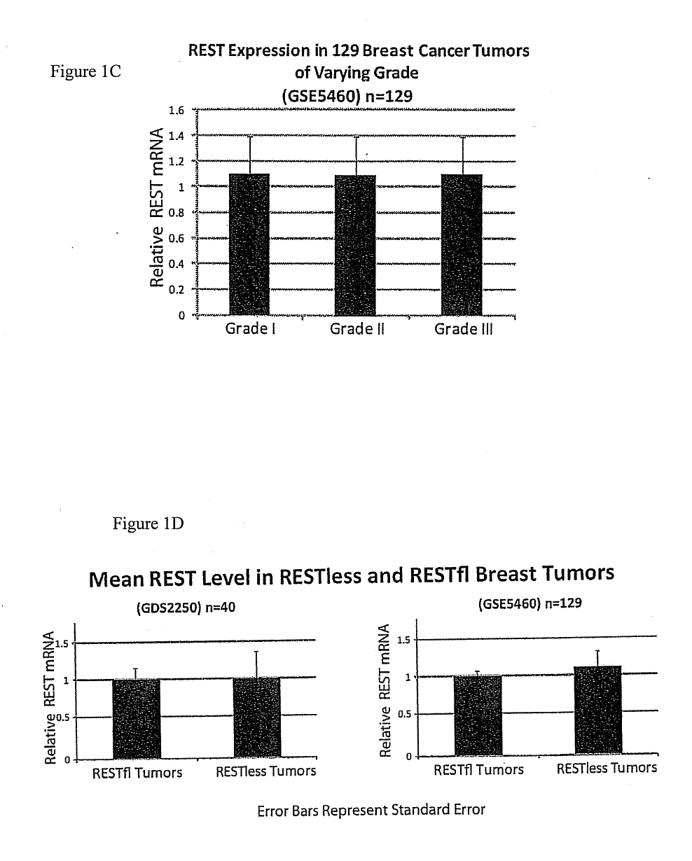

Assays of breast cancer tumor samples for REST/NRSF mRNA levels did not show a decrease in REST/NRSF mRNA, a result that was not expected in view of results of chromosomal loss-of-heterozygosity studies on colon cancer (Westbrook et al., 2005, Cell 121:837-848). Specifically, DNA microarray assays of normal and neoplastic breast tissue were performed as set forth herein, and indicated that REST/NRSF mRNA levels were similar across tumors and normal mammary tissue (as shown FIGS. 1A through 1D). In view of this result, which was inconsistent with expectations from other tumors shown in the art, REST/NRSF function was specifically inhibited experimentally in three cell lines, to determine whether REST/NRSF played any role in the etiology of breast cancer. For these experiments, two human mammary (MCF10a and T47D) and one human embryonic kidney (HEK-293) cell line were experimentally manipulated so that each of these cell lines had REST/NRSF expression reduced (so-called gene “knocked-down” experiments).

Stable REST/NRSF-knockdown cell lines were generated using a lentivirus-based system, commercially available from Thermo Fisher Dharmacon (Lafayette, Colo.) called SMART Vector shRNA lentiviral particles. Lentiviral particles comprising a nucleic acid encoding a shRNA were used to infect HEK-293 (human embryonic kidney cells), T47D (a breast cancer-derived cell line) and MCF10a (mammary epithelial) cells with either a non-targeting shRNA or an shRNA specific to REST/NRSF (Catalog #S-00500-01 and SH-042194-01-25, respectively) according to the manufacturer's instructions. Briefly, 2×10⁵ cells of each cell type were plated in a 96 well tray overnight, and infected the following morning with 1×10⁶ viral particles in normal medium containing polybrene. Medium was changed after 8 hours of infection. Cells that stably integrated the shRNA into their genome were selected 48 hours after infection using puromycin, and verified for REST/NRSF knockdown via Western Blot analysis with anti-REST specific antibodies (anti-REST antibody was obtained from Millipore, Catalog #07-579, Billerica, Mass.).

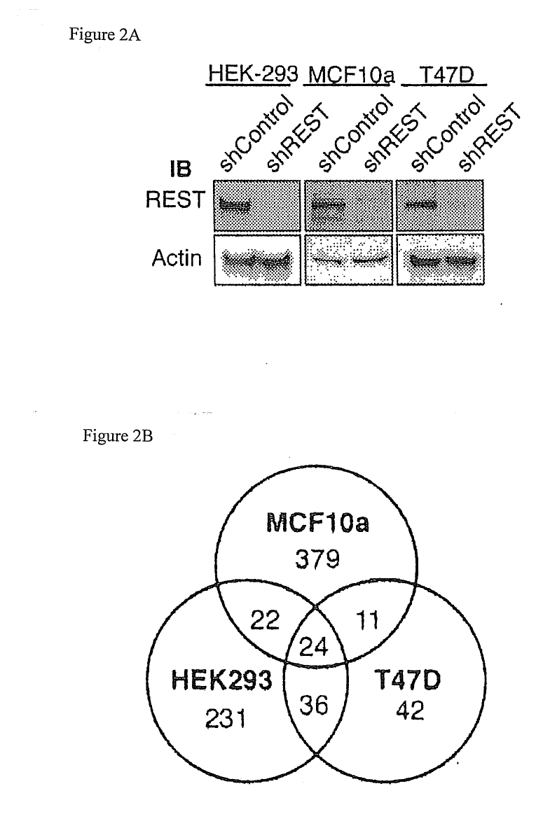

The results of these experiments are shown in FIG. 2A. Breast cancer cells having REST/NRSF knocked down using shRNA grew almost twice as fast as control cells. The increased growth rate observed in breast cancer cells following REST/NRSF knockdown suggested that loss of REST/NRSF produced more aggressive tumor growth. In addition to increased growth rate, reduced expression of REST/NRSF in these cells resulted in increased expression of several genes in all three cell types. These genes were identified by microarray analysis comparing gene expression levels in REST/NRSF knockdown cells with controls expressing the native amounts of REST/NRSF. In these experiments, RNA was extracted from 10⁷ cells from each group of knockdown and control cell lines in duplicate using Trizol Reagent (Invitrogen, Carlsbad, Calif.) according to the manufacturer's instructions. Six DNA microarrays were used (Roche—Nimblegen HG18 60 mer Gene Expression Arrays, Catalog #A4542-00-01) in these experiments, wherein each of the six arrays were dual hybridized with a control and a knockdown aRNA (i.e., amplified RNA) sample from each cell type, in duplicate. Cy3 and Cy5 fluorophores were alternatively used to label the aRNA from control and knockdown cell lines (dye swap) to control any fluorophore-induced effects.

REST/NRSF target genes that were consistently and robustly elevated in the absence of functional REST/NRSF were identified from these experiments. In determining which genes satisfied the criteria for consistency and robustness, microarray data analyses were performed using GeneSifter microarray analysis software to determine which genes were the most consistently and robustly upregulated upon REST/NRSF knockdown between cell lines. In analyzing these results, genes were scored as being “REST/NRSF target genes” if a two-fold upregulation for each gene in response to REST/NRSF knockdown was detected in at least two of the tested cell lines. This analysis yielded 93 genes, which are listed in Tables 1 and 2.

Twenty-four genes highly and consistently upregulated two-fold or more upon REST/NRSF knockdown across three cell lines (FIG. 2B) are set out in Table 1.

TABLE 1
REST/NRSF target genes upregulated across three cell lines MCF10a, HEK,
T47D.
(This twenty-four gene subset was termed the “24-gene signature” and is a non-limiting
example of one embodiment of the invention.)

| Gene | | Transcript Accession | |
|---|---|---|---|
| Abbrev. | Gene Name | No.* | SEQ ID NO: |
| AP3B2 | Adaptor-related protein | ENST00000261722 | SEQ ID NO: 1 |
| | complex 3, beta 2 subunit | | |
| BSN | Bassoon (presynaptic | NM_003458 | SEQ ID NO: 2 |
| | cytomatrix protein) | | |
| CHGB | Chromogranin B (secretogranin | ENST00000203005 | SEQ ID NO: 3 |
| | 1) | | |
| CPLX1 | Complexin 1 | ENST00000445104 | SEQ ID NO: 4 |
| CPLX2 | Complexin 2 | NM_006650 | SEQ ID NO: 5 |
| DISP2 | Dispatched homolog 2 | ENST00000254616 | SEQ ID NO: 6 |
| | (Drosophila) | | |
| GOLGA7B | Golgi Autoantigen 7B | ENST00000370602 | SEQ ID NO: 7 |
| HBA1 | Hemoglobin alpha 1 | ENST00000320868 | SEQ ID NO: 8 |
| HBA2 | Hemoglobin alpha 2 | ENST00000251595 | SEQ ID NO: 9 |
| KCNB1 | Potassium voltage-gated | ENST00000371741 | SEQ ID NO: 10 |
| | channel, Shab-related | | |
| | subfamily, member 1 | | |
| MAPK8IP2 | Mitogen-activated protein | ENST00000329492 | SEQ ID NO: 11 |
| | kinase 8 interacting protein 2 | | |
| MMP24 | Matrix metallopeptidase 24 | ENST00000246186 | SEQ ID NO: 12 |
| | (membrane-inserted) | | |
| PGBD5 | PiggyBac transposable element | ENST00000321327 | SEQ ID NO: 13 |
| | derived 5 | | |
| RLTPR | RGD motif, leucine rich repeats, | ENST00000334583 | SEQ ID NO: 14 |
| | tropomodulin domain and | | |
| | proline-rich containing | | |
| RTN2 | Reticulon 2 | ENST00000245923 | SEQ ID NO: 15 |
| RUNDC3A | RUN domain containing 3A | NM_001144825 | SEQ ID NO: 16 |
| SCAMP5 | Secretory carrier membrane | NM_138967 | SEQ ID NO: 17 |
| | protein 5 | | |
| SCGB1D2 | Secretoglobin, family 1D, | ENST00000244926 | SEQ ID NO: 18 |
| | member 2 (Lipophilin B) | | |
| SNAP25 | Synaptosomal-associated | NM_003081 | SEQ ID NO: 19 |
| | protein, 25 kDa | | |
| STMN3 | Stathmin-like 3 | ENST00000358145 | SEQ ID NO: 20 |
| SYP | Synaptophysin | ENST00000263233 | SEQ ID NO: 21 |
| TMEM145 | Transmembrane protein 145 | ENST00000406159 | SEQ ID NO: 22 |
| TMEM198 | Transmembrane protein 198 | ENST00000373883 | SEQ ID NO: 23 |
| VGF | VGF nerve growth factor | ENST00000249330 | SEQ ID NO: 24 |
| | inducible | | |

*Transcript accession numbers beginning “ENST” are from the Ensembl Project database; all other accession numbers are from GenBank

TABLE 2
Genes that are highly and consistently upregulated in 2 cell lines.

| | | | |
|---|---|---|---|
| | | Transcript Accession | |
| Gene Abbrev. | Gene Name | No.* | SEQ ID NO: |

HEK and T47D cell lines:

| | | | |
|---|---|---|---|
| ACTL6B | | ENST00000160382 | SEQ ID NO: 25 |
| BEX1 | brain expressed, X- | ENST00000255533 | SEQ ID NO: 26 |
| | linked 1 | | |
| BRUNOL6 | | ENST00000287202 | SEQ ID NO: 27 |
| C3orf14 | | ENST00000494481 | SEQ ID NO: 28 |
| CAMK2N2 | Homo sapiens | NM_033259 | SEQ ID NO: 29 |
| | calcium/calmodulin- | | |
| | dependent protein | | |
| | kinase II inhibitor 2 | | |
| CECR6 | Cat eye syndrome | ENST00000331437 | SEQ ID NO: 30 |
| | critical region protein 6 | | |
| DIRAS1 | DIRAS family, GTP- | ENST00000321327 | SEQ ID NO: 31 |
| | binding Ras-like 1 | | |
| FGF12 | Fibroblast growth | ENST00000454309 | SEQ ID NO: 32 |
| | factor 12 | | |
| FLJ40125 | Probable protein | ENST00000451287 | SEQ ID NO: 33 |
| | phosphatase 1B-like | | |
| GABRD | Gamma- | ENST00000344115 | SEQ ID NO: 34 |
| | aminobutyric-acid | | |
| | receptor delta subunit | | |
| | precursor (GABA(A) | | |
| | receptor) | | |
| GNAO1 | Guanine nucleotide | ENST00000262493 | SEQ ID NO: 35 |
| | binding protein (G | | |
| | protein), alpha | | |
| | activating activity | | |
| | polypeptide O | | |
| GNG4 | Guanine nucleotide- | ENST00000302505 | SEQ ID NO: 36 |
| | binding protein | | |
| | G(I)/G(S)/G(O) | | |
| | gamma-4 subunit | | |
| HRH3 | Histamine receptor | ENST00000370797 | SEQ ID NO: 37 |
| | H3 | | |
| INSM2 | Insulinoma- | ENST00000307169 | SEQ ID NO: 38 |
| | associated 2 | | |
| KCNK3 | Potassium channel, | ENST00000302909 | SEQ ID NO: 39 |
| | subfamily K, member 3 | | |
| LIN28 | Lin-28 homolog A | ENST00000326279 | SEQ ID NO: 40 |
| | (Zinc finger CCHC | | |
| | domain-containing | | |
| | protein 1) | | |
| NFASC | Homo sapiens | NM_015090 | SEQ ID NO: 41 |
| | neurofascin homolog | | |
| | (chicken) | | |
| OLFM1 | Olfactomedin 1 | ENST00000371793 | SEQ ID NO: 42 |
| PSD | PH and SEC7 | ENST00000020673 | SEQ ID NO: 43 |
| | domain-containing | | |
| | protein 1 | | |
| PTPRH | Protein tyrosine | ENST00000376350 | SEQ ID NO: 44 |
| | phosphatase, receptor | | |
| | type, H | | |
| RAB3C | RAB3C, member Ras | ENST00000381158 | SEQ ID NO: 45 |
| | oncogene family | | |
| RELL2 | Homo sapiens RELT- | NM_173828 | SEQ ID NO: 46 |
| | like 2, transcript | | |
| | variant 1 | | |
| RIPPLY2 | Protein ripply2 | ENST00000369687 | SEQ ID NO: 47 |
| RTBDN | Retbindin | ENST00000322912 | SEQ ID NO: 48 |
| SBK1 | Serine/threonine- | ENST00000341901 | SEQ ID NO: 49 |
| | protein kinase SBK1 | | |
| SCN8A | Sodium channel | ENST00000354534 | SEQ ID NO: 50 |
| | protein type 8 subunit | | |
| | alpha (Sodium | | |
| | channel protein type | | |
| | VIII subunit | | |
| | alpha) (Voltage-gated | | |
| | sodium channel | | |
| | subunit alpha | | |
| | Nav1.6) | | |
| SLC5A5 | Solute carrier family | ENST00000222248 | SEQ ID NO: 51 |
| | 5 (sodium iodide | | |
| | symporter), member 5 | | |
| SLC6A17 | Hypothetical protein | ENST00000450985 | SEQ ID NO: 52 |
| | LOC284462 | | |
| SLC8A2 | Solute carrier family | ENST00000236877 | SEQ ID NO: 53 |
| | 8 (sodium-calcium | | |
| | exchanger), member 2 | | |
| SMPD3 | sphingomyelin | ENST00000219334 | SEQ ID NO: 54 |
| | phosphodiesterase 3, | | |
| | neutral membrane | | |
| SPTBN4 | Spectrin, beta, non- | ENST00000338932 | SEQ ID NO: 55 |
| | erythrocytic 4 | | |
| STX1A | Syntaxin-1A | ENST00000222812 | SEQ ID NO: 56 |
| | (Neuron-specific | | |
| | antigen HPC-1) | | |
| SYN1 | Synapsin-1 (Synapsin | ENST00000263237 | SEQ ID NO: 57 |
| | I) (Brain protein 4.1) | | |
| TCL6 | T-cell | ENST00000341772 | SEQ ID NO: 58 |
| | leukemia/lymphoma 6 | | |
| TMEM151A | Transmembrane | ENST00000327259 | SEQ ID NO: 59 |
| | protein 151A | | |
| TMEM180 | Chromosome 10 open | ENST00000238936 | SEQ ID NO: 60 |
| | reading frame 77 | | |

HEK and MCF10a cell lines:

| | | | |
|---|---|---|---|
| ASPHD1 | Aspartate beta- | ENST00000308748 | SEQ ID NO: 61 |
| | hydroxylase domain- | | |
| | containing protein 1 | | |
| CABP1 | Calcium binding | ENST00000316803 | SEQ ID NO: 62 |
| | protein 1 (calbrain) | | |
| CD24 | CD24 antigen (small | ENST00000382840 | SEQ ID NO: 63 |
| | cell lung carcinoma | | |
| | cluster 4 antigen) | | |
| CDK5R2 | Cyclin-dependent | ENST00000308748 | SEQ ID NO: 64 |
| | kinase 5, regulatory | | |
| | subunit 2 (p39) | | |
| CPNE4 | Copine IV (Copine 8) | ENST00000357965 | SEQ ID NO: 65 |
| CPNE4 | Copine IV (Copine 8) | ENST00000354260\hat{ } | SEQ ID NO: 66 |
| | \hat{ } the most representative | | |
| | transcript of the three | | |
| CPNE4 | Copine IV (Copine 8) | ENST00000356700 | SEQ ID NO: 67 |
| CRABP2 | Cellular retinoic acid | ENST00000368222 | SEQ ID NO: 68 |
| | binding protein 2 | | |
| DNER | Delta and Notch-like | ENST00000341772 | SEQ ID NO: 69 |
| | epidermal growth | | |
| | factor-related | | |
| | receptor Precursor | | |
| DRD2 | Dopamine receptor | ENST00000355319 | SEQ ID NO: 70 |
| | D2 | | |
| FAM155B | Transmembrane | ENST00000252338 | SEQ ID NO: 71 |
| | protein FAM155B | | |
| | (Transmembrane | | |
| | protein 28) (Protein | | |
| | TED) | | |
| FSTL5 | Follistatin-like 5 | ENST00000306100 | SEQ ID NO: 72 |
| MAPK11 | Mitogen-activated | ENST00000330651 | SEQ ID NO: 73 |
| | protein kinase 11 | | |
| MARCH4 | Membrane-associated | ENST00000273067 | SEQ ID NO: 74 |
| | ring finger (C3HC4) 4 | | |
| MEIS3 | MEIS1, myeloid | ENST00000331559 | SEQ ID NO: 75 |
| | ecotropic viral | | |
| | integration site 1 | | |
| | homolog 3 (mouse) | | |
| OGDHL | oxoglutarate | ENST00000355036 | SEQ ID NO: 76 |
| | dehydrogenase-like | | |
| PCBP3 | Poly(RC) binding | ENST00000400310 | SEQ ID NO: 77 |
| | protein 3 | | |
| PHF21B | Homo sapiens PHD | NM_001135862 | SEQ ID NO: 78 |
| | finger protein 21B, | | |
| | transcript variant 2 | | |
| RAB11FIP4 | RAB11 family | ENST00000325874 | SEQ ID NO: 79 |
| | interacting protein 4 | | |
| | (class ii) | | |
| RTN2 | Reticulon 2 | ENST00000245923 | SEQ ID NO: 15 |
| SCN3B | Sodium channel, | ENST00000406159 | SEQ ID NO: 80 |
| | voltage-gated, type | | |
| | III, beta | | |
| SEZ6L2 | seizure related 6 | ENST00000350527 | SEQ ID NO: 81 |
| | homolog (mouse)- | | |
| | like 2 | | |
| SYT14 | Synaptotagmin XIV | ENST00000422431 | SEQ ID NO: 82 |

MCF10a and T47D cell lines:

| | | | |
|---|---|---|---|
| ATL1 | Atlastin-1 (Guanine | ENST00000358385 | SEQ ID NO: 83 |
| | nucleotide-binding | | |
| | protein 3) (GTP- | | |
| | binding protein 3) | | |
| | (GBP-3) (Brain- | | |
| | specific GTP-binding | | |
| | protein) | | |
| CKMT1B | Homo sapiens | NM_020990 | SEQ ID NO: 84 |
| | creatine kinase, | | |
| | mitochondrial 1B, | | |
| | nuclear gene | | |
| | encoding | | |
| | mitochondrial protein | | |
| ENOX1 | Ecto-NOX disulfide- | ENST00000261488 | SEQ ID NO: 85 |
| | thiol exchanger 1 | | |
| | (Constitutive Ecto- | | |
| | NOX) (cNOX) (Candidate | | |
| | growth-related | | |
| | and time keeping | | |
| | constitutive | | |
| | hydroquinone | | |
| | [NADH] | | |
| | oxidase) (cCNOX) (Cell | | |
| | proliferation- | | |
| | inducing gene 38 | | |
| | protein) | | |
| FBXL15 | F-box and leucine- | ENST00000224862 | SEQ ID NO: 86 |
| | rich repeat protein 15 | | |
| GDAP1 | Ganglioside-induced | ENST00000220822 | SEQ ID NO: 87 |
| | differentiation- | | |
| | associated protein 1 | | |
| | (GDAP1) | | |
| LOC283174 | Uncharacterized | ENST00000421172 | SEQ ID NO: 88 |
| | protein LOC283174 | | |
| MAPK8IP1 | Mitogen-activated | ENST00000241014 | SEQ ID NO: 89 |
| | protein kinase 8 | | |
| | interacting protein 1 | | |
| PCDHA6 | Homo sapiens | NM_018909 | SEQ ID NO: 90 |
| | protocadherin alpha | | |
| | 6, transcript variant 1 | | |
| RIMKLA | Ribosomal protein S6 | ENST00000372570 | SEQ ID NO: 91 |
| | modification-like | | |
| | protein A | | |
| SH3GLB1 | SH3-domain GRB2- | ENST00000212369 | SEQ ID NO: 92 |
| | like endophilin B1 | | |
| TRIM9 | Tripartite motif | ENST00000298355 | SEQ ID NO: 93 |
| | protein 9 (RING | | |
| | finger protein 91) | | |

*Transcript accession numbers beginning “ENST” are from the Ensembl Project database; all other accession numbers are from GenBank.

TABLE 3
Example of Single-Gene Gene Signature

| | Gene | Transcript | |
|---|---|---|---|
| Gene Name | Abbreviation | Accession No. | SEQ ID NO |
| LIN28 | Lin-28 | ENST00000326279 | SEQ ID NO: 40 |
| | homolog A | | |
| | (Zinc finger | | |
| | CCHC | | |
| | domain- | | |
| | containing | | |
| | protein 1) | | |

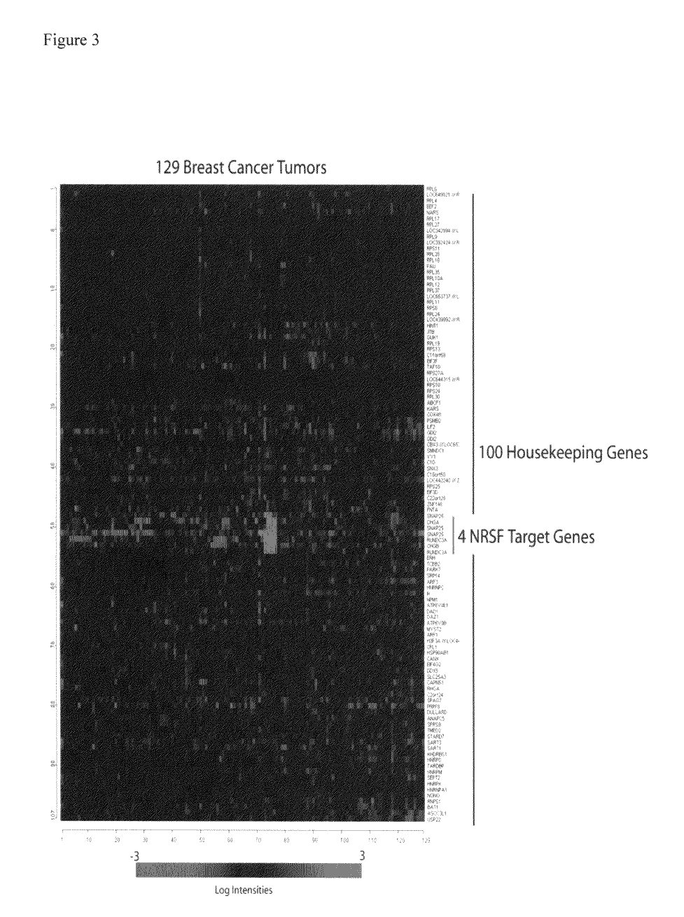

In addition to the subsets of genes identified from the cell line studies described above, analyses of differential gene expression of a collection of breast cancer tumor samples was also performed. The GSE5460 breast cancer tumor set was divided into two phenotypes: those established as deficient for REST function (RESTless) and those with functional REST (RESTfl). The “24 gene signature” was used to screen the tumors and increased expression of the signature genes was observed for those tumors with the RESTless phenotype; these results are set forth in FIG. 3. These microarray results for gene expression from breast cancer tumor samples showed increased mRNA expression levels (shown in red) of particular cellular genes (the “24 gene signature,” identified on the righthand side of the array) in 129 breast cancer tumors (identified across the top border of the array). Thus, elevated expression of a “gene signature” was correlated with REST-deficient tumors (see Table 4).

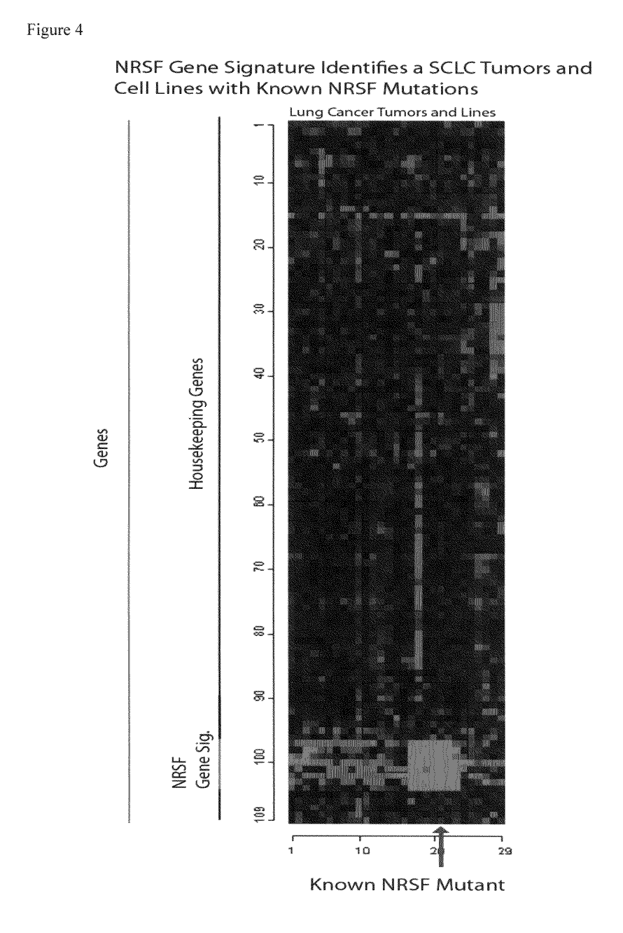

These results were compared with alterations of gene expression found in neuroendocrine tumors found in certain small cell lung cancers, which have been shown to express aberrantly spliced REST/NRSF (Coulson, 2000, Cancer Res. 60:1840-4; Gurrola-Diaz, 2003, Oncogene 22: 5636-5645). These results are shown in FIG. 4, wherein gene expression for several of the genes comprising the 24-gene signature detected in breast cancer cells with reduced REST/NRSF expression are likewise overexpressed in these cells.

The significance of the gene expression profiles detected as set forth in this example was determined by analyses of tumor progression, disease outcomes and survival from clinical tumor samples, as set forth below.

### Example 2

### Tumors Exhibiting REST/NRSF Gene Signatures have Reduced Patient Survival Rates

Breast cancer microarrays were queried for those cancers exhibiting a REST/NRSF gene signature as disclosed herein. Microarray data from 211 estrogen receptor positive (ER+) breast cancer patients were screened for the REST/NRSF gene signature (results shown in FIG. 5, wherein the interrogated GSE4922 dataset included 249 tumors of which 211 were ER+). 8% of the ER+ breast cancers were identified as expressing the “24 gene signature” as set forth above. Decoding the clinical details of these samples revealed that this subset of ER+ tumors exhibited a significantly poorer prognosis than tumors that did not express the 24-gene signature. Within this identified data set, tumors with the identified gene signature were lymph node positive 1.5-fold more frequently (45% compared to 30%) than isolates from tumors than gene signature negative tumors. Patients with these tumors also had a 20% decrease in ten-year disease-free survival and these tumors were also more likely to reoccur or metastasize. The 24-gene signature permitted aggressive ER+ tumors to be identified independently of other pathological, histological or other phenotypic basis.

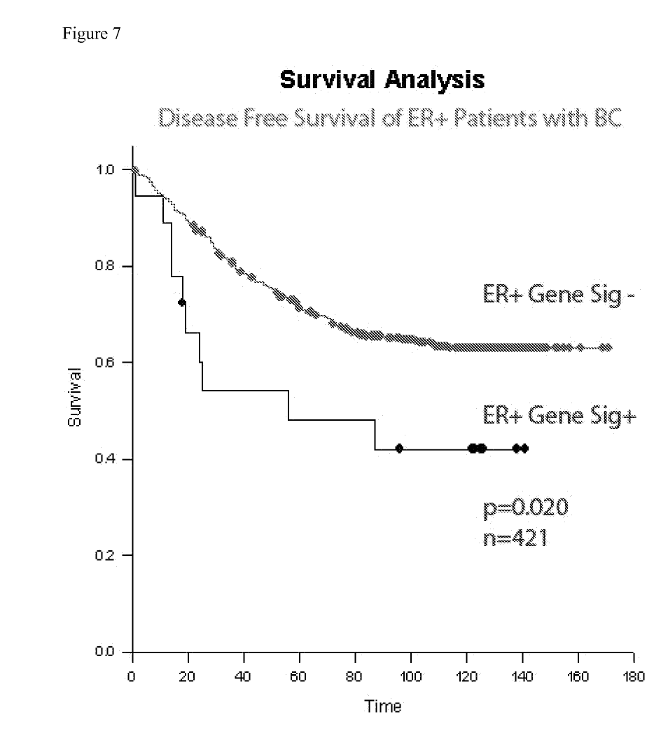

These prognostic data were further verified by performing a survival analysis comparing gene signature positive (GS+), estrogen receptor positive (ER+) breast cancer patients with those ER+ patients that did not express the 24-gene signature (gene-signature negative, or GS−). FIG. 7 shows a graph of “time of disease-free survival following initial diagnosis” for GS+ versus GS− patients. The graph shows that those patients bearing the 24-gene signature had less time until disease recurrence, a result that was statistically significant (having a p value of 0.020 using logrank statistics). At 24 months post-diagnosis, for example, cancer recurred in only 13% of ER+ patients bearing tumors that did not express this gene signature, compared to 40% recurrence in patients bearing tumors that expressed this gene signature. These results showed that detecting expression of the 24-gene signature identified breast cancer patients having a poorer prognosis.

Similar results were obtained from survival analyses performed on 200 ER+ lymph node negative (LN−) tumor samples. At 25 months post-diagnosis, patients with ER+ LN− tumors that did not express the 24-gene signature disclosed herein showed a 14% recurrence rate, compared to a 50% recurrence rate for gene signature-positive tumor samples over the same time interval; these results were also statistically-significant (having a p value of 0.057).

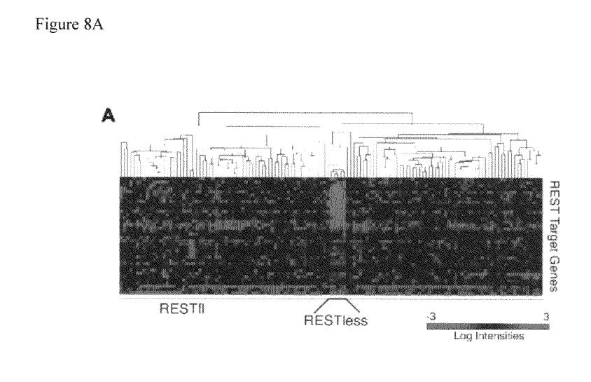

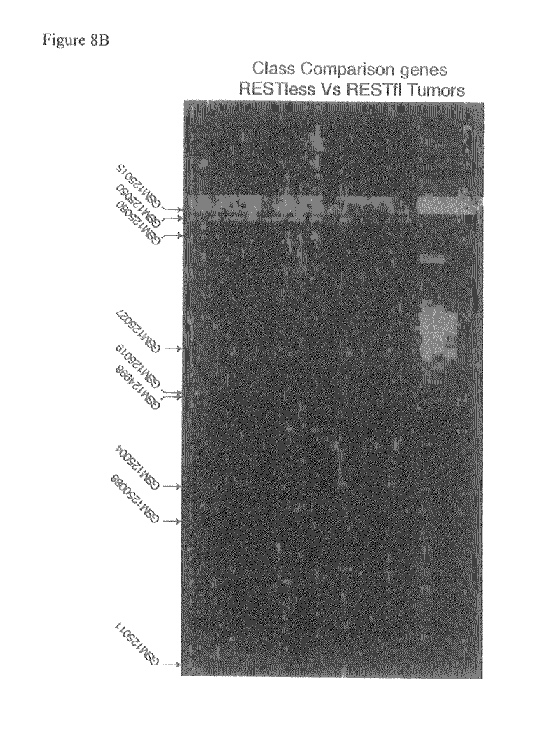

These results were further confirmed in a study using breast cancer tumor set GSE5460, which contains 129 breast cancer tumors. This set of breast cancer tumor samples was interrogated for expression of the 24-gene signature of the invention using microarray screening methods. These results are shown in FIGS. 8A and 8B; microarrays were screened for gene transcripts differentially-expressed between different tumor samples. As with previous tumor collections, a subset of tumors showed overexpression of REST/NRSF target genes.

In additional experiments, microarray analysis performed on yet another breast cancer tumor sample collection showed that expression of several genes was observed to be significantly upregulated; in these experiments, greater than 85% of those genes were established or putative REST/NRSF target genes. Of the 72 genes whose expression has been most closely associated (p<0.0000007) with breast cancer tumors having poorer prognosis and reduced or dysfunctional REST/NRSF expression (RESTless tumors), 63 were upregulated two-fold or greater upon experimental REST/NRSF knockdown, or contained perfect consensus RE1 sites, or were bound by REST/NRSF in a genome-wide ChIP-Seq screen (Johnson, et al., Science 316: 1497-1502), suggesting that these genes are direct targets of REST/NRSF repression (FIG. 8B).

Gene Set Enrichment Analysis (GSEA) was performed on this same subset of breast tumors using the 24-gene signature (FIG. 8C). This method compared the expression of a set of experimentally defined REST/NRSF target genes (termed “S”) between RESTless and RESTfl tumors, and assessed the relative enrichment of S in either tumor group. The positive enrichment score obtained from these analyses, along with the low nominal P-value (p<0.001) and false discovery rate q-value (FDR q-value<0.001), were indicative of high level enrichment of REST/NRSF target gene expression in the RESTless tumor subset. GSEA was also performed using an expanded signature consisting of a list of 92 genes set forth in Table 2 that were at least two-fold over-expressed across the average of all three RESTless cell lines. The results showed that the tumors identified as having poorer prognosis and a greater capacity for growth and metastasis expressed gene signatures of the invention with high statistical significance (nominal p-value<0.001, FDR q-value<0.001). These results confirmed the reliability of the association between detecting altered (increased) expression of the genes in the gene signatures of this invention, particularly as set forth in Tables 1 and 2, and aggressive breast cancer (characterized by poorer prognosis and a greater capacity for growth and metastasis), as well as increasing the association and predictive value of alteration in expression of these genes with absent, reduced or dysfunctional REST/NRSF expression.

Finally, GSEA was also performed using an unbiased list of REST/NRSF targets derived from a ChIPSeq array assay performed in a wholly different cell system, Jurkat T (T cell leukemia) cells (Johnson, et al., Science 316: 1497-1502). ChIPSeq identified REST binding sites in the Jurkat T-cell genome by crosslinking REST to chromatin, fragmenting the REST-crosslinked chromatin and then immunoprecipitating crosslinked fragments with an anti-REST antibody. DNA fragments precipitated with the anti-REST antibody were then de-crosslinked, purified and subjected to direct ultra-high-throughput sequencing to identify REST binding sites. REST target genes identified by this approach were found to be significantly (nominal p-value<0.001, FDR q-value<0.001) enriched in breast cancer tumors identified as having poorer prognosis and a greater capacity for growth and metastasis (FIG. 8C).

A summary of those genes exhibiting aberrant expression in RESTless tumors as compared to RESTfl samples is provided in Table 4. Genes shown to be differentially expressed (i.e., upregulated or downregulated) between RESTless and RESTfl tumors from breast cancer tumor set GSE5460 encompassed 317 genes (Table 4). To summarize, the genes contained in this Table 4 were identified as differentially expressed based on one or more of the following assays: presence of “24 Gene Signature” 2) comparison data showing a plurality of those genes to be REST targets 3) GSEA analysis using multiple genesets and 4) direct identification and measurement of REST4 splicing transcript in 2 of these 5 tumors (shown below).

TABLE 4
Genes differentially regulated (i.e., upregulated or downregulated) in the
absence of functional REST/NRSF in RESTless breast cancer tumor set GSE5460.

| Gene | | Transcript | |
|---|---|---|---|
| Abbrev. | Gene Name | Accession No.* | SEQ ID NO: |
| IFITM1 | Interferon-induced transmembrane | ENST00000328221 | SEQ ID NO: 94 |
| | protein 1 (Interferon-induced protein | | |
| | 17) (Interferon-inducible protein 9- | | |
| | 27) (Leu-13 antigen) (CD225 | | |
| | antigen) | | |
| AGRN | Agrin precursor | ENST00000345038 | SEQ ID NO: 95 |
| CACNA1C | Voltage-dependent L-type calcium | ENST00000327702 | SEQ ID NO: 96 |
| | channel alpha-1C subunit (Voltage- | | |
| | gated calcium channel alpha subunit | | |
| | Cav1.2) (Calcium channel, L type, | | |
| | alpha-1 polypeptide, isoform 1, | | |
| | cardiac muscle) | | |
| CECR6 | Cat eye syndrome critical region | ENST00000331437 | SEQ ID NO: 30 |
| | protein 6 | | |
| GABRD | Gamma-aminobutyric-acid receptor | ENST00000344115 | SEQ ID NO: 34 |
| | delta subunit precursor (GABA(A) | | |
| | receptor) | | |
| CRMP1 | Dihydropyrimidinase related | ENST00000338991 | SEQ ID NO: 97 |
| | protein-1 (DRP-1) (Collapsin | | |
| | response mediator protein 1) | | |
| | (CRMP-1) | | |
| FXC1 | Mitochondrial import inner | ENST00000254616 | SEQ ID NO: 98 |
| | membrane translocase subunit TIM9 | | |
| | B (Fracture callus protein 1) (FxC1) | | |
| | (TIM10B) (TIMM10B) | | |
| DISP2 | dispatched B | ENST00000267889 | SEQ ID NO: 6 |
| ANKRD29 | ankyrin repeat domain 29 | ENST00000322980 | SEQ ID NO: 99 |
| CD69 | Early activation antigen CD69 | ENST00000228434 | SEQ ID NO: 100 |
| | (Early T-cell activation antigen p60) | | |
| | (GP32/28) (Leu-23) (MLR-3) (EA1) | | |
| | (BL-AC/P26) (Activation inducer | | |
| | molecule) (AIM) | | |
| CUGBP2 | CUG triplet repeat, RNA binding | ENST00000354440 | SEQ ID NO: 101 |
| | protein 2 | | |
| KCNJ6 | G protein-activated inward rectifier | ENST00000288309 | SEQ ID NO: 102 |
| | potassium channel 2 (GIRK2) | | |
| | (Potassium channel, inwardly | | |
| | rectifying, subfamily J, member 6) | | |
| | (Inward rectifier K(+) channel | | |
| | Kir3.2) (KATP-2) (BIR1) | | |
| C19orf30 | Chromosome 19 Open reading | ENST00000317292 | SEQ ID NO: 103 |
| | frame 30 | | |
| ABCC8 | Sulfonylurea receptor 1 | ENST00000302539 | SEQ ID NO: 104 |
| KCNC1 | Potassium voltage-gated channel | ENST00000265969 | SEQ ID NO: 105 |
| | subfamily C member 1 (Voltage- | | |
| | gated potassium channel subunit | | |
| | Kv3.1) (Kv4) (NGK2) | | |
| EHD3 | EH-domain containing protein 3 | ENST00000336339 | SEQ ID NO: 106 |
| BRUNOL4 | bruno-like 4, RNA binding protein | ENST00000361795 | SEQ ID NO: 107 |
| LETM2 | leucine zipper-EF-hand containing | ENST00000297720 | SEQ ID NO: 108 |
| | transmembrane protein 2 | | |
| FGFR1 | Basic fibroblast growth factor | ENST00000326324 | SEQ ID NO: 109 |
| | receptor 1 precursor (EC 2.7.1.112) | | |
| | (FGFR-1) (bFGF-R) (Fms-like | | |
| | tyrosine kinase-2) (c-fgr) | | |
| FGFR1 | Basic fibroblast growth factor | ENST00000356207 | SEQ ID NO: 110 |
| | receptor 1 precursor (EC 2.7.1.112) | | |
| | (FGFR-1) (bFGF-R) (Fms-like | | |
| | tyrosine kinase-2) (c-fgr) | | |
| DNAH9 | Ciliary dynein heavy chain 9 | ENST00000262442 | SEQ ID NO: 111 |
| | (Axonemal beta dynein heavy chain | | |
| | 9) | | |
| ANK1 | Ankyrin 1 (Erythrocyte ankyrin) | ENST00000347528 | SEQ ID NO: 112 |
| | (Ankyrin R) | | |
| CHGB | Secretogranin-1 precursor | ENST00000203005 | SEQ ID NO: 3 |
| | (Secretogranin I) (SgI) | | |
| | (Chromogranin B) (CgB) [Contains: | | |
| | GAWK peptide; CCB peptide] | | |
| CAMK2B | Calcium/calmodulin-dependent | ENST00000324091 | SEQ ID NO: 113 |
| | protein kinase type II beta chain (EC | | |
| | 2.7.1.123) (CaM-kinase II beta | | |
| | chain) (CaM kinase II beta subunit) | | |
| | (CaMK-II beta subunit) | | |
| CACNB2 | Voltage-dependent L-type calcium | ENST00000324631 | SEQ ID NO: 114 |
| | channel beta-2 subunit (CAB2) | | |
| | (Calcium channel, voltage- | | |
| | dependent, beta 2 subunit) | | |
| | (Lambert-Eaton myasthenic | | |
| | syndrome antigen B) (MYSB) | | |
| CHGA | Chromogranin A precursor (CgA) | ENST00000216492 | SEQ ID NO: 115 |
| | (Pituitary secretory protein I) (SP-I) | | |
| | [Contains: Vasostatin-1 (Vasostatin | | |
| | I); Vasostatin-2 (Vasostatin II); EA- | | |
| | 92; ES-43; Pancreastatin; SS-18; | | |
| | WA-8; WE-14; LF-19; AL-11; GV- | | |
| | 19; GR-44; ER-37] | | |
| IGFBP3 | Insulin-like growth factor binding | ENST00000275521 | SEQ ID NO: 116 |
| | protein 3 precursor (IGFBP-3) (IBP- | | |
| | 3) (IGF-binding protein 3) | | |
| KIAA1409 | KIAA1409 | ENST00000256339 | SEQ ID NO: 117 |
| IGJ | Immunoglobulin J chain | ENST00000254801 | SEQ ID NO: 118 |
| C9orf25 | Chromosome 9 Open reading frame | ENST00000359556 | SEQ ID NO: 119 |
| | 25 | | |
| GNB5 | Guanine nucleotide-binding protein | ENST00000358784 | SEQ ID NO: 120 |
| | beta subunit 5 (Transducin beta | | |
| | chain 5) (Gbeta5) | | |
| CHST1 | carbohydrate (keratan sulfate Gal-6) | ENST00000308064 | SEQ ID NO: 121 |
| | sulfotransferase 1 | | |
| KIF9 | Kinesin-like protein KIF9 | ENST00000265529 | SEQ ID NO: 122 |
| HLA-C | HLA class I histocompatibility | ENST00000259866 | SEQ ID NO: 123 |
| | antigen, Cw-18 alpha chain | | |
| | precursor (MHC class I antigen | | |
| | Cw*18) | | |
| GPR158 | G protein-coupled receptor 158 | ENST00000280625 | SEQ ID NO: 124 |
| KIAA0329 | | ENST00000359520 | SEQ ID NO: 125 |
| KDELR3 | ER lumen protein retaining receptor | ENST00000216014 | SEQ ID NO: 126 |
| | 3 (KDEL receptor 3) (KDEL | | |
| | endoplasmic reticulum protein | | |
| | retention receptor 3) | | |
| GDAP1 | Ganglioside-induced differentiation- | ENST00000220822 | SEQ ID NO: 87 |
| | associated protein 1 (GDAP1) | | |
| CELSR3 | Cadherin EGF LAG seven-pass G- | ENST00000164024 | SEQ ID NO: 127 |
| | type receptor 3 precursor (Flamingo | | |
| | homolog 1) (hFmi1) (Multiple | | |
| | epidermal growth factor-like | | |
| | domains 2) (Epidermal growth | | |
| | factor-like 1) | | |
| FABP5 | Fatty acid-binding protein, | ENST00000297258 | SEQ ID NO: 128 |
| | epidermal (E-FABP) (Psoriasis- | | |
| | associated fatty acid-binding protein | | |
| | homolog) (PA-FABP) | | |
| INSM1 | Zinc finger protein IA-1 | ENST00000310227 | SEQ ID NO: 129 |
| | (Insulinoma-associated protein 1) | | |
| EGR4 | Early growth response protein 4 | ENST00000258092 | SEQ ID NO: 130 |
| | (EGR-4) (AT133) | | |
| DHPS | Deoxyhypusine synthase (EC | ENST00000210060 | SEQ ID NO: 131 |
| | 2.5.1.46) (DHS) | | |
| EDIL3 | EGF-like repeats and discoidin I-like | ENST00000296591 | SEQ ID NO: 132 |
| | domains protein 3 precursor | | |
| | (Developmentally regulated | | |
| | endothelial cell locus 1 protein) | | |
| | (Integrin-binding protein DEL1) | | |
| IGHG3 | Ig mu chain C region membrane- | ENST00000361286 | SEQ ID NO: 133 |
| | bound segment | | |
| IGHG3 | Ig mu chain C region membrane- | ENST00000300887 | SEQ ID NO: 134 |
| | bound segment | | |
| IGHG3 | Ig mu chain C region membrane- | ENST00000343496 | SEQ ID NO: 135 |
| | bound segment | | |
| IGHG3 | Ig mu chain C region membrane- | ENST00000251006 | SEQ ID NO: 136 |
| | bound segment | | |
| IGHG3 | Ig mu chain C region membrane- | ENST00000361266 | SEQ ID NO: 137 |
| | bound segment | | |
| FABP5 | Fatty acid-binding protein, | ENST00000300149 | SEQ ID NO: 138 |
| | epidermal (E-FABP) (Psoriasis- | | |
| | associated fatty acid-binding protein | | |
| | homolog) (PA-FABP) | | |
| CACNA2D2 | calcium channel, voltage-dependent, | ENST00000360963 | SEQ ID NO: 139 |
| | alpha 2/delta subunit 2 isoform a | | |
| IGKC | Ig kappa chain V-I region Walker | ENST00000334308 | SEQ ID NO: 140 |
| | precursor | | |
| IGKC | Ig kappa chain V-I region Walker | ENST00000303153 | SEQ ID NO: 141 |
| | precursor | | |
| IGKV4-1 | Ig kappa chain V-IV region | ENST00000283657 | SEQ ID NO: 142 |
| | precursor (Fragment) | | |
| GRM4 | Metabotropic glutamate receptor 4 | ENST00000266007 | SEQ ID NO: 143 |
| | precursor (mGluR4) | | |
| ALPK1 | alpha-kinase 1 | ENST00000177648 | SEQ ID NO: 144 |
| CAMK2D | Calcium/calmodulin-dependent | ENST00000342666 | SEQ ID NO: 145 |
| | protein kinase type II delta chain | | |
| | (EC 2.7.1.123) (CaM-kinase II delta | | |
| | chain) (CaM kinase II delta subunit) | | |
| | (CaMK-II delta subunit) | | |
| KCTD6 | potassium channel tetramerisation | ENST00000355076 | SEQ ID NO: 146 |
| | domain containing 6 | | |
| KCTD6 | potassium channel tetramerisation | ENST00000302803 | SEQ ID NO: 147 |
| | domain containing 6 | | |
| ACD | adrenocortical dysplasia homolog | ENST00000219251 | SEQ ID NO: 148 |
| ATP6V0A1 | Vacuolar proton translocating | ENST00000343619 | SEQ ID NO: 149 |
| | ATPase 116 kDa subunit a isoform 1 | | |
| | (V-ATPase 116-kDa isoform a1) | | |
| | (Clathrin-coated vesicle/synaptic | | |
| | vesicle proton pump 116 kDa | | |
| | subunit) (Vacuolar proton pump | | |
| | subunit 1) (Vacuolar adenosine | | |
| | triphosphatase subunit Ac116) | | |
| GRIA2 | Glutamate receptor 2 precursor | ENST00000264426 | SEQ ID NO: 150 |
| | (GluR-2) (GluR-B) (GluR-K2) | | |
| | (Glutamate receptor ionotropic, | | |
| | AMPA 2) | | |
| CD47 | Leukocyte surface antigen CD47 | ENST00000361309 | SEQ ID NO: 151 |
| | precursor (Antigenic surface | | |
| | determinant protein OA3) (Integrin | | |
| | associated protein) (IAP) (MER6) | | |
| KCNC2 | Shaw-related voltage-gated | ENST00000341669 | SEQ ID NO: 152 |
| | potassium channel protein 2 isoform | | |
| | KV3.2c | | |
| APLP1 | Amyloid-like protein 1 precursor | ENST00000221891 | SEQ ID NO: 153 |
| | (APLP) (APLP-1) [Contains: C30] | | |
| DMRTC1 | DMRT-like family C1 | ENST00000334036 | SEQ ID NO: 154 |
| DMRTC1 | DMRT-like family C1 | ENST00000334472 | SEQ ID NO: 155 |
| GPM6A | Neuronal membrane glycoprotein | ENST00000280187 | SEQ ID NO: 156 |
| | M6-a (M6a) | | |
| GPM6A | Neuronal membrane glycoprotein | ENST00000359631 | SEQ ID NO: 157 |
| | M6-a (M6a) | | |
| DPYSL3 | Dihydropyrimidinase related | ENST00000343218 | SEQ ID NO: 158 |
| | protein-3 (DRP-3) (Unc-33-like | | |
| | phosphoprotein) (ULIP protein) | | |
| | (Collapsin response mediator protein | | |
| | 4) (CRMP-4) | | |
| KCNJ3 | G protein-activated inward rectifier | ENST00000295101 | SEQ ID NO: 159 |
| | potassium channel 1 (GIRK1) | | |
| | (Potassium channel, inwardly | | |
| | rectifying, subfamily J, member 3) | | |
| | (Inward rectifier K(+) channel | | |
| | Kir3.1) | | |
| GRIA1 | Glutamate receptor 1 precursor | ENST00000285900 | SEQ ID NO: 160 |
| | (GluR-1) (GluR-A) (GluR-K1) | | |
| | (Glutamate receptor ionotropic, | | |
| | AMPA 1) | | |
| CHPT1 | choline phosphotransferase 1 | ENST00000229266 | SEQ ID NO: 161 |
| ASCL1 | Achaete-scute homolog 1 (HASH1) | ENST00000266744 | SEQ ID NO: 162 |
| CEACAM5 | Carcinoembryonic antigen-related | ENST00000221992 | SEQ ID NO: 163 |
| | cell adhesion molecule 5 precursor | | |
| | (Carcinoembryonic antigen) (CEA) | | |
| | (Meconium antigen 100) (CD66e | | |
| | antigen) | | |
| BEX1 | brain expressed, X-linked 1 | ENST00000255533 | SEQ ID NO: 26 |
| KCNH2 | Potassium voltage-gated channel | ENST00000262186 | SEQ ID NO: 164 |
| | subfamily H member 2 (Voltage- | | |
| | gated potassium channel subunit | | |
| | Kv11.1) (Ether-a-go-go related gene | | |
| | potassium channel 1) (H-ERG) | | |
| | (Erg1) (Ether-a-go-go related | | |
| | protein 1) (Eag related protein 1) | | |
| | (eag homolog) | | |
| CPNE4 | Copine IV (Copine 8) | ENST00000357965 | SEQ ID NO: 65 |
| CPNE4 | Copine IV (Copine 8) | ENST00000354260 | SEQ ID NO: 66 |
| CPNE4 | Copine IV (Copine 8) | ENST00000356700 | SEQ ID NO: 67 |
| ATP2A2 | Sarcoplasmic/endoplasmic reticulum | ENST00000313432 | SEQ ID NO: 165 |
| | calcium ATPase 2 (EC 3.6.3.8) | | |
| | (Calcium pump 2) (SERCA2) (SR | | |
| | Ca(2+)-ATPase 2) (Calcium- | | |
| | transporting ATPase sarcoplasmic | | |
| | reticulum type, slow twitch skeletal | | |
| | muscle isoform) (Endoplasmic | | |
| | reticulum class 1/2 Ca(2+) ATPase) | | |
| ALDH2 | Aldehyde dehydrogenase, | ENST00000261733 | SEQ ID NO: 166 |
| | mitochondrial precursor (EC 1.2.1.3) | | |
| | (ALDH class 2) (ALDHI) (ALDH- | | |
| | E2) | | |
| INPP1 | Inositol polyphosphate 1- | ENST00000322522 | SEQ ID NO: 167 |
| | phosphatase (EC 3.1.3.57) (IPPase) | | |
| | (IPP) | | |
| CPB1 | Carboxypeptidase B precursor (EC | ENST00000282957 | SEQ ID NO: 168 |
| | 3.4.17.2) (Pancreas-specific protein) | | |
| | (PASP) | | |
| CA11 | Carbonic anhydrase-related protein | ENST00000084798 | SEQ ID NO: 169 |
| | 2 precursor (CARP-2) (CA-RP II) | | |
| | (CA-XI) (Carbonic anhydrase- | | |
| | related protein 11) (CARP XI) (CA- | | |
| | RP XI) (UNQ211/PRO237) | | |
| BCL2L12 | Bcl-2 related proline-rich protein | ENST00000246785 | SEQ ID NO: 170 |
| | (Bcl-2-like 12 protein) | | |
| ECT2 | ECT2 protein (Epithelial cell | ENST00000232458 | SEQ ID NO: 171 |
| | transforming sequence 2 oncogene) | | |
| EEF1A2 | Elongation factor 1-alpha 2 (EF-1- | ENST00000217182 | SEQ ID NO: 172 |
| | alpha-2) (Elongation factor 1 A-2) | | |
| | (eEF1A-2) (Statin S1) | | |
| L1CAM | Neural cell adhesion molecule L1 | ENST00000361699 | SEQ ID NO: 173 |
| | precursor (N-CAM L1) (CD171 | | |
| | antigen) | | |
| DNAJC6 | DnaJ (Hsp40) homolog, subfamily | ENST00000263441 | SEQ ID NO: 174 |
| | C, member 6 | | |
| HS2ST1 | heparan sulfate 2-O-sulfotransferase 1 | ENST00000284064 | SEQ ID NO: 175 |
| CNN3 | Calponin-3 (Calponin, acidic | ENST00000281863 | SEQ ID NO: 176 |
| | isoform) | | |
| ATRNL1 | attractin-like 1 | ENST00000355044 | SEQ ID NO: 177 |
| ATRNL1 | attractin-like 1 | ENST00000303745 | SEQ ID NO: 178 |
| DPYSL4 | Dihydropyrimidinase related | ENST00000338492 | SEQ ID NO: 179 |
| | protein-4 (DRP-4) (Collapsin | | |
| | response mediator protein 3) | | |
| | (CRMP-3) (UNC33-like | | |
| | phosphoprotein 4) (ULIP4 protein) | | |
| EFNA4 | Ephrin-A4 precursor (EPH-related | ENST00000271938 | SEQ ID NO: 180 |
| | receptor tyrosine kinase ligand 4) | | |
| | (LERK-4) | | |
| FAM20B | Protein FAM20B precursor | ENST00000263733 | SEQ ID NO: 181 |
| CHI3L1 | Chitinase-3 like protein 1 precursor | ENST00000255409 | SEQ ID NO: 182 |
| | (Cartilage glycoprotein-39) (GP-39) | | |
| | (39 kDa synovial protein) (HCgp- | | |
| | 39) (YKL-40) | | |
| GNG4 | Guanine nucleotide-binding protein | ENST00000302505 | SEQ ID NO: 36 |
| | G(I)/G(S)/G(O) gamma-4 subunit | | |
| CNR1 | Cannabinoid receptor 1 (CB1) (CB- | ENST00000303726 | SEQ ID NO: 183 |
| | R) (CANN6) | | |
| SLC22A17 | Brain-type organic cation transporter | ENST00000206544 | SEQ ID NO: 184 |
| | (Solute carrier family 22, member | | |
| | 17) | | |
| NOVA1 | RNA-binding protein Nova-1 | ENST00000267422 | SEQ ID NO: 185 |
| | (Neuro-oncological ventral antigen | | |
| | 1) (Onconeural ventral antigen-1) | | |
| | (Paraneoplastic Ri antigen) (Ventral | | |
| | neuron-specific protein 1) | | |
| POLE2 | DNA polymerase epsilon subunit B | ENST00000216367 | SEQ ID NO: 186 |
| | (EC 2.7.7.7) (DNA polymerase II | | |
| | subunit B) | | |
| TRIM9 | Tripartite motif protein 9 (RING | ENST00000298355 | SEQ ID NO: 93 |
| | finger protein 91) | | |
| USP25 | Ubiquitin carboxyl-terminal | ENST00000285681 | SEQ ID NO: 187 |
| | hydrolase 25 (EC 3.1.2.15) | | |
| | (Ubiquitin thiolesterase 25) | | |
| | (Ubiquitin-specific processing | | |
| | protease 25) (Deubiquitinating | | |
| | enzyme 25) (USP on chromosome | | |
| | 21) | | |
| NET1 | Neuroepithelial cell transforming | ENST00000308281 | SEQ ID NO: 188 |
| | gene 1 protein (p65 Net1 proto- | | |
| | oncogene) (Rho guanine nucleotide | | |
| | exchange factor 8) | | |
| NTSR2 | Neurotensin receptor type 2 (NT-R- | ENST00000306928 | SEQ ID NO: 189 |
| | 2) (Levocabastine-sensitive | | |
| | neurotensin receptor) (NTR2 | | |
| | receptor) | | |
| NTN2L | Netrin-2 like protein precursor | ENST00000293973 | SEQ ID NO: 190 |
| USP6NL | USP6 N-terminal like protein | ENST00000277575 | SEQ ID NO: 191 |
| | (Related to the N terminus of tre) | | |
| | (RN-tre) | | |
| QDPR | Dihydropteridine reductase (EC | ENST00000281243 | SEQ ID NO: 192 |
| | 1.5.1.34) (HDHPR) (Quinoid | | |
| | dihydropteridine reductase) | | |
| MAPRE3 | Microtubule-associated protein | ENST00000233121 | SEQ ID NO: 193 |
| | RP/EB family member 3 (End- | | |
| | binding protein 3) (EB3) (EB1 | | |
| | protein family member 3) (EBF3) | | |
| | (RP3) | | |
| SLC5A6 | Sodium-dependent multivitamin | ENST00000310574 | SEQ ID NO: 194 |
| | transporter (Na(+)-dependent | | |
| | multivitamin transporter) | | |
| PTGER4 | Prostaglandin E2 receptor, EP4 | ENST00000302472 | SEQ ID NO: 195 |
| | subtype (Prostanoid EP4 receptor) | | |
| | (PGE receptor, EP4 subtype) | | |
| RIPK4 | Serine/threonine-protein kinase | ENST00000352483 | SEQ ID NO: 196 |
| | RIPK4 (EC 2.7.1.37) (Receptor- | | |
| | interacting serine-threonine kinase | | |
| | 4) (Ankyrin repeat domain protein | | |
| | 3) (PKC-delta-interacting protein | | |
| | kinase) | | |
| SEZ6L | Seizure 6-like protein precursor | ENST00000248933 | SEQ ID NO: 197 |
| NOL4 | Nucleolar protein 4 (Nucleolar- | ENST00000261592 | SEQ ID NO: 198 |
| | localized protein) (HRIHFB2255) | | |
| TPH1 | Tryptophan 5-hydroxylase 1 (EC | ENST00000250018 | SEQ ID NO: 199 |
| | 1.14.16.4) (Tryptophan 5- | | |
| | monooxygenase 1) | | |
| NEFH | Neurofilament triplet H protein (200 kDa | ENST00000310624 | SEQ ID NO: 200 |
| | neurofilament protein) | | |
| | (Neurofilament heavy polypeptide) | | |
| | (NF-H) | | |
| TSG101 | Tumor susceptibility gene 101 | ENST00000251968 | SEQ ID NO: 201 |
| | protein | | |
| SYT4 | Synaptotagmin-4 (Synaptotagmin | ENST00000255224 | SEQ ID NO: 202 |
| | IV) (SytIV) | | |
| SCGN | Secretagogin | ENST00000190668 | SEQ ID NO: 203 |
| NRXN3 | Neurexin 3-alpha precursor | ENST00000330071 | SEQ ID NO: 204 |
| | (Neurexin III-alpha) | | |
| SEMA6D | semaphorin 6D isoform 6 precursor | ENST00000316364 | SEQ ID NO: 205 |
| GABBR1 | Gamma-aminobutyric acid type B | ENST00000259937 | SEQ ID NO: 206 |
| | receptor, subunit 1 precursor | | |
| | (GABA-B receptor 1) (GABA-B- | | |
| | R1) (Gb1) | | |
| SCG3 | Secretogranin-3 precursor | ENST00000220478 | SEQ ID NO: 207 |
| | (Secretogranin III) (SgIII) | | |
| | (UNQ2502/PRO5990) | | |
| SLC4A4 | solute carrier family 4, sodium | ENST00000264485 | SEQ ID NO: 208 |
| | bicarbonate cotransporter, member 4 | | |
| SYTL5 | Synaptotagmin-like protein 5 | ENST00000357972 | SEQ ID NO: 209 |
| SYTL5 | Synaptotagmin-like protein 5 | ENST00000297875 | SEQ ID NO: 210 |
| TRPA1 | Transient receptor potential cation | ENST00000262209 | SEQ ID NO: 211 |
| | channel subfamily A member 1 | | |
| | (Ankyrin-like with transmembrane | | |
| | domains protein 1) (Transformation | | |
| | sensitive-protein p120) | | |
| MADD | MAP-kinase activating death | ENST00000311027 | SEQ ID NO: 212 |
| | domain-containing protein isoform g | | |
| SNX5 | Sorting nexin 5 | ENST00000341703 | SEQ ID NO: 213 |
| STX1A | Syntaxin-1A (Neuron-specific | ENST00000222812 | SEQ ID NO: 56 |
| | antigen HPC-1) | | |
| NAPB | Beta-soluble NSF attachment | ENST00000246011 | SEQ ID NO: 214 |
| | protein (SNAP-beta) (N- | | |
| | ethylmaleimide-sensitive factor | | |
| | attachment protein, beta) | | |
| SEZ6L2 | seizure related 6 homolog (mouse)- | ENST00000350527 | SEQ ID NO: 81 |
| | like 2 | | |
| SYN1 | Synapsin-1 (Synapsin I) (Brain | ENST00000263237 | SEQ ID NO: 57 |
| | protein 4.1) | | |
| PCSK1 | Neuroendocrine convertase 1 | ENST00000311106 | SEQ ID NO: 215 |
| | precursor (EC 3.4.21.93) (NEC 1) | | |
| | (PC1) (Prohormone convertase 1) | | |
| | (Proprotein convertase 1) | | |
| PCLO | Piccolo protein (Aczonin) | ENST00000333891 | SEQ ID NO: 216 |
| RIMS2 | Regulating synaptic membrane | ENST00000329869 | SEQ ID NO: 217 |
| | exocytosis protein 2 (Rab3- | | |
| | interacting molecule 2) (RIM 2) | | |
| SYT7 | Synaptotagmin-7 (Synaptotagmin | ENST00000263846 | SEQ ID NO: 218 |
| | VII) (SytVII) | | |
| PARP6 | poly (ADP-ribose) polymerase | ENST00000287196 | SEQ ID NO: 219 |
| | family, member 6 | | |
| SYP | Synaptophysin (Major synaptic | ENST00000263233 | SEQ ID NO: 21 |
| | vesicle protein p38) | | |
| TNFAIP8 | tumor necrosis factor, alpha-induced | ENST00000274456 | SEQ ID NO: 220 |
| | protein 8 | | |
| MAPK8IP2 | C-jun-amino-terminal kinase | ENST00000329492 | SEQ ID NO: 11 |
| | interacting protein 2 (JNK- | | |
| | interacting protein 2) (JIP-2) (JNK | | |
| | MAP kinase scaffold protein 2) | | |
| | (Islet-brain-2) (IB-2) (Mitogen- | | |
| | activated protein kinase 8- | | |
| | interacting protein 2) | | |
| UNC13A | Unc-13 homolog A (Munc13-1) | ENST00000252773 | SEQ ID NO: 221 |
| | (Fragment) | | |
| RAB3A | Ras-related protein Rab-3A | ENST00000222256 | SEQ ID NO: 222 |
| PMS2L8 | PREDICTED: similar to PMS4 | ENST00000222396 | SEQ ID NO: 223 |
| | homolog mismatch repair protein- | | |
| | human | | |
| MCF2L | Guanine nucleotide exchange factor | ENST00000347957 | SEQ ID NO: 224 |
| | DBS (DBL's big sister) (MCF2 | | |
| | transforming sequence-like protein) | | |
| | (Fragment) | | |
| PDE8A | High-affinity cAMP-specific and | ENST00000310298 | SEQ ID NO: 225 |
| | IBMX-insensitive 3′,5′-cyclic | | |
| | phosphodiesterase 8A (EC 3.1.4.17) | | |
| ROBO2 | Roundabout homolog 2 precursor | ENST00000332191 | SEQ ID NO: 226 |
| RASA4 | Ras GTPase-activating protein 4 | ENST00000306682 | SEQ ID NO: 227 |
| | (RasGAP-activating-like protein 2) | | |
| | (Calcium-promoted Ras inactivator) | | |
| ERCC6 | DNA excision repair protein ERCC- | ENST00000342592 | SEQ ID NO: 228 |
| | 6 (Cockayne syndrome protein CSB) | | |
| RASA4 | Ras GTPase-activating protein 4 | ENST00000262940 | SEQ ID NO: 229 |
| | (RasGAP-activating-like protein 2) | | |
| | (Calcium-promoted Ras inactivator) | | |
| PARD6A | Partitioning defective-6 homolog | ENST00000219255 | SEQ ID NO: 230 |
| | alpha (PAR-6 alpha) (PAR-6A) | | |
| | (PAR-6) (PAR6C) (Tax interaction | | |
| | protein 40) (TIP-40) | | |
| OGDHL | oxoglutarate dehydrogenase-like | ENST00000355036 | SEQ ID NO: 76 |
| SMPD3 | sphingomyelin phosphodiesterase 3, | ENST00000219334 | SEQ ID NO: 54 |
| | neutral membrane | | |
| SCN1B | Sodium channel beta-1 subunit | ENST00000262631 | SEQ ID NO: 231 |
| | precursor | | |
| NPY5R | Neuropeptide Y receptor type 5 | ENST00000338566 | SEQ ID NO: 232 |
| | (NPY5-R) (NPY-Y5 receptor) (Y5 | | |
| | receptor) (NPYY5) | | |
| NRBF2 | nuclear receptor binding factor 2 | ENST00000277746 | SEQ ID NO: 233 |
| PCDHAC2 | Protocadherin alpha 13 precursor | ENST00000289630 | SEQ ID NO: 234 |
| | (PCDH-alpha13) | | |
| PCDHB3 | Protocadherin beta 3 precursor | ENST00000231130 | SEQ ID NO: 235 |
| | (PCDH-beta3) | | |
| NTS | Neurotensin/neuromedin N | ENST00000256010 | SEQ ID NO: 236 |
| | precursor [Contains: Large | | |
| | neuromedin N (NmN-125); | | |
| | Neuromedin N (NmN) (NN); | | |
| | Neurotensin (NT); Tail peptide] | | |
| WDR17 | WD-repeat protein 17 | ENST00000280190 | SEQ ID NO: 237 |
| TERF2IP | Telomeric repeat binding factor 2 | ENST00000300086 | SEQ ID NO: 238 |
| | interacting protein 1 (TRF2- | | |
| | interacting telomeric protein Rap1) | | |
| | (hRap1) | | |
| PODXL | Podocalyxin-like protein 1 precursor | ENST00000322985 | SEQ ID NO: 239 |
| RIMS4 | Regulating synaptic membrane | ENST00000217067 | SEQ ID NO: 240 |
| | exocytosis protein 4 (Rab-3 | | |
| | interacting molecule 4) (RIM 4) | | |
| | (RIM4 gamma) | | |
| PODXL2 | endoglycan | ENST00000342480 | SEQ ID NO: 241 |
| MGLL | Monoglyceride lipase (EC 3.1.1.23) | ENST00000265052 | SEQ ID NO: 242 |
| | (HU-K5) (Lysophospholipase | | |
| | homolog) (Lysophospholipase-like) | | |
| LRP2 | Low-density lipoprotein receptor- | ENST00000263816 | SEQ ID NO: 243 |
| | related protein 2 precursor (Megalin) | | |
| | (Glycoprotein 330) (gp330) | | |
| TMEM22 | transmembrane protein 22 | ENST00000306215 | SEQ ID NO: 244 |
| PPM1E | protein phosphatase 1E | ENST00000308249 | SEQ ID NO: 245 |
| PTPRN2 | Receptor-type tyrosine-protein | ENST00000331938 | SEQ ID NO: 246 |
| | phosphatase N2 precursor (EC | | |
| | 3.1.3.48) (R-PTP-N2) (Islet cell | | |
| | autoantigen related protein) | | |
| | (ICAAR) (IAR) (Phogrin) | | |
| UBE2E3 | Ubiquitin-conjugating enzyme E2 | ENST00000305934 | SEQ ID NO: 247 |
| | E3 (EC 6.3.2.19) (Ubiquitin-protein | | |
| | ligase E3) (Ubiquitin carrier protein | | |
| | E3) (Ubiquitin-conjugating enzyme | | |
| | E2-23 kDa) (UbcH9) | | |
| PAPPA | Pappalysin-1 precursor (EC | ENST00000328252 | SEQ ID NO: 248 |
| | 3.4.24.79) (Pregnancy-associated | | |
| | plasma protein-A) (PAPP-A) | | |
| | (Insulin-like growth factor- | | |
| | dependent IGF binding protein-4 | | |
| | protease) (IGF-dependent IGFBP-4 | | |
| | protease) (IGFBP-4ase) | | |
| RAB23 | Ras-related protein Rab-23 | ENST00000317483 | SEQ ID NO: 249 |
| | (HSPC137) | | |
| RAB23 | Ras-related protein Rab-23 | ENST00000344445 | SEQ ID NO: 250 |
| | (HSPC137) | | |
| PPFIA3 | Liprin-alpha 3 (Protein tyrosine | ENST00000334186 | SEQ ID NO: 251 |
| | phosphatase receptor type f | | |
| | polypeptide-interacting protein alpha | | |
| | 3) (PTPRF-interacting protein alpha | | |
| | 3) | | |
| TEAD2 | Transcriptional enhancer factor | ENST00000311227 | SEQ ID NO: 252 |
| | TEF-4 (TEA domain family member | | |
| | 2) (TEAD-2) | | |
| SOX9 | Transcription factor SOX-9 | ENST00000245479 | SEQ ID NO: 253 |
| IL4I1 | Nuclear pore glycoprotein p62 (62 kDa | ENST00000352066 | SEQ ID NO: 254 |
| | nucleoporin) | | |
| IL4I1 | Nuclear pore glycoprotein p62 (62 kDa | ENST00000345498 | SEQ ID NO: 255 |
| | nucleoporin) | | |
| SLC15A4 | solute carrier family 15, member 4 | ENST00000266771 | SEQ ID NO: 256 |
| STMN3 | Stathmin 3 (SCG10-like protein) | ENST00000358145 | SEQ ID NO: 20 |
| PLCD4 | phospholipase C, delta 4 | ENST00000251959 | SEQ ID NO: 257 |
| MAGEA12 | Melanoma-associated antigen 12 | ENST00000276344 | SEQ ID NO: 258 |
| | (MAGE-12 antigen) (MAGE12F) | | |
| SCG2 | Secretogranin-2 precursor | ENST00000305409 | SEQ ID NO: 259 |
| | (Secretogranin II) (SgII) | | |
| | (Chromogranin C) [Contains: | | |
| | Secretoneurin (SN)] | | |
| TFRC | Transferrin receptor protein 1 | ENST00000360110 | SEQ ID NO: 260 |
| | (TfR1) (TR) (TfR) (Trfr) (CD71 | | |
| | antigen) (T9) (p90) | | |
| TFRC | Transferrin receptor protein 1 | ENST00000265238 | SEQ ID NO: 261 |
| | (TfR1) (TR) (TfR) (Trfr) (CD71 | | |
| | antigen) (T9) (p90) | | |
| RAB39B | Ras-related protein Rab-39B | ENST00000286430 | SEQ ID NO: 262 |
| TSPYL4 | Testis-specific Y-encoded-like | ENST00000336786 | SEQ ID NO: 263 |
| | protein 4 (TSPY-like 4) | | |
| PDE4B | cAMP-specific 3′,5′-cyclic | ENST00000329654 | SEQ ID NO: 264 |
| | phosphodiesterase 4B (EC 3.1.4.17) | | |
| | (DPDE4) (PDE32) | | |
| PIGK | GPI-anchor transamidase precursor | ENST00000271047 | SEQ ID NO: 265 |
| | (EC 3.—.—.—) (GPI transamidase) | | |
| | (Phosphatidylinositol-glycan | | |
| | biosynthesis, class K protein) (PIG- | | |
| | K) (hGPI8) | | |
| PERP | PERP, TP53 apoptosis effector | ENST00000265603 | SEQ ID NO: 266 |
| TCF7L2 | Transcription factor 7-like 2 (HMG | ENST00000355717 | SEQ ID NO: 267 |
| | box transcription factor 4) (T-cell- | | |
| | specific transcription factor 4) (TCF- | | |
| | 4) (hTCF-4) | | |
| QKI | quaking homolog, KH domain RNA | ENST00000361752 | SEQ ID NO: 268 |
| | binding isoform HQK-5 | | |
| MCL1 | Induced myeloid leukemia cell | ENST00000271648 | SEQ ID NO: 269 |
| | differentiation protein Mcl-1 | | |
| NMNAT2 | Nicotinamide mononucleotide | ENST00000287713 | SEQ ID NO: 270 |
| | adenylyltransferase 2 (EC 2.7.7.1) | | |
| | (NMN adenylyltransferase 2) | | |
| RGS1 | Regulator of G-protein signaling 1 | ENST00000204113 | SEQ ID NO: 271 |
| | (RGS1) (Early response protein | | |
| | 1R20) (B-cell activation protein | | |
| | BL34) | | |
| NAV1 | neuron navigator 1 | ENST00000358222 | SEQ ID NO: 272 |
| RAB7L1 | Ras-related protein Rab-7L1 (Rab-7 | ENST00000235932 | SEQ ID NO: 273 |
| | like protein 1) | | |
| RGS7 | Regulator of G-protein signaling 7 | ENST00000331110 | SEQ ID NO: 274 |
| | (RGS7) | | |
| YES1 | Proto-oncogene tyrosine-protein | ENST00000314574 | SEQ ID NO: 275 |
| | kinase YES (EC 2.7.1.112) (p61- | | |
| | YES) (C-YES) | | |
| ZFP36L1 | Butyrate response factor 1 (TIS11B | ENST00000336440 | SEQ ID NO: 276 |
| | protein) (EGF-response factor 1) | | |
| | (ERF-1) | | |
| ZCWPW1 | Zinc finger CW-type PWWP | ENST00000358428 | SEQ ID NO: 277 |
| | domain protein 1 | | |
| YAP1 | 65 kDa Yes-associated protein | ENST00000345877 | SEQ ID NO: 278 |
| | (YAP65) | | |
| APCDD1L | Homo sapiens adenomatosis | BC101758 | SEQ ID NO: 279 |
| | polyposis coli down-regulated 1-like | | |
| | (cDNA clone MGC: 126807 | | |
| | IMAGE: 8069264), complete cds | | |
| ARL4C | Homo sapiens ADP-ribosylation | NM_005737 | SEQ ID NO: 280 |
| | factor-like 4C | | |
| ATG9B | Homo sapiens ATG9 autophagy | NM_173681 | SEQ ID NO: 281 |
| | related 9 homolog B (S cerevisiae) | | |
| GOLGA7B | Homo sapiens Golgi autoantigen, | NM_001010917 | SEQ ID NO: 282 |
| | golgin subfamily a, 7B | | |
| C12orf34 | Homo sapiens chromosome 12 open | NM_032829 | SEQ ID NO: 283 |
| | reading frame 34 | | |
| C16orf57 | Homo sapiens chromosome 16 open | NM_024598 | SEQ ID NO: 284 |
| | reading frame 57 | | |
| C1orf173 | Homo sapiens chromosome 1 open | NM_001002912 | SEQ ID NO: 285 |
| | reading frame 173 | | |
| CADM2 | Homo sapiens cell adhesion | NM_153184 | SEQ ID NO: 286 |
| | molecule 2 | | |
| CADPS | Homo sapiens Ca++-dependent | NM_003716 | SEQ ID NO: 287 |
| | secretion activator, transcript variant 1 | | |
| CALM1 | Homo sapiens calmodulin 1 | NM_006888 | SEQ ID NO: 288 |
| | (phosphorylase kinase, delta) | | |
| CAMK2N2 | Homo sapiens calcium/calmodulin- | NM_033259 | SEQ ID NO: 29 |
| | dependent protein kinase II inhibitor 2 | | |
| CARTPT | Homo sapiens CART prepropeptide | NM_004291 | SEQ ID NO: 289 |
| CCDC109B | Homo sapiens coiled-coil domain | NM_017918 | SEQ ID NO: 290 |
| | containing 109B | | |
| CCDC64 | Homo sapiens coiled-coil domain | NM_207311 | SEQ ID NO: 291 |
| | containing 64 | | |
| CD55 | Homo sapiens CD55 molecule, | NM_001114752 | SEQ ID NO: 292 |
| | decay accelerating factor for | | |
| | complement (Cromer blood group), | | |
| | transcript variant 2 | | |
| CKMT1B | Homo sapiens creatine kinase, | NM_020990 | SEQ ID NO: 84 |
| | mitochondrial 1B, nuclear gene | | |
| | encoding mitochondrial protein | | |
| CMIP | Homo sapiens c-Maf-inducing | NM_198390 | SEQ ID NO: 293 |
| | protein, transcript variant C-mip | | |
| COQ10A | Homo sapiens coenzyme Q10 | NM_144576 | SEQ ID NO: 294 |
| | homolog A (Scerevisiae), transcript | | |
| | variant 1 | | |
| CPLX2 | Homo sapiens complexin 2, | NM_006650 | SEQ ID NO: 5 |
| | transcript variant 1 | | |
| CYFIP2 | Homo sapiens cytoplasmic FMR1 | NM_001037333 | SEQ ID NO: 295 |
| | interacting protein 2, transcript | | |
| | variant 1 | | |
| EFR3B | Homo sapiens EFR3 homolog B | NM_014971 | SEQ ID NO: 296 |
| | (Scerevisiae) | | |
| EID1 | Homo sapiens EP300 interacting | NM_014335 | SEQ ID NO: 297 |
| | inhibitor of differentiation 1 | | |
| FAM107B | Homo sapiens family with sequence | NM_031453 | SEQ ID NO: 298 |
| | similarity 107, member B | | |
| FAM171B | Homo sapiens family with sequence | NM_177454 | SEQ ID NO: 299 |
| | similarity 171, member B | | |
| FKBP1B | Homo sapiens FK506 binding | NM_054033 | SEQ ID NO: 300 |
| | protein 1B, 12.6 kDa, transcript | | |
| | variant 2 | | |
| MFSD6 | Homo sapiens major facilitator | NM_017694 | SEQ ID NO: 301 |
| | superfamily domain containing 6 | | |
| FLJ23834 | Homo sapiens hypothetical protein | NM_152750 | SEQ ID NO: 302 |
| | FLJ23834 | | |
| FLJ37078 | Homo sapiens hypothetical protein | NM_001110199 | SEQ ID NO: 303 |
| | FLJ37078 | | |
| FOXO6 | PREDICTED: Homo sapiens | XM_002342102 | SEQ ID NO: 304 |
| | forkhead box protein O6 | | |
| FREQ | Homo sapiens frequenin homolog | NM_014286 | SEQ ID NO: 305 |
| | (Drosophila), transcript variant 1 | | |
| GABARAPL2 | Homo sapiens GABA(A) receptor- | NM_007285 | SEQ ID NO: 306 |
| | associated protein-like 2 | | |
| GDI1 | Homo sapiens GDP dissociation | NM_001493 | SEQ ID NO: 307 |
| | inhibitor 1 | | |
| GNAS | Homo sapiens GNAS complex | NM_016592 | SEQ ID NO: 308 |
| | locus, transcript variant 4 | | |
| GPER | Homo sapiens G protein-coupled | NM_001039966 | SEQ ID NO: 309 |
| | estrogen receptor 1, transcript | | |
| | variant 3 | | |
| GPRIN1 | Homo sapiens G protein regulated | NM_052899 | SEQ ID NO: 310 |
| | inducer of neurite outgrowth 1 | | |
| C7orf68 | Homo sapiens chromosome 7 open | NM_013332 | SEQ ID NO: 311 |
| | reading frame 68, transcript variant 1 | | |
| HIGD1A | Homo sapiens HIG1 hypoxia | NM_001099669 | SEQ ID NO: 312 |
| | inducible domain family, member | | |
| | 1A, transcript variant 2 | | |
| HISPPD2A | Homo sapiens histidine acid | NM_001130859 | SEQ ID NO: 313 |
| | phosphatase domain containing 2A, | | |
| | transcript variant 6 | | |
| HMP19 | Homo sapiens HMP19 protein | NM_015980 | SEQ ID NO: 314 |
| HTT | Homo sapiens huntingtin | NM_002111 | SEQ ID NO: 315 |
| N/A | :c106175001-106173475 Homo | NC_000014 | SEQ ID NO: 316 |
| | sapiens chromosome 14, GRCh37 | | |
| | primary reference assembly IGHA1 | | |
| N/A | :c106209407-106207704 Homo | NC_000014 | SEQ ID NO: 317 |
| | sapiens chromosome 14, GRCh37 | | |
| | primary reference assembly IGHG1 | | |
| N/A | :90192948-90193424 Homo sapiens | NC_000002 | SEQ ID NO: 318 |
| | chromosome 2, GRCh37 primary | | |
| | reference assembly IGKV1D-13 | | |
| N/A | :22380474-23265085 Homo sapiens | NC_000022 | SEQ ID NO: 319 |
| | chromosome 22, GRCh37 primary | | |
| | reference assembly IGL@ | | |
| N/A | :23247168-23247205 Homo sapiens | NC_000022 | SEQ ID NO: 320 |
| | chromosome 22, GRCh37 primary | | |
| | reference assembly | | |
| IGLL3 | Homo sapiens immunoglobulin | NM_001013618 | SEQ ID NO: 321 |
| | lambda-like polypeptide 3 | | |
| N/A | :22734288-22735716 Homo sapiens | NC_000022 | SEQ ID NO: 322 |
| | chromosome 22, GRCh37 primary | | |
| | reference assembly | | |
| IGSF9B | Homo sapiens immunoglobulin | NM_014987 | SEQ ID NO: 323 |
| | superfamily, member 9B | | |
| KCND3 | Homo sapiens potassium voltage- | NM_172198 | SEQ ID NO: 324 |
| | gated channel, Shal-related | | |
| | subfamily, member 3, transcript | | |
| | variant 2 | | |
| KIAA1661 | Homo sapiens mRNA for | AB051448 | SEQ ID NO: 325 |
| | KIAA1661 protein, partial cds | | |
| KIF5C | Homo sapiens kinesin family | NM_004522 | SEQ ID NO: 326 |
| | member 5C | | |
| KIRREL3 | Homo sapiens kin of IRRE like 3 | NM_032531 | SEQ ID NO: 327 |
| | (Drosophila) | | |
| KRT222P | Homo sapiens keratin 222 | NM_152349 | SEQ ID NO: 328 |
| | pseudogene | | |
| LHFPL4 | Homo sapiens lipoma HMGIC | NM_198560 | SEQ ID NO: 329 |
| | fusion partner-like 4 | | |
| LOC100130100 | PREDICTED: Homo sapiens similar | XM_001716615 | SEQ ID NO: 330 |
| | to hCG26659 | | |
| LRRN3 | Homo sapiens leucine rich repeat | NM_001099660 | SEQ ID NO: 331 |
| | neuronal 3, transcript variant 1 | | |
| MAGI2 | Homo sapiens membrane associated | NM_012301 | SEQ ID NO: 332 |
| | guanylate kinase, WW and PDZ | | |
| | domain containing 2 | | |
| MAGT1 | Homo sapiens magnesium | NM_032121 | SEQ ID NO: 333 |
| | transporter 1 | | |
| MCTP2 | Homo sapiens multiple C2 domains, | NM_018349 | SEQ ID NO: 334 |
| | transmembrane 2 | | |
| MDGA2 | Homo sapiens MAM domain | NM_001113498 | SEQ ID NO: 335 |
| | containing | | |
| | glycosylphosphatidylinositol anchor | | |
| | 2, transcript variant 1 | | |
| CLEC18C | Homo sapiens C-type lectin domain | NM_173619 | SEQ ID NO: 336 |
| | family 18, member C | | |
| NEFH | Homo sapiens neurofilament, heavy | NM_021076 | SEQ ID NO: 337 |
| | polypeptide | | |
| NFASC | Homo sapiens neurofascin homolog | NM_015090 | SEQ ID NO: 41 |
| | (chicken) | | |
| NOS1AP | Homo sapiens nitric oxide synthase | NM_014697 | SEQ ID NO: 338 |
| | 1 (neuronal) adaptor protein, | | |
| | transcript variant 1 | | |
| NRXN1 | Homo sapiens neurexin 1, transcript | NM_004801 | SEQ ID NO: 339 |
| | variant alpha1 | | |
| NUP62 | Homo sapiens nucleoporin 62 kDa, | NM_153718 | SEQ ID NO: 340 |
| | transcript variant 3 | | |
| HAUS8 | Homo sapiens HAUS augmin-like | NM_033417 | SEQ ID NO: 341 |
| | complex, subunit 8, transcript | | |
| | variant 1 | | |
| OBFC2A | Homo sapiens | NR_024415 | SEQ ID NO: 342 |
| | oligonucleotide/oligosaccharide- | | |
| | binding fold containing 2A, | | |
| | transcript variant 2, transcribed | | |
| | RNA | | |
| PCDHA10 | Homo sapiens protocadherin alpha | NM_018901 | SEQ ID NO: 343 |
| | 10, transcript variant 1 | | |
| PCDHA6 | Homo sapiens protocadherin alpha | NM_018909 | SEQ ID NO: 90 |
| | 6, transcript variant 1 | | |
| PDPN | Homo sapiens podoplanin, transcript | NM_006474 | SEQ ID NO: 344 |
| | variant 1 | | |
| PGBD3 | Homo sapiens piggyBac | NM_170753 | SEQ ID NO: 345 |
| | transposable element derived 3 | | |
| P4HTM | Homo sapiens prolyl 4-hydroxylase, | NM_177938 | SEQ ID NO: 346 |
| | transmembrane (endoplasmic | | |
| | reticulum), transcript variant 3 | | |
| PHF21B | Homo sapiens PHD finger protein | NM_001135862 | SEQ ID NO: 347 |
| | 21B, transcript variant 2 | | |
| PMS2L5 | Homo sapiens postmeiotic | NM_174930 | SEQ ID NO: 348 |
| | segregation increased 2-like 5 | | |
| PRICKLE3 | Homo sapiens prickle homolog 3 | NM_006150 | SEQ ID NO: 349 |
| | (Drosophila) | | |
| PRUNE2 | Homo sapiens prune homolog 2 | NM_015225 | SEQ ID NO: 350 |
| | (Drosophila) | | |
| RAB1A | Homo sapiens RAB1A, member | NM_015543 | SEQ ID NO: 351 |
| | RAS oncogene family, transcript | | |
| | variant 2 | | |
| RANBP17 | Homo sapiens RAN binding protein | NM_022897 | SEQ ID NO: 352 |
| | 17 | | |
| RELL2 | Homo sapiens RELT-like 2, | NM_173828 | SEQ ID NO: 46 |
| | transcript variant 1 | | |
| RHBDD2 | Homo sapiens rhomboid domain | NM_001040457 | SEQ ID NO: 353 |
| | containing 2, transcript variant 2 | | |
| RMST | Homo sapiens rhabdomyosarcoma 2 | NR_024037 | SEQ ID NO: 354 |
| | associated transcript (non-protein | | |
| | coding), non-coding RNA | | |
| FLJ30058 | Homo sapiens hypothetical protein | NM_144967 | SEQ ID NO: 355 |
| | FLJ30058 | | |
| RPL10A | Homo sapiens ribosomal protein | NM_007104 | SEQ ID NO: 356 |
| | L10a | | |
| RPS24 | Homo sapiens ribosomal protein | NM_001142285 | SEQ ID NO: 357 |
| | S24, transcript variant d | | |
| RUNDC3A | Homo sapiens RUN domain | NM_001144825 | SEQ ID NO: 16 |
| | containing 3A, transcript variant 1 | | |
| SCAMP5 | Homo sapiens secretory carrier | NM_138967 | SEQ ID NO: 17 |
| | membrane protein 5 | | |
| SCG5 | Homo sapiens secretogranin V (7B2 | NM_001144757 | SEQ ID NO: 358 |
| | protein), transcript variant 1 | | |
| SERGEF | Homo sapiens secretion regulating | NM_012139 | SEQ ID NO: 359 |
| | guanine nucleotide exchange factor | | |
| SFT2D1 | Homo sapiens SFT2 domain | NM_145169 | SEQ ID NO: 360 |
| | containing 1 | | |
| SGMS2 | Homo sapiens sphingomyelin | NM_001136258 | SEQ ID NO: 361 |
| | synthase 2, transcript variant 3 | | |
| SLC22A4 | Homo sapiens solute carrier family | NM_003059 | SEQ ID NO: 362 |
| | 22 (organic cation/ergothioneine | | |
| | transporter), member 4 | | |
| SNAP25 | Homo sapiens synaptosomal- | NM_003081 | SEQ ID NO: 19 |
| | associated protein, 25 kDa, transcript | | |
| | variant 1 | | |
| ST8SIA4 | Homo sapiens ST8 alpha-N-acetyl- | NM_005668 | SEQ ID NO: 363 |
| | neuraminide alpha-2,8- | | |
| | sialyltransferase 4, transcript variant 1 | | |
| STXBP1 | Homo sapiens syntaxin binding | NM_003165 | SEQ ID NO: 364 |
| | protein 1, transcript variant 1 | | |
| SYNC | Homo sapiens syncoilin, | NM_030786 | SEQ ID NO: 365 |
| | intermediate filament protein, | | |
| | transcript variant 1 | | |
| SYPL1 | Homo sapiens synaptophysin-like 1, | NM_006754 | SEQ ID NO: 366 |
| | transcript variant 1 | | |
| TC2N | Homo sapiens tandem C2 domains, | NM_001128596 | SEQ ID NO: 367 |
| | nuclear, transcript variant 3 | | |
| TMEM145 | Homo sapiens transmembrane | NM_173633 | SEQ ID NO: 368 |
| | protein 145 | | |
| TMEM181 | Homo sapiens transmembrane | NM_020823 | SEQ ID NO: 369 |
| | protein 181 | | |
| TMEM198 | Homo sapiens transmembrane | NM_001005209 | SEQ ID NO: 370 |
| | protein 198 | | |
| TMEM25 | Homo sapiens transmembrane | NM_032780 | SEQ ID NO: 371 |
| | protein 25, transcript variant 1 | | |
| TMEM87A | Homo sapiens transmembrane | NM_015497 | SEQ ID NO: 372 |
| | protein 87A, transcript variant 1 | | |
| UBD | Homo sapiens ubiquitin D | NM_006398 | SEQ ID NO: 373 |
| VAMP2 | Homo sapiens vesicle-associated | NM_014232 | SEQ ID NO: 374 |
| | membrane protein 2 (synaptobrevin | | |
| | 2) | | |
| WIPI1 | Homo sapiens WD repeat domain, | NM_017983 | SEQ ID NO: 375 |
| | phosphoinositide interacting 1 | | |
| LOC91316 | Homo sapiens glucuronidase, beta/ | NR_024448 | SEQ ID NO: 376 |
| | immunoglobulin lambda-like | | |
| | polypeptide 1 pseudogene, non- | | |
| | coding RNA | | |
| APH1B | Homo sapiens anterior pharynx | NM_031301 | SEQ ID NO: 377 |
| | defective 1 homolog B (Celegans), | | |
| | transcript variant 1 (LOC145842) | | |
| FLJ37752 | Homo sapiens cDNA FLJ37752 fis, | AK095071 | SEQ ID NO: 378 |
| | clone BRHIP2023309 | | |
| LOC387895 | PREDICTED: Homo sapiens | XM_373553 | SEQ ID NO: 379 |
| | hypothetical gene supported by | | |
| | BC040060 | | |
| LOC730125 | PREDICTED: Homo sapiens | XM_001134301 | SEQ ID NO: 380 |
| | hypothetical LOC730125 | | |
| LOC652493 | PREDICTED: Homo sapiens similar | XM_001724425 | SEQ ID NO: 381 |
| | to pre-B lymphocyte gene 1 | | |
| FBLL1 | Homo sapiens fibrillarin-like 1, non- | NR_024356 | SEQ ID NO: 382 |
| | coding RNA | | |

*Transcript accession numbers beginning “ENST” are from the Ensembl Project database; all other accession numbers are from GenBank.

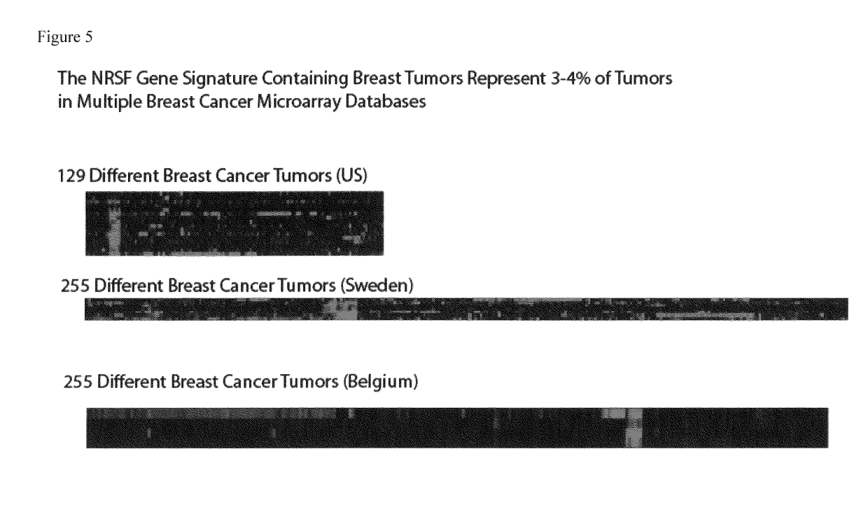

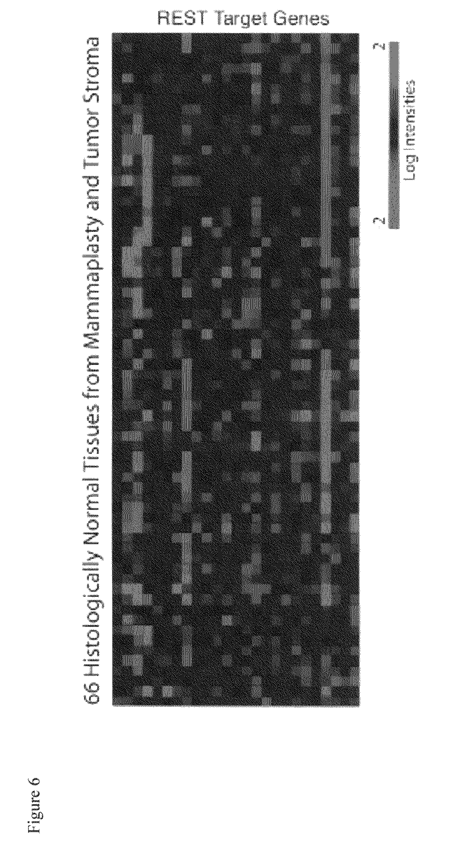

To verify the accuracy of these gene signatures and to determine whether loss of REST/NRSF function occurred exclusively in neoplastic mammary tissue, the 24-gene signature was used to screen 66 non-neoplastic mammary samples, half of which came from non-tumor bearing normal breast and half of which were adjacent normal stroma from a tumor-bearing breast (Finak et al., 2006, Breast Cancer Res 8:R58). The results of these assays are shown in FIG. 6. No cells exhibiting the RESTless phenotype were observed in any of the 66 stromal samples, suggesting that only carcinoma cells carry this defect in tumors.

### Example 3

### Tumors Positive for Gene Signature Express REST4 Truncated Variant

To determine the basis of REST/NRSF dysfunction in breast cancer, breast cancer cell lines were examined for REST/NRSF gene mutations and splice variants.

Tumor samples (including those that did and those that did not express a gene signature of the invention) were examined for the presence of either a REST/NRSF gene point mutation in the coding region or potential alternative splicing variants, specifically a REST4 truncated variant, a variant known in the art to be expressed in tumors but not in breast cancer. These experiments were performed as follows. RNA extracted from patient tumor samples was subjected to RT-PCR analysis. RNA was extracted from tumor biopsies obtained from patients using standard molecular biological techniques. Briefly, RNA was extracted using TRIzol (Invitrogen, Carlsbad, Calif.) and quantified using a Nanodrop product (Thermo Scientific, Wilmington, Del.). RNA (50 ng) was subjected to amplification using the Megascript kit (Ambion/Applied Biosystems, Austin, Tex.) to yield between 2-5 ug RNA. A portion of this amplified RNA (500 ng) was reverse transcribed into cDNA, and 5 ng cDNA used in subsequent PCR reactions.

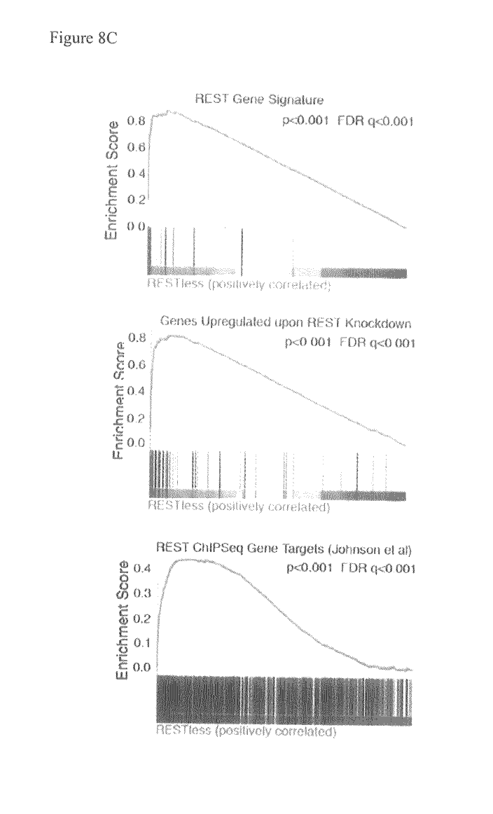

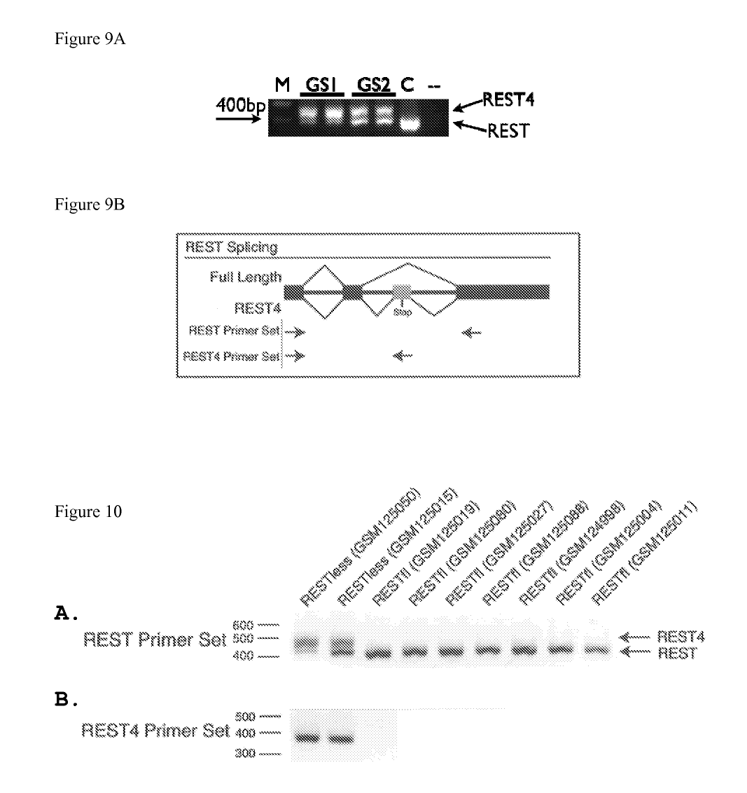

These assays showed that breast cancer tumor samples expressing a gene signature of the invention also expressed the REST4 splice variant, whereas tumors that did not express such a gene signature expressed full-length REST/NRSF (FIG. 9A). These data indicated that alternative splicing of REST/NRSF occurs in 4% of breast tumors and results in loss of REST/NRSF function and derepression of REST/NRSF target genes. In summary, these results indicated that REST/NRSF function is lost by alternative splicing in 4% of breast cancer tumors and is associated with expression of the gene signatures disclosed herein.

The primers utilized in the RT-PCR analysis shown in FIGS. 9A and 9B accurately identified REST4 variants, but other primer combinations and quantification/imaging strategies also can be utilized and are within the scope of this invention. Specifically, primers that flank the alternative exon that result in REST4 expression can be selected (labeled ‘N’ in Palm et al., 1999, Brain Res Mol Brain Res 72: 30). The sense primer can be in the first coding exon and anti-sense primer in the third coding exon. Amplification with these primers results in a 400 bp band for REST/NRSF and 450 bp for REST4. Alternatively, the sense primer can be located in the second coding exon or specific primers can be designed to identify a portion of the REST 4 exon sequence. However, other primer combinations are within the scope of this invention.

The two differential PCR products were reliably resolved on an agarose gel (as shown in FIGS. 9A and 9B). Alternatively the RT-PCR products can be distinguished by alternative means such as, for example, Real Time PCR incorporating CYBR green fluorescence. The basis for differentiation would be based on a higher melting point for the REST4 product (due to its larger size), which will manifest as a right-shifted melt-curve. Hence, two read temperatures (one below the 400 bp melt temperature and one between the 400 and 450 bp melt temperature) yield the total amount of REST/NRSF transcripts (REST+REST4) and also REST4 alone: the lower read temperature yielded REST+REST4 levels and the higher read temperature yielded REST4. The advantage of this approach was that both R4+and R4- tumors give a positive signal and provide a positive control for the assay. Thus a negative status call for REST4 would not be due to failure of any portion of the extraction/amplification protocol. Alternatively, an exon-N specific primer and primer in the neighboring exon can be used to generate a PCR product when REST4 is expressed. This can be quantified and compared to the signal from any number of housekeeping genes.

Testing RNA extracted from needle biopsies for REST4 status provided an alternative means for establishing NRST/REST functionality. Samples were examined for the presence of the gene signature. Tumors expressing a gene signature of the invention also showed increased levels of the REST/NRSF splice variant REST4.

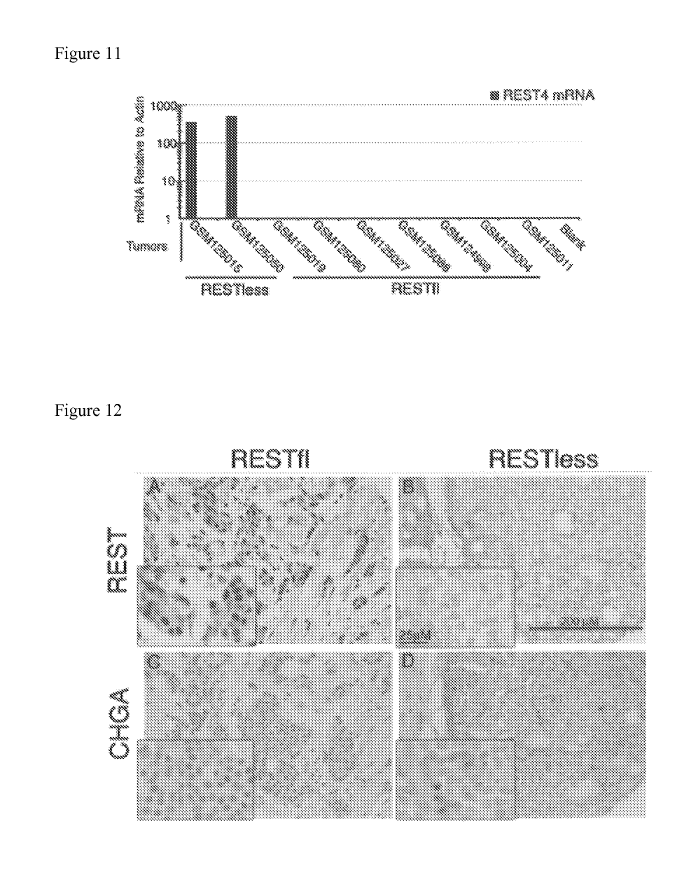

Whether aberrant REST/NRSF splicing could explain the loss of REST/NRSF function in breast cancer tumor samples was determined. RNA was extracted from two RESTless and seven RESTfl breast cancer tumor samples and amplified across REST/NRSF mRNA exon junctions using primers flanking the alternative REST4 exon (FIG. 10A). This analysis detected high levels of alternative splicing to produce REST4 in RESTless tumors, which could not be detected in RESTfl tumors (FIG. 10B). Selective amplification of REST4 using a primer placed in the REST4 exon confirmed the presence of the splice variant expression exclusively in the RESTless tumors (FIGS. 10B and 11).

The positive statistical correlation found as set forth above between expression of the gene signatures of this invention and lower disease-free survival times in breast cancer samples was confirmed for the correlation between poor disease-free survival and RESTless status (p=0.007), with the average time to relapse for RESTless tumors (14 months) being less than half the average for RESTfl tumors (35.9 months) (p=0.0217). RESTless tumors from this cohort also had significantly increased tumor size and lymph node involvement, alongside several other markers of aggressive, treatment-resistant breast cancers summarized in Table 5.

TABLE 5
Characteristics of RESTless Breast Cancer: Immunohistochemical analysis of REST/NRSF
staining in 182 breast tumors with corresponding patient outcome data.

| | | | | | | Average | | | |
|---|---|---|---|---|---|---|---|---|---|
| | | Chromo- | | | | Time to Total | | | |
| | | granin | HER2 | | | Relapse | | Nodal | Percent |
| | | Pos. | Pos. | Grade | Size | (months) | Age | Number | Relapse |
| All | RESTless | 10.8% | 13.5% | 2.41 +/− 0.13 | 3.88 +/− 0.39 | 14.0 +/− 1.8 | 53.4 +/− 2.19 | 4.8 +/− 1.2 | 43% |
| Tumors | (n = 37) | (0.0007) | (0.496) | (0.0269) | (0.0012) | (0.0217) | (0.0494) | (0.020) | (0.054) |
| | RESTfl | 0.7% | 9.7% | 2.07 +/− 0.07 | 2.65 +/− 0.16 | 35.9 +/− 3.02 | 58.3 +/− 1.11 | 2.6 +/− 0.4 | 27.0% |
| | (n = 145) | | | | | | | | |
| ER+ | RESTless | 15% | 10% | 1.95 +/− 0.18 | 3.49 +/− 0.55 | 17.3 +/− 2.6 | 55.45 +/− 3.3 | 3.95 +/− 1 | 35% |
| Tumors | (n = 20) | (0.0007) | (0.0164) | (0.45) | (0.0142) | (0.127) | (0.0716) | (0.127) | (0.161) |
| | RESTfl | 0.9% | 2.6% | 1.86 +/− 0.07 | 2.59 +/− 0.17 | 42.4 +/− 3.82 | 60.00 +/− 1.2 | 2.3 +/− 0.4 | 23.0% |
| | (n = 115) | | | | | | | | |
| Triple Neg | RESTless | 7.7% | | 2.92 +/− 0.1 | 3.45 +/− 0.3 | 5 +/− 0.88 | 50.8 +/− 3.5 | 6.92 +/− 2.4 | 46% |
| Tumors | (n = 13) | (0.233) | | (0.217) | (0.0568) | (0.0109) | (0.0895) | (0.168) | (0.26) |
| | RESTfl | 0.0% | | 3.00 +/− 0 | 2.47 +/− 0.4 | 16.6 +/− 1.9 | 53.0 +/− 2.8 | 3.47 +/− 1.1 | 26.0% |
| | (n = 19) | | | | | | | | |

Figures shown represent the mean value plus or minus the standard error of all samples in the indicated cohort, with the Pearson chi-squared test for independence with the indicated p-value. Bold values indicate parameters with statistically significant (p < 0.05) correlation with RESTless tumors.

In addition, patients with so-called “triple negative (TN) tumors” (i.e., Estrogen Receptor (ER)⁻/Progesterone Receptor⁻/HER2⁻) that were also RESTless endured significantly greater disease recurrence within 2 years than TN/RESTfl patients (50% versus 20% recurrence (p=0.044, n=32)). Patients with RESTless ER+ breast tumors were also more prone to relapse in the first 3 years (p=0.003, n=135). Strikingly, 100% of disease recurrence events for patients with RESTless tumors occurred in the first 36 months, compared to 61% of recurrence events for patients with RESTfl tumors. Importantly, after 3 years, there were no additional recurrences of RESTless tumors. These data indicate that the presence of REST4 leads to a more aggressive disease, which is more likely to recur within 3 years of diagnosis.

These results demonstrated that REST/NRSF function is lost in a fraction of breast tumors. The loss of REST/NRSF function was due in these tumors to alternative splicing of REST/NRSF, and RESTless tumors were associated with aggressive, rapid recurrence and poor prognosis.

### Example 4

### Immunohistochemical Analysis of REST/NRSF Truncated Protein in Breast Cancer

To determine the frequency of REST/NRSF protein truncation in breast cancer, an immunohistochemical (IHC) screen was developed using an antibody directed to the C-terminus of REST/NRSF (Atlas Antibodies, Stockholm). REST4 and a truncated form of REST/NRSF identified as a SNP in colon cancer (Westbrook et al., 2005, Cell, 121:837-848) are not recognized by this antibody, permitting all tumors lacking full-length REST to be identified specifically. RESTless tumors lacked antibody staining, whereas RESTfl exhibit nuclear staining

REST labeling was performed using a Lab Vision Autostainer 360 (Thermo Fischer Scientific Fremont, Calif.) as follows. After deparaffinization, heat-induced epitope retrieval with citrate buffer and endogenous peroxidase inhibition was performed, and the slides then blocked with Background Sniper™ (Biocare Medical, Concord, Calif.). The sections were then incubated with rabbit anti-REST antibody (HPA006079, Sigma-Aldrich St Louis, Mo.) at a concentration of 0.5 μg/mL for 60 minutes. After washing, Mach3™ detection system (Biocare Medical, Concord, Calif.) was applied. The labeling reaction was manually scored by a board-certified pathologist for cytoplasmic and nuclear carcinoma cell compartments, using the method described by Harvey and colleagues (Harvey et al., 1999, J Clin Oncol 17:1474-81).

Immunohistochemical analysis of 182 breast tumors in a tissue microarray confirmed the lack of full-length nuclear, and therefore functional REST/NRSF predicted by the REST4 splicing in 37 tumor samples (results shown in FIGS. 12 and 12B).

As an additional measure of REST function, breast cancer tissue sections were stained for ectopic expression of chromogranin A, a REST target gene and a component of the 24-gene REST gene signature. Chromogranin A is a secreted factor that is seldom found outside the nervous system /neuroendocrine tumors. Four-micron sections of previously characterized tissue microarrays, which contain duplicate tissue cores from 207 human breast carcinomas, were used for labeling experiments (Baba et al., 2006, Breast Cancer Res Treat 98:91-8). Chromogranin A labeling was performed on an automated Ventana instrument (Ventana Medical Systems, Tucson, Ariz.). After standard deparaffinization, epitope retrieval was performed with CC1 high-pH buffer (Ventana Medical Systems). In the automated protocol provided by the instrument manufacturer, the prediluted anti-chromogranin A antibody (Clone LK2H10, Ventana Medical Systems) was added to the deparaffinzed tissue samples for 32 minutes at 42° C. A universal secondary antibody was then added, and target detection was accomplished with an indirect biotin-avidin-peroxidase procedure provided by the manufacturer.

In RESTless tumors, chromogranin A expression was found to be upregulated by several orders of magnitude above what is seen in normal breast. Interestingly, samples that stained negative for REST/NRSF showed a statistically significant enrichment in staining for the REST target chromogranin-A (CHGA), consistent with a loss of REST/NRSF repression (p<0.01; FIGS. 12C and 12D).

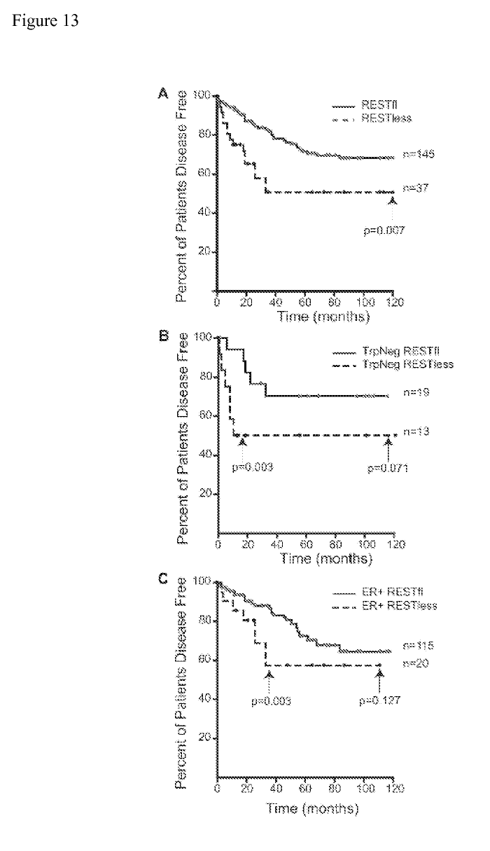

Lack of REST/NRSF function correlates with poor cancer prognosis. The absence of the C-terminal domain in REST4 mutants provided a means for IHC screening for loss of full-length REST/NRSF using an antibody raised against the C-terminus of REST. Immunohistochemical analysis on the panel of 182 tumor samples with associated outcome data showed that patients with RESTless tumors experience a 20% reduction in disease free survival over 10 years when compared to their RESTfl counterparts (p=0.007), as shown in FIG. 13. The majority of the outcome disparity between patients with RESTless and RESTfl tumors occurs in the first three years post-diagnosis. Fifty percent of patients with RESTless tumors showed recurrence within three years, which represented 100% of all patients with RESTless tumors that relapsed in this data set. By comparison, 16% of patients with RESTfl tumors showed recurrence within three years. RESTless tumors strongly correlated with decreased time to disease recurrence, increased tumor size, and a higher number of lymph node metastases, all of which demonstrated a more aggressive disease course (Table 5).

Remarkably, RESTless tumors were found in all histological classes of breast tumors, and all classes showed a poorer prognosis without functional REST. RESTless triple negative tumors showed a particularly aggressive disease course. Of the 32 triple negative tumors screened, 13 were found to be RESTless, six (46%) of which recurred in the first 12 months post-diagnosis, compared to just one of the 19 (5%) RESTfl triple negative tumors (p=0.003). However, no TN RESTless tumor recurred after 12 months in 10 years of patient outcome data. ER+ RESTless tumors showed a similar pattern of early recurrence, wherein eight of 21 (38%) patients saw disease recurrence in the first 36 months, compared to just 11% of ER+ RESTfl patients (p=0.003). Thereafter, none of the remaining 13 disease free patients with ER+ RESTless tumors experienced recurrence, compared to 12 of the 102 remaining disease-free ER+ RESTfl patients. These data suggest that RESTless tumors represent a distinct, aggressive subset of breast tumors with a unique disease course.

The above immunohistochemical analyses produced a robust screen that can be taken to the clinic to assess REST4 expression in breast tumors, which can facilitate early diagnosis of negative prognosis for around 10,000 breast cancer patients per year in the U.S.

### Example 5

### REST/NRSF Knockdown Increases Tumor Growth in Mice

To determine whether REST loss is a marker or driver of tumor aggression, xenograft experiments were performed to measure the effect of REST knockdown on tumor growth in nude mice. The studies provided herein illustrated that REST is lost in 20% of breast cancers, and that these “RESTless” tumors are highly aggressive (Wagoner et al., 2010, PLoS Genet, 6: e1000979). These studies further demonstrated that REST is a direct transcriptional repressor of the tumor promoter LIN28. In vitro and in vivo data presented herein further showed that LIN28 expression was a critical factor for increased tumorigenicity of REST knockdown cells, and demonstrated that LIN28 mRNA levels were increased in human breast cancers lacking REST.

Control (shCon) or REST knockdown (shREST) MCF7 cells were injected subcutaneously into the flanks or mammary fat pads of female athymic nude mice, and tumor growth was measured. Adult intact female athymic nude-Foxn1^{nu} mice (Harlan Laboratories, Indianapolis, Ind.) were used for xenograft studies. MCF7 cells were suspended in a cold 1:3 Matrigel/DMEM solution, and 10⁶ cells were injected per injection site. Each mouse received two subcutaneous flank injections as well as subcutaneous injections into the fat pads of the 4^{th} and/or 9^{th} mammary glands. Tumors were monitored weekly by palpation and caliper measurements. Statistical analysis was done using Mstat software; Kaplan-Meier and Logrank survival analyses were performed on tumor take data, while tumor burden was evaluated using the Wilcoxon rank sum test, and two-sided p-values were used throughout.

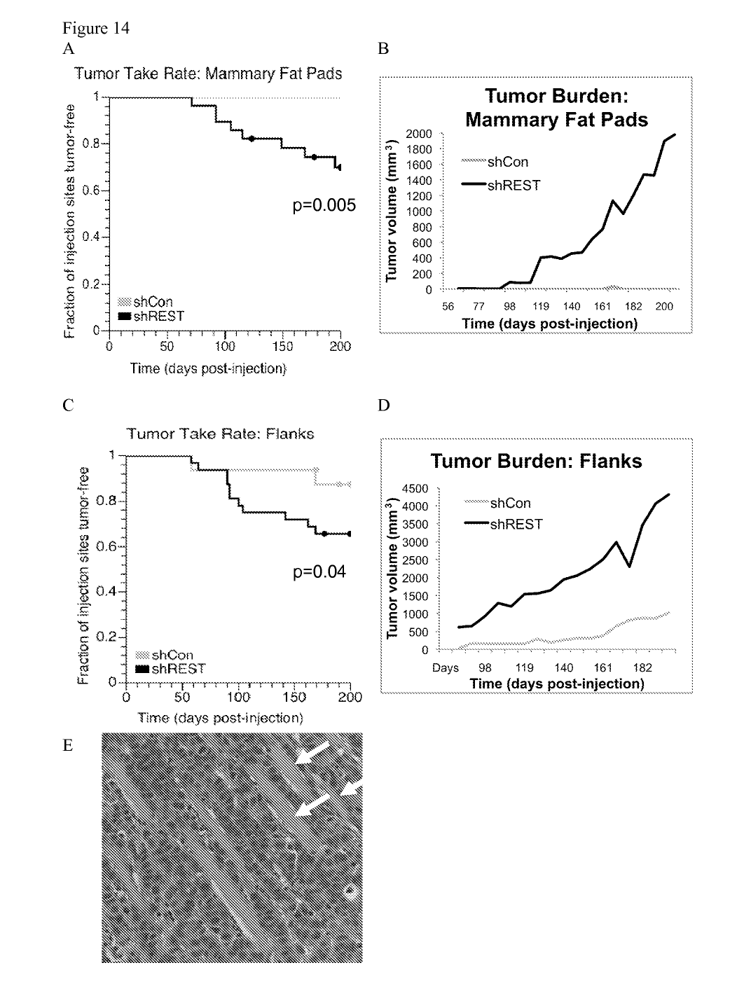

By 100 days post-injection, the tumor take rate was significantly greater for shREST than shCon tumors (p=0.018; at 200 days, p=0.0005). Tumor take rate and growth by injection site were further analyzed. Two hundred days post-injection, 25% (7/28) of shREST mammary fat pad injection sites had given rise to tumors, compared with 0% (0/28) of shCon injections (p=0.005, FIG. 14A). The total tumor burden for shREST mammary fat pad tumors was 1458 mm³, versus 0 mm³ for shCon tumors (p=0.005, FIG. 14B). The tumor take rate was also significantly increased for shREST versus shCon MCF7s when injected subcutaneously into the flanks of the nude mice, with 34.4% (11/32) of shREST injection sites giving rise to tumors, while only 12.5% (4/32) of shCon injections gave rise to tumors by 200 days post-injection (p=0.040, FIG. 14C). The total tumor burden was significantly greater for shREST than shCon tumors, at 3885 mm³ and 867 mm³, respectively (p=0.037, FIG. 14D).

In conclusion, the REST knockdown resulted in a statistically significant increase in tumorigenicity of MCF7 cells at both the orthotopic mammary fat pad and the flank injection sites. The shREST tumors were epithelial in phenotype, highly anaplastic, displayed a high mitotic rate and exhibited nuclei that varied greatly in size. In addition, 62.5% (5/8) of shREST flank tumors examined show localized invasion into adjacent muscle (FIG. 14E). These data illustrate that a loss of REST function causes an increase in cancer aggression.

### Example 6

### REST/NRSF Induces Expression of Tumor Promoter LIN 28

The results set forth herein, particularly in Example 4, established that REST/NRSF is lost in a distinct subset of breast tumors. Moreover, breast cancer tumors and cell lines that lack REST/NRSF functionality exhibited elevated LIN28 expression.

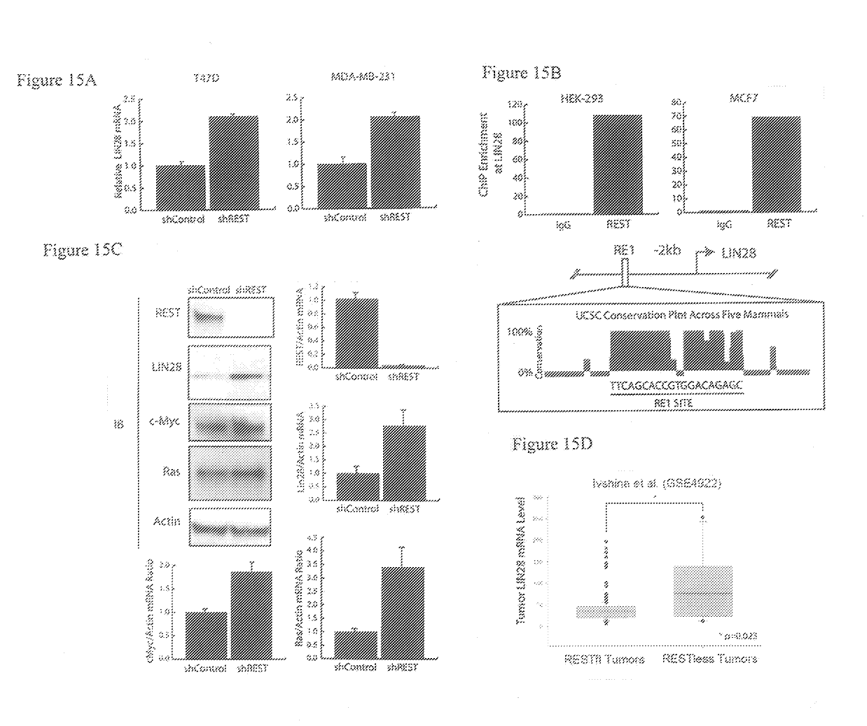

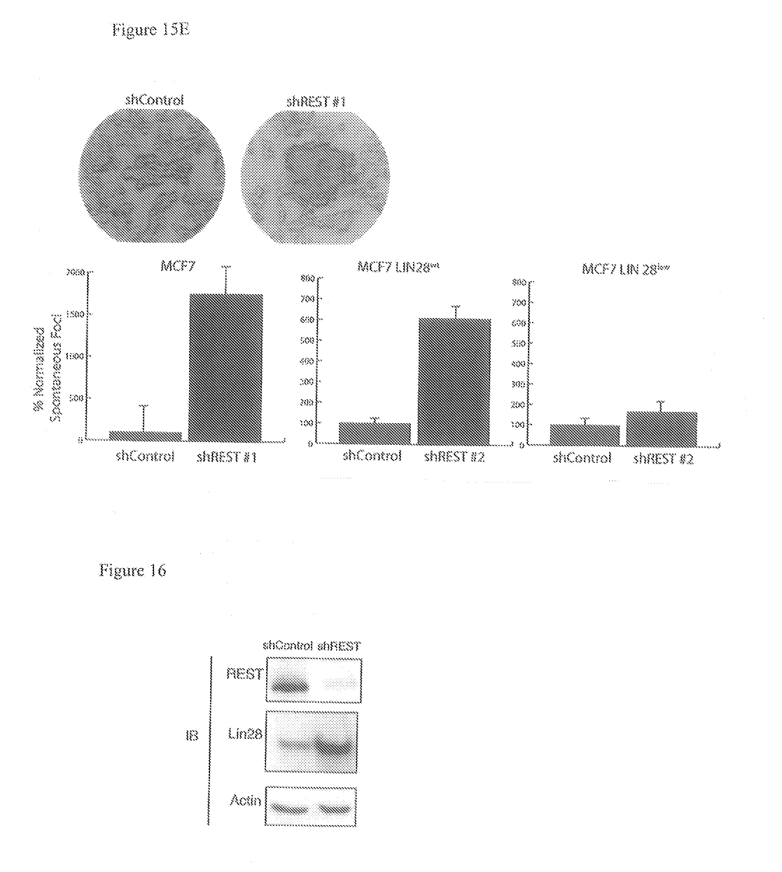

In an effort to understand the basis for poor clinical outcomes experienced by patients with RESTless breast cancer, DNA microarrays of REST/NRSF knockdown cell lines were probed for genes upregulated by a loss of REST/NRSF that have been linked to aggressive cancer. Expression of the tumor promoter and pluripotency factor LIN28 was found to be elevated in response to REST/NRSF knockdown in multiple cell lines including T47D and MDA-MB-23 (FIG. 15A).

LIN28 mRNA levels were assessed using real time reverse-transcriptase PCR (qRT-PCR). RNA was harvested from cells using Trizol (Invitrogen, Carlsbad, Calif.), and reverse transcribed using Superscript III reverse transcriptase (Invitrogen, Carlsbad, Calif.) per the manufacturer's instructions. cDNA was amplified using Takara SYBR Premix ExTaq on an MJR Opticon II real-time thermocycler with 20 ng of RNA equivalent cDNA per reaction. All qRT-PCR experiments were performed in triplicate comparing gene expression between cell lines using beta actin mRNA levels as a normalizing control. Chromatin immunoprecipitation experiments were performed as previously described (Roopra et al., 2004, Mol. Cell 14: 727-38, incorporated by reference herein) using Santa Cruz anti-REST antibody H-290. Chromatin immunoprecipitation (ChIP) data are presented as fold-enrichment of H-290 antibody over a non-targeting IgG antibody. Western blots were imaged and quantified on a Kodak Imagestation 2000R using Kodak 1D image analysis software (Carestream Health Rochester, N.Y.).

Sequence analysis showed that an RE1 sequence was present 2 kb upstream from the human LIN28 promoter. ChIP experiments using HEK-293 and MCF7 cells revealed that REST/NRSF binds the LIN28 RE1 (FIG. 15B), suggesting that the site is functional. Additionally, knockdown of REST/NRSF resulted in increased LIN28 mRNA (FIG. 15A) and protein (FIG. 15C and FIG. 16) in multiple cell lines. Together, these results demonstrated that LIN28 was a direct target of REST/NRSF repression.

Given the role of LIN28 in suppressing maturation of the let-7 family of microRNAs, it was expected (in view of the results disclosed herein) that the let-7 target genes c-Myc and Ras would be upregulated upon REST/NRSF knockdown. This analysis was performed and confirmed in MCF7 cells (FIG. 15C). In aggregate, the data illustrated that REST/NRSF dysfunction induced expression of LIN28 and at least two of its oncogenic target genes, c-Myc and Ras.

LIN28 was found to be over-expressed in RESTless tumors. Analysis of cDNA microarray data from 289 breast tumors showed that the median expression level of LIN28 in RESTless tumors was greater than the 90^{th} percentile expression in RESTfl tumors (p<0.05) (FIG. 15D), further supporting the in vitro findings.

LIN28 has been shown to contribute to cellular transformation in other cell lines (Dangi-Garimella et. al., 2009, EMBO J28:347-58;Viswanathan, et. al., 2009, Nat Genet 41:843-48). Loss of REST/NRSF function also induced focus formation in a LIN28-dependent manner. MCF7 breast cancer cells formed spontaneous foci following REST/NRSF knockdown (FIG. 15E). This phenotype was used to determine whether LIN28 overexpression in RESTless breast cancer tumor cells conferred a growth advantage to breast tumor cells. In these experiments, MCF7 cells stably expressing shRNAs as described above were trypsinized (Cellgro 0.25% Trypsin MT 25-050-CI, Mediatech, Inc Manassas, Va.) for 2 min at room temperature and repeatedly aspirated. One million MCF7 cells were plated per 100 mm plate and allowed to grow for 72 hours, followed by methanol fixation. Plates were stained with Giemsa stain (Fluka Analytical catalog #11700, Sigma-Aldrich St Louis, Mo.) for 30 minutes. Stained plates were scanned and foci were quantified using NIH ImageJ Research Services Branch, National Institute of Mental Health, Bethesda, Md.).

Foci detected in this manner were trypsin-resistant aggregates of shREST-expressing MCF7 cells that readily formed in subconfluent cell culture. After typsinization and resuspension, foci sedimented rapidly, and continued to grow following passage. REST/NRSF knockdown using either of two anti-REST shRNAs gave rise to foci in sub-confluent cell culture, whereas the control infection with lentivirus expressing a non-targeting shRNA failed to generate foci (FIG. 15E). These focus formation assays were repeated using MCF7 cells stably expressing either anti-LIN28 shRNA (LIN28^{low}), which repressed LIN 28, or non-targeting control (LIN28^{WT}) shRNA, which was a negative control and did not impact LIN 28 levels. In the LIN28^{WT} background, shREST lentiviral particles induced a six-fold increase in focus formation over those re-treated with shCon lentiviral particles (FIG. 15E). However, in LIN28^{low} MCF7s, loss of REST/NRSF failed to induce focus formation.

Specific inhibition of LIN28 in cells deficient for REST/NRSF resulted in focus formation. Indeed, these studies showed that LIN28 knockdown was sufficient to inhibit the increased focus formation induced by REST/NRSF knockdown (FIG. 15E). It was found that RESTless breast tumors also have higher LIN28 mRNA expression levels, supporting a functional role for LIN28 in breast cancer tumors in vivo. As set forth herein, LIN28 was also shown to be upregulated in GSE4922 RESTless breast tumors. It should be noted however that LIN28 was not part of the RESTless 24-gene signature, because although LIN28 expression was induced upon REST/NRSF knockdown in T47D and HEK cells, it was not increased in MCF10a cells. Nonetheless, LIN28 provides a useful single-gene gene signature for the identification of RESTless tumors. Given the higher levels of lymph node metastasis in RESTless breast cancer and the aberrant expression of LIN28 in other aggressive cancers, the studies described herein support the role of LIN28 as a key contributor to the aggressive nature of RESTless breast cancer, and an important marker and gene signature for aggressive forms of breast cancer in vivo.

In summary, the results of the experiments set forth herein demonstrated that RESTless tumors represent a distinct, aggressive subset of breast tumors with a unique disease course. REST/NRSF status is an important predictor of poor prognosis that correlated with increased lymph node metastasis and early disease recurrence. REST/NRSF is an important regulator of LIN28, a protein involved in tumorigenesis in several cancer types. In view of LIN28's role in focus formation and other attributes of aggressive cancers, LIN28 overexpression in RESTless breast tumors is an important gene signature for aggressive breast cancers.

### Example 7

### Tumor Promoter LIN28 is a Direct Target of Transcriptional Repression by REST/NRSF

As described in Example 1, knockdown REST cells were produced in HEK-293, T47D and MCF10a cell lines. MCF7, normal murine mammary gland (NMuMG) and HEK-293 cells were grown in DMEM and T47Ds in RPMI, all supplemented with 10% fetal bovine serum, 100 IU/ml penicillin, 100 μg/ml streptomycin and 250 ng/ml amphotericin-B. NMuMG and T47D cells were additionally supplemented with 10 μg/ml insulin. All cells were grown at 37° C. in 5% CO₂. Stable REST knockdown was achieved using a Dharmacon SMARTvector lentiviral shRNA delivery system, as per manufacturer's instructions (also described in Wagoner et al., 2010, PLoS Genet, 6: e1000979). Stable knockdown of LIN28 (shLIN28) was achieved by infecting cells with lentivirus expressing an anti-LIN28 shRNA (clone TRCN0000102579) in a pLKO.1 vector obtained from Open Biosytems (Huntsville, Ala.). Lentiviral particles were generated and MCF7 cells infected according to Addgene's pLKO.1 protocol (www.addgene.org/pgvec1?f=c&cmd=showcol&colid=170&page=2; incorporated by reference herein).

Upon REST knockdown, the tumor promoter and master regulator of microRNA processing LIN28 is upregulated in T47D and HEK-293 cells. Because LIN28′s potential upregulation is associated with a variety of advanced cancers (Viswanathan, et al., 2009, Nat Genet, 41:843-48), and because of LIN28′s potential role in breast cancer aggression and metastasis (Dangi-Garimella et al., 2009, EMBO J, 28:347-358), the regulatory relationship between REST and LIN28 and the role of LIN28 in RESTless aggression was further characterized.

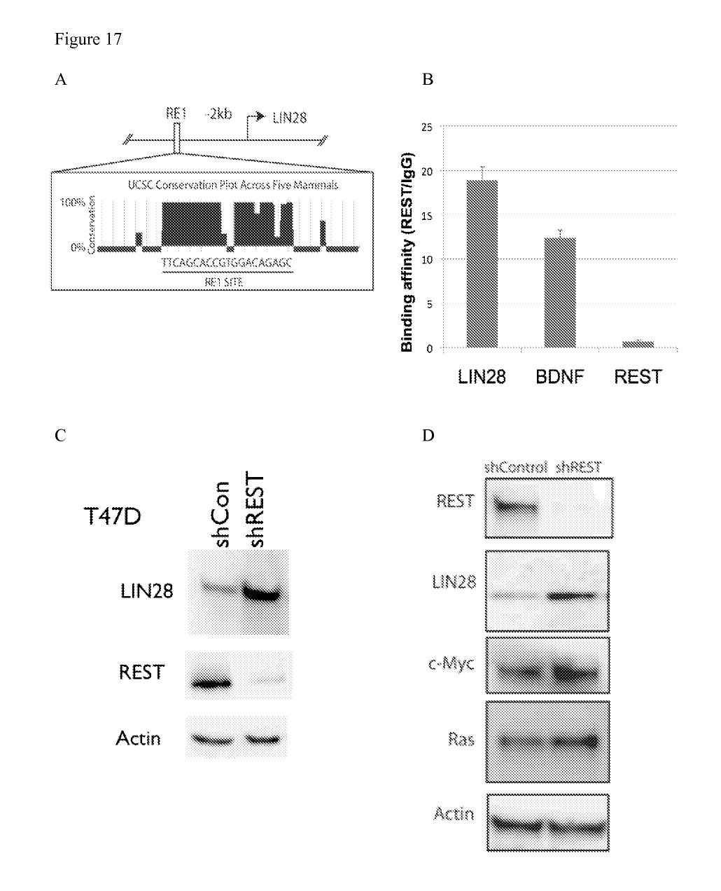

The following studies were performed to determine if an increase in LIN28 expression observed upon REST knockdown was a direct result of REST loss. Sequence analysis showed that the LIN28 promoter contains a REST binding site (RE1) ˜2 kb upstream of the transcriptional start site, and conservation analysis demonstrates that this RE1 site is evolutionarily conserved among mammals (a diagrammatic representation of this conservation is shown in FIG. 17A). Quantitative chromatin immunoprecipitation revealed that REST binds this RE1 site with high affinity.

In the performance of chromatin immunoprecipitation studies, cells were fixed with formaldehyde (1%) at 37° C. for 10-15 minutes, washed with cold PBS and harvested into lysis buffer (150 mM NaCl, 10% glycerol, 0.3% Triton X-100, 50 mM Tris pH 8.0, protease inhibitor) followed by sonication on ice and centrifugation at 12,000×g for 30 min. 2 μg of anti-REST antibody (H-290, Santa Cruz Biotech, Santa Cruz, Calif.) or rabbit IgG (Sigma-Aldrich, St. Louis, Mo.) was added 300 μg total protein and agitated overnight at 4° C. Samples are centrifuged at 12,000×g for 30 min and supernatant was incubated with protein G Sepharose beads (previously blocked with herring sperm DNA and BSA) for 1 hour at 4° C. with agitation. Supernatant was removed and beads were rinsed once and then washed four times for 5 minutes on ice with wash buffer (500 mM NaCl, 0.1% SDS, 1% Triton X-100, 2 mM EDTA, 20 mM Tris pH 8.1). Wash buffer was removed and beads were incubated overnight at 64° C. in 0.2M NaCl, 1% SDS, 0.1% NaHCO₃. DNA was isolated by phenol-chloroform extraction and isopropanol precipitation and analyzed by quantitative real-time PCR as previously described using the following primers:

Human LIN28:
(AGC GGG AAC CGG CAT TGA GGA A [SEQ ID NO: 383];
AAA GGG GAG TTG AAC GCT CTG GCT TCT [SEQ ID NO:
384]).
Human BDNF:
(TTACAGCGCGGCCAAGAAGACTAC [SEQ ID NO: 385];
CCA TCC GCA CGT GAC AAA CC [SEQ ID NO: 386]).
Human REST:
(TGG CCG CAC CTC AGC TTA TTA TG [SEQ ID NO: 387];
AGG CTG AGG TTC TAC GAC GCT GAG [SEQ ID NO:
388]).
Mouse BDNF:
(TCG CAT ACG TGG AAA GGG TCT CAT [SEQ ID NO:
389];
CAA ATC CGC TGG CTC TGT CC [SEQ ID NO: 390]).
Mouse LIN28:
(ATG TGT GTC AGG AGA CTT CGG AGG [SEQ ID NO:
391];
ATC ACT TGC TCT GTC CAG GGT G [SEQ ID NO: 392]).

Lysates from MCF7 cells were immunoprecipitated with an anti-REST or anti-IgG (sham) antibody, and their association with the LIN28, BDNF (positive control) and REST (negative control) promoter regions was assessed. The affinity of REST for each promoter region was calculated as the -fold increase in DNA precipitated with anti-REST versus sham IgG antibody. In these experiments, REST bound the LIN28 RE1 site with high affinity, approximately twice as tightly as it bound to the RE1 of BDNF, a canonical REST target gene (19-fold and 12-fold, respectively, FIG. 17B). As expected, REST din not bind to its own promoter region, which does not contain an RE1 site, with greater specificity than does IgG. Upon REST knockdown, REST binding at both the LIN28 and BDNF RE1 sites was ablated (data not shown). The high affinity of REST for the LIN28 promoter and its loss from the promoter upon REST knockdown was also observed in HEK and normal murine mammary gland (NMuMG) cells.

To determine whether REST binding to the LIN28 RE1 site correlated with LIN28 repression, LIN28 protein levels in control (shCon) and REST knockdown (shREST) MCF7 and T47D cells were measured by immunoblotting experiments. For immunoblotting, cells were washed with cold PBS and harvested into lysis buffer (150 mM NaCl, 10% glycerol, 0.3% Triton X-100, 50 mM Tris pH 8.0) followed by sonication on ice and centrifugation at 12,000×g for 30 min. Proteins were resolved via SDS-PAGE and transferred to PVDF. Immunoblotting was performed with antibodies raised against and immunospecific for REST (Upstate 05-579), LIN28 (Cell Signaling Technologies #3978, Danvers, Mass.), and beta-actin (MP Biomedicals, Solon, Ohio) and visualized with enhanced chemiluminescence (Thermo Fisher, Rockford, Ill.).

The results show that when REST was knocked down and lost from the LIN28 RE1 site, LIN28 expression increased in both cell lines (as shown in FIGS. 17C and 17D). Given the role of LIN28 in suppressing maturation of let-7 family miRNAs, it was expected that the let-7 target genes c-Myc and Ras would be upregulated upon REST knockdown, and this was confirmed in MCF7 cells (FIG. 17D). These results established that REST was a direct transcriptional repressor of LIN28, and that loss of REST was sufficient to induce aberrant expression of LIN28 and two of its oncogenic target genes, c-Myc and Ras.

REST knockdown also increased migration in MCF7 cells in a LIN28-dependent manner. The migratory capacity of shCon and shREST MCF7s were examined by a modified Boyden chamber assay. Serum-starved MCF7s were allowed to migrate for 24 hours across a filter with 8 μm pores towards 10% FBS. MCF7 cells were serum-starved (0% FBS) overnight, and then 5×10⁴ cells were seeded into a modified Boyden chamber and allowed to migrate across a filter (8 um pore size) towards media containing 10% FBS for 24 hours. Cells that did not migrate were removed with a cotton swab and filters fixed in methanol at −20° C. prior to staining with Hoechst 33258 (0.5 μg/ml, Sigma Aldrich, St. Louis, Mo.). Nuclei of migrated cells were photographed at 20× magnification and counted using NIH ImageJ.

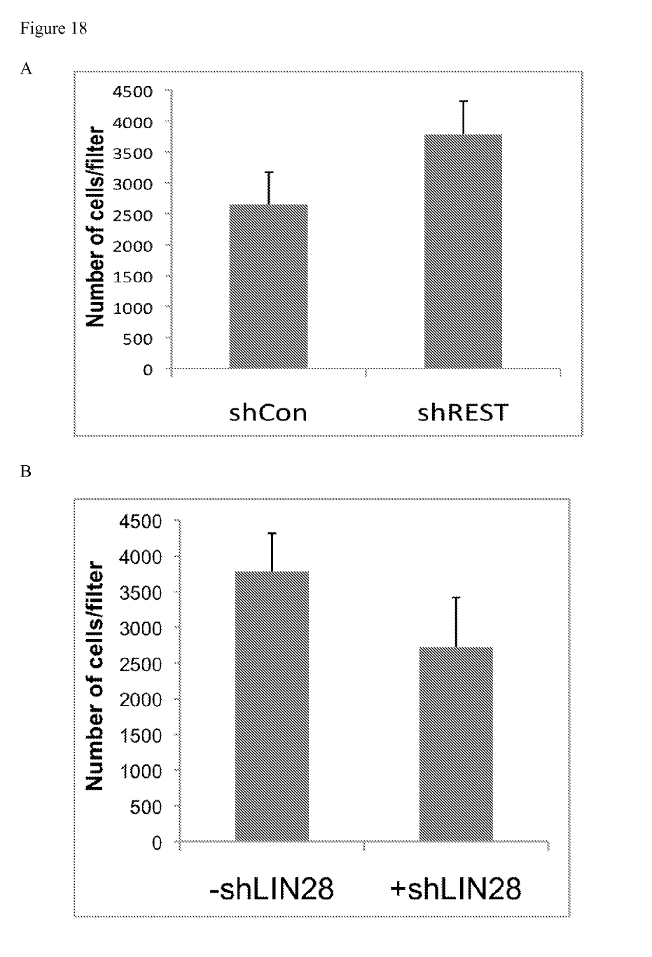

shREST cells showed an increased migratory capacity relative to shCon cells (FIG. 18A, p=0.025). To evaluate the contribution of LIN28 to migration in shREST MCF7s, cells were further infected with a lentiviral construct expressing an anti-LIN28 (+shLIN28) or control (−shLIN28) shRNA and the migration assay was repeated. It was found that knockdown of LIN28 in the shREST background reduced the migratory capacity of these cells (p currently =0.046).

### Example 8

### Increased LIN 28 Expression Contributes to RESTless Tumor Formation and Increased LIN 28 Expression is Observed in RESTless Breast Tumors

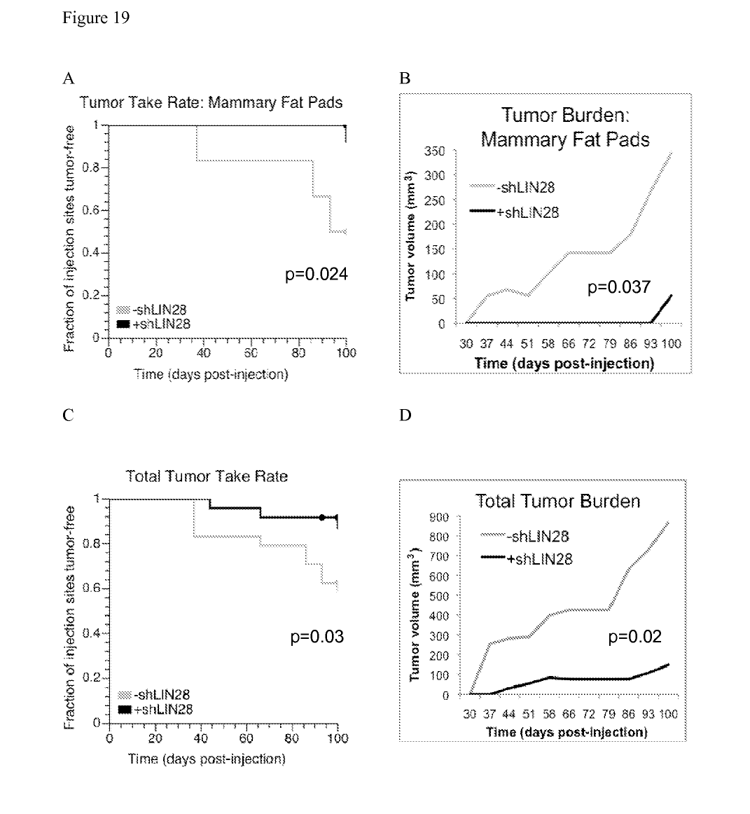

To determine whether upregulation of LIN28 observed in shREST cells contributed to tumorigenicity of RESTless cells in vivo, tumorigenicity of shREST cells with and without increased LIN28 expression was compared. shREST MCF7 cells expressing a control (−shLIN28) or anti-LIN28 shRNA (+shLIN28) were injected subcutaneously into the flanks and mammary fat pads of athymic nude mice as described above. After 100 days, 50% (6/12) of control mammary fat pad injections had given rise to tumors, compared with only 8.3% (1/12) of fat pads injected with LIN28 knockdown cells (p=0.024, results shown in FIG. 19A). The tumor burden in the mammary fat pads was also significantly decreased when LIN28 was knocked down, with a total tumor volume of 345mm³ for control compared to only 56mm³ for LIN28 knockdown tumors (p=0.037, FIG. 19B).

Overall, by 100 days post-injection, 42% (10/24) of control injections had given rise to measurable tumors (>3 mm in diameter), versus 12.5% (3/24) of LIN28 knockdown injections (FIG. 19C, p=0.03). The tumor burden was also significantly larger for tumors expressing LIN28 relative to their +shLIN counterparts (p=0.02, FIG. 19D). Thus, LIN28 expression is required for the enhanced tumorigenicity of shREST cells.

To determine whether these in vitro and in vivo findings regarding the contribution of LIN28 to RESTless MCF7 tumorigenicity had potential clinical relevance, LIN28 expression in tumors from human patients with RESTless breast cancer was assessed. As previously described in Wagoner et al., 2010, PLoS Genet, 6: e1000979, bioinformatic analyses on the microarray data were performed using BRB-ArrayTools v3.7 (developed by Dr. Richard Simon and BRB-ArrayTools Development Team) and MultiExperiment Viewer 4.5.1. Tumor gene expression data were obtained from the NCBI Gene Expression Omnibus, and identified by their GEO dataset record number. Analysis of dataset GSE6532 was performed to determine the aggressiveness of tumors identified as being RESTless using the gene signature method. All samples from this dataset that included information on duration of relapse-free survival as well as relapse event information were included in this analysis.

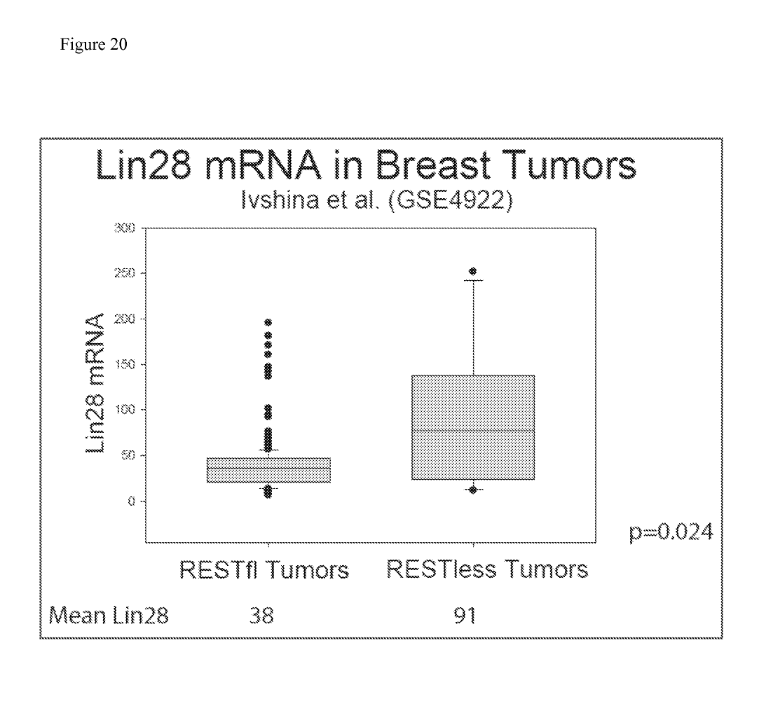

Analysis of publicly available cDNA microarray data from 289 human breast tumors showed that the median expression level of LIN28 in RESTless tumors was greater than the 90^{th} percentile expression in REST-containing (RESTfl) tumors (p=0.024) (FIG. 20). Furthermore, while RESTless tumors in mice showed local invasion into adjacent muscle tissue, in human patients, RESTless tumors show an increased lymph node metastasis relative to their REST-containing counterparts (Wagoner et al., 2010, PLoS Genet, 6: e1000979).

### Example 9

### REST4 Splicing: REST Regulation and the Role of PTB

To test the hypothesis that REST regulates REST4 splicing, cell lines stably expressing shRNA targeting either REST (shREST) or a non-targeting control (shControl) shRNA were generated. All cells were grown in 5% CO₂ at 37° C. HEK-293 and MCF7 cells were grown in DMEM with 4.5 g/L glucose, 2 mM L-Glutamine, and 10% fetal bovine serum from HyClone (Logan, Utah). T47D cells were grown in RPMI with L-glutamine, 10 ug/mL insulin, and 10% fetal bovine serum.

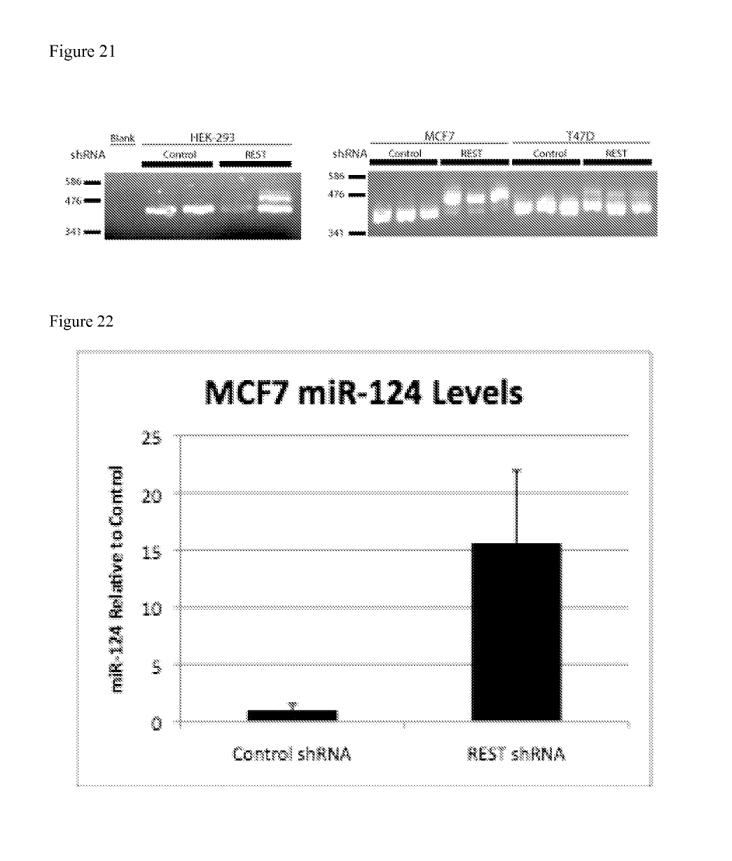

Analysis of REST splicing using primers flanking the excluded REST4 N-exon demonstrated that REST knockdown was sufficient to induce inclusion of the alternative exon within the REST coding region in HEK, T47D and MCF7 cells (FIG. 21A). Notably, no such alternative splicing was observed in control cells.

In addition to REST4 expression in REST knockdown MCF7 cells, heightened expression of the neuronal microRNA and REST target, miR-124 was also observed (FIG. 22). miR-124 levels were determined by quantitative real-time PCR analysis (qPCR) of REST4 performed using a cDNA template generated using the Invitrogen Superscript III reverse transcription system according to the manufacturer's directions. The qPCR mix used was the SYBR qRT-PCR System (Takara) and hREST4 Forward and hREST SV Region Reverse primers were amplified over 35 cycles. Eppendorf Triple Master Polymerase was used to amplify REST using SV+/−primers according to the manufacturer's instructions. Primers used to amplify the exon junctions surrounding introns 1 and 2: hREST SV region forward: (SEQ ID NO: 393, GAGCGAGTATCACTGGAGGAAACATTT). hREST SV region reverse: (SEQ ID NO: 394, ATAGTCACATACAGGGCAATTGAACTGC). Primers used to amplify REST4: hREST4 forward (Used with hREST SV reverse): (SEQ ID NO: 395, CATTCAGTGGGGTATGGATACC) and hREST4 reverse (Used with hREST SV forward): (SEQ ID NO:396, GCTTCTCACCCATCTAGATCAC). Taqman Kit #TM2197 has-miR-124# was used to detect the presence of mature, processed miR-124 according to the manufacturer's instructions.

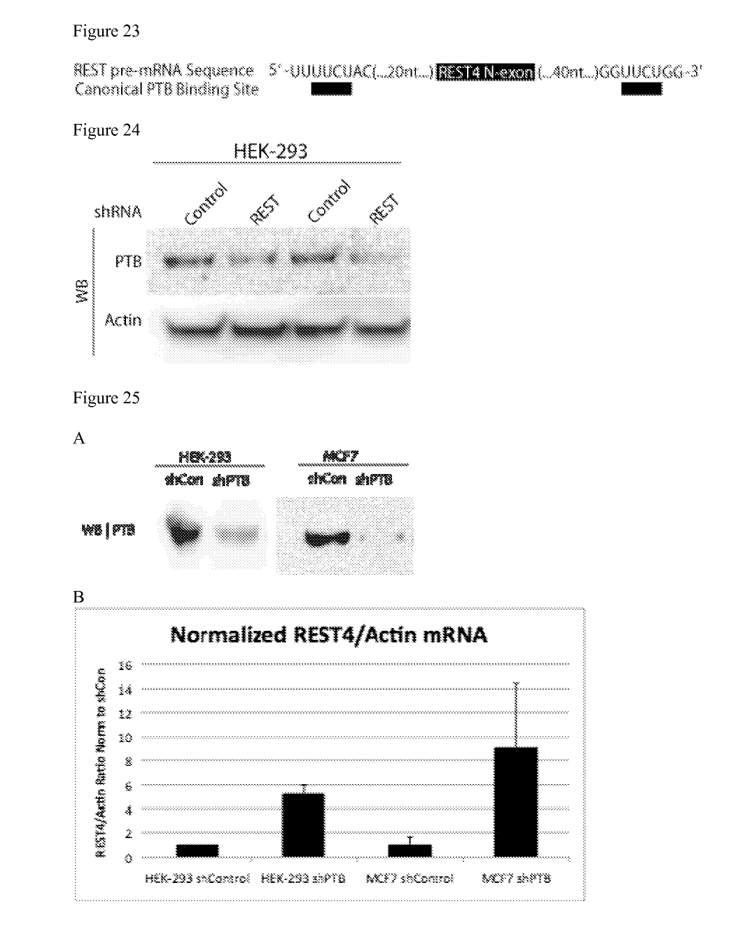

As miR-124 was known to regulate polypyrimidine tract binding protein (PTB) expression, and PTB is a repressor of alternative exon inclusion, it was hypothesized that PTB may be involved in regulating N-exon inclusion in REST4 splicing. Two canonical PTB binding sites 5′ and 3′ of the REST N-exon were identified (as shown in FIG. 23). If REST regulated its own splicing via miR-124, REST knockdown should have resulted in a downregulation of PTB protein and decreased binding of PTB to the proposed regulatory regions surrounding the N-exon (Chen et al., 2009, Nat Rev Mol Cell Biol, 10:741-754). This hypothesis was tested by Western blot analysis. Briefly, protein lysates were harvested in Triton lysis buffer with Sigma mammalian protease inhibitor cocktail P8340, sonicated and cleared by centrifugation at 15,000 rpm for 15 minutes. Protein gel electrophoresis (4-20% Tris-Glycine) was performed under conditions of 35 mA for 40 minutes, and thereafter proteins transferred onto PVDF at 23 V overnight. Membranes were blocked against non-specific hybridization using a 5% milk solution used to block and blot the membrane with antibodies to REST (purchased from Millipore, Billireca, Mass., Catalog. #07-0579), PTB antibody (purchased from Abcam, Cambridge, Mass., Catalog. #ab58131), or HRP-HA (obtained from Santa Cruz Biotechnology, Santa Cruz, Calif., Catalog. #sc7392). Decreased PTB protein levels were observed in HEK cells upon REST knockdown was observed (result shown in FIG. 24).

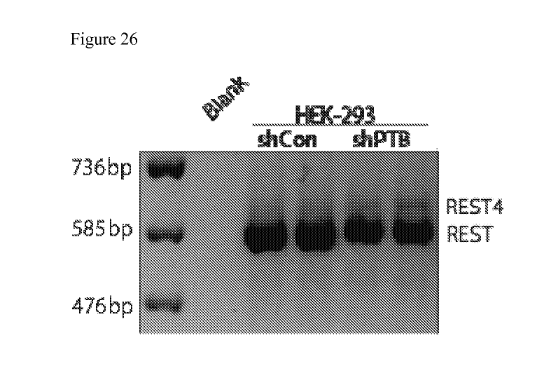

To determine whether loss of PTB was sufficient to induceREST4 splicing, stable HEK293 and MCF7 PTB knockdown (shPTB) and control cells were generated. REST4 mRNA was increased in shPTB HEK293 and MCF7 cells relative to shControl cells, suggesting that the observed loss of PTB protein may contribute to the alternative splicing (illustrated in FIG. 25). However, the observed increase of REST4 expression in HEK-293 cells expressing PTB shRNA was not sufficient to induce a large shift in the REST:REST4 ratio seen using primers that flank the N-exon (FIG. 26). These results suggested that though PTB may indeed be a repressor of N-exon inclusion, loss of PTB function cannot completely account for the increased expression of REST4 observed with REST knockdown in multiple cell lines. These data are consistent with the knowledge that small alternative exons are inefficiently recognized by splicing machinery, and that de-repression alone is not sufficient to induce cell type-specific splicing (Charlet et al., 2002, Mol Cell, 9:649-658). Rather, it is often the combination of a loss of a splicing repressor and the presence of a splicing enhancer that drives the inclusion of alternate exons.

The Examples above provide novel studies regarding the self-regulation of REST function by REST4 splicing, including the presence of the neural-specific microRNA miR-124 in breast cancer cell lines that lack REST function. Prior to these studies, no role for miR-124 outside the nervous system has been previously described. Thus miR-124 may play a key role in the neural-specific splicing observed in certain aggressive breast cancers.

### Example 10

### REST Regulates CELF Family Splicing Factors

To expand the understanding of the splicing factors at play in REST knockdown cell lines, DNA microarray analysis of mRNA from MCF7 shREST and shControl breast cancer cells was performed as described. Stable REST knockdown in HEK-293, T47D and MCF7 cells for microarray analysis was achieved using a Dharmacon SMARTvector lentiviral shRNA delivery system according to the manufacturer's instructions. Briefly, cells were infected in the presence of 8 mg/mL polybrene at an MOI of 5 with virus expressing a non-targeting control or REST shRNA. Puromycin selection was begun 48 hours after infection and maintained during cell expansion and experimentation. SMARTvector Lentiviral Particles (catalog #SH-042194-01-25) towards REST targeted the sequence GCAAACACCTCAATCGCCA (SEQ ID NO: 397), Non-Targeting SMARTvector shRNA Lentiviral particles (catalog #S-005000-01) were used as an infection control. PTB shRNA lentiviral construct was purchased from Open Biosystems (Huntsville, Ala.) catalog number TRCN0000001063.

HA-tagged lentiviral overexpression constructs were generated from the pSin-EF2-Lin28 plasmid. EcoRI and SpeI digest removed Lin28, which was replaced with an EcoRIx-Met-HA-tag-EcoRI-SpeI insert, where EcoRIx is the EcoRI overhang without the sixth nucleotide of the EcoRI cut site, preventing its digestion. Primers used for this purpose are listed: EcoRx-fMet-HA Tag: (SEQ ID NO: 398, AATTGATGTACCCATACGATGTTCCAGATTACGCTGAATTCATCGATA); and SpeI-ClaI-EcoRl-gaT-AH: (SEQ ID NO: 399, CTAGTATCGATGAATTCAGCGTAATCTGGAACATCGTATGGGTACATC). EcoRI and SpeI forward and reverse primers were used to clone mouse CELF4 and CELF6 coding sequence into the resulting vector.

For microarray data generation and processing, RNA was extracted using TRIzol (Invitrogen) according to the manufacturer's instructions from four independent plates of each cell line T47D, HEK-293 and MCF7, with two biological replicates of HEK-293 and T47D, and three biological replicates cells expressing REST shRNA and another two biological replicates expressing a non-targeting control shRNA.

All RNA reverse transcription, amplification and hybridizations were performed as set forth herein. RNA integrity and quality were assessed by comparing 28S/18S rRNA ratio using Agilent RNANano6000 chips on an Agilent 2100 Bioanalyzer. First and second strand cDNA synthesis steps, followed by in vitro transcription, were performed using the Ambion Amino Allyl Messageamp II kit. Cy3 and Cy5 (Amersham) dyes were coupled to the aRNA, with each fluorophore labeling a separate biological replicate, before fragmentation and dual hybridization to Nimblegen HG18 60 mer 385k Gene Expression Arrays (Nimblegen, Cat #A4542-00-01). For dual hybridization, shControl and shREST samples from the same cell line were competitively hybridized. Arrays were scanned on an Axon4000B and gene expression data was extracted, and RMA normalized using software provided by Nimblegen. All bioinformatic analyses were performed using MultiExperiment Viewer v4.6 (Saeed, Bhagabati et al. 2006). Two-class unpaired SAM Analysis was performed using MeV 4.6, and the delta value of 8.170, yielding <1% median false discovery rate.

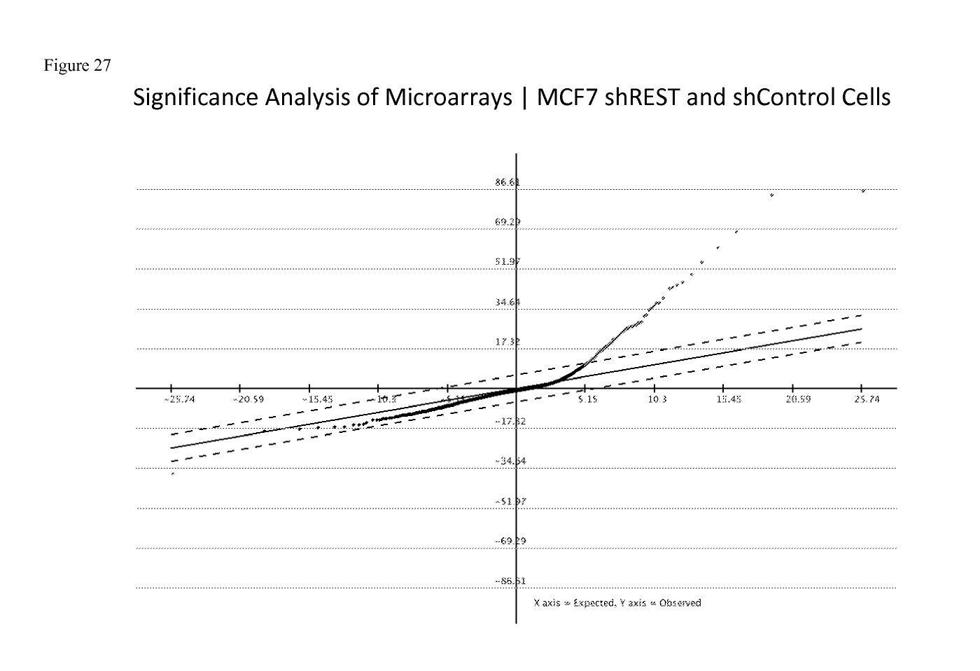

Following gene and sample normalization, significance analysis of microarrays was performed to detect genes that were differentially expressed upon REST knockdown (FIG. 27, median false discovery rate <1%). Consistent with the role of REST as a repressor, all of the RNA expression changes observed upon REST knockdown were upregulation events. In all, 118 mRNAs were upregulated upon REST knockdown in MCF7 cells. A series of concentric filters was applied to the 118 upregulated mRNAs to determine which were most likely to be directly involved in the regulation of REST4 splicing. First, microarray data was analyzed from the three cell lines that demonstrated REST4 splicing upon REST knockdown, HEK-293, T47D, and MCF7s with a focus on those genes that were upregulated with REST knockdown in all three lines. The list of gene candidates was further narrowed by selecting genes with known roles in exon inclusion, with particular emphasis on sequence-specific neural splicing factors, such as nPTB and Hu/Elav, as well as NOVA1 and NOVA2 and CELF family members. Of those genes identified, only those genes that had predicted REST binding elements were examined.

TABLE 6
Genes Upregulated in the Absense of Functional REST

| | | Transcript | SEQ ID |
|---|---|---|---|
| Name | Abbreviation | Accession No. | NO |
| Homo sapiens CUGBP, Elav- | CELF4 | NM_020180 | SEQ ID |
| like family member 4 | | | NO: 400 |
| Homo sapiens CUGBP, Elav- | CELF5 | NM_021938 | SEQ ID |
| like family member 5 | | | NO: 401 |
| Homo sapiens CUGBP, Elav- | CELF6 | NM_052840 | SEQ ID |
| like family member 6 | | | NO: 402 |

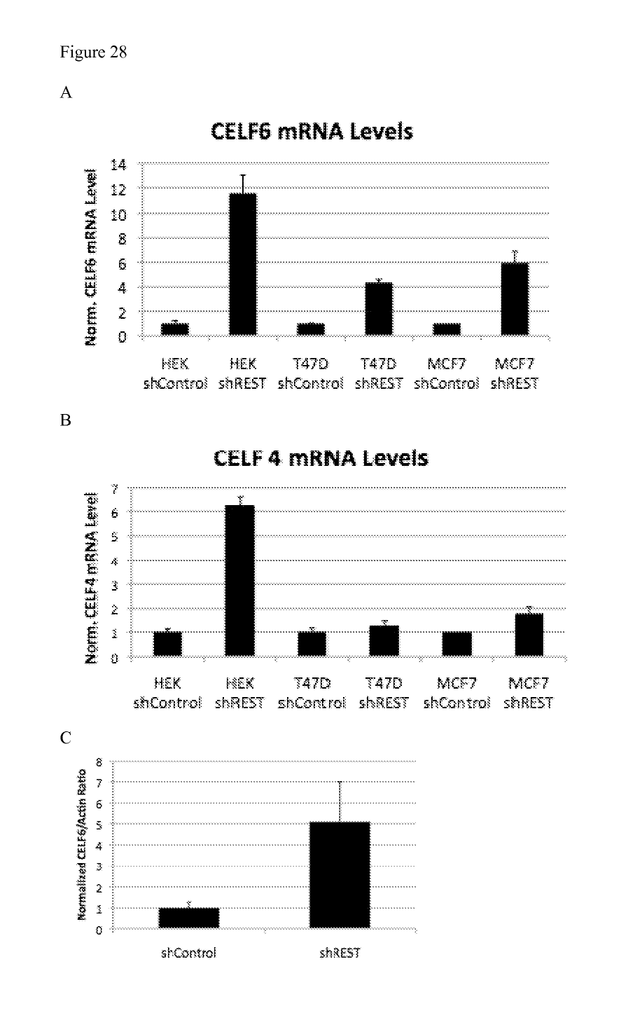

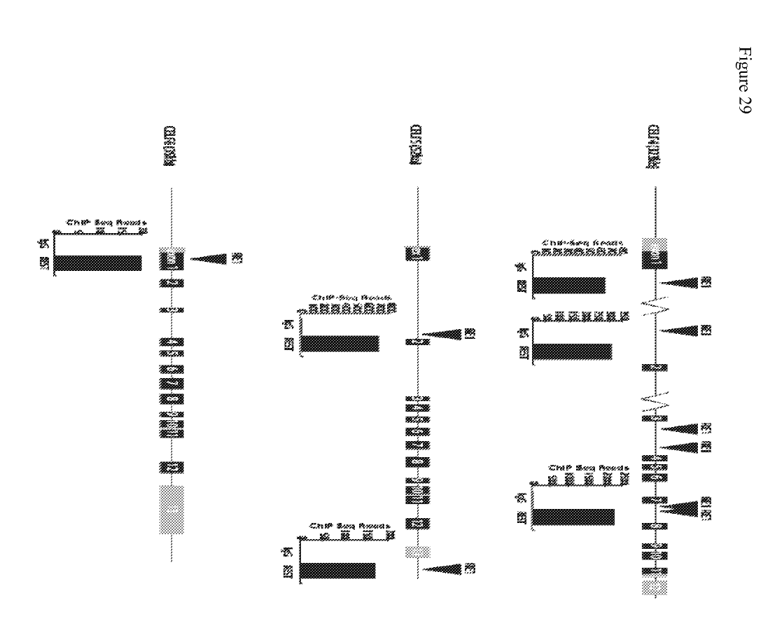

CELF6 was the only gene to meet all of the above criteria, including being overexpressed at least 4-fold upon REST knockdown in three independent cell lines (FIG. 28). CELF6 is expressed predominantly in kidney, testes, and brain and it directly binds RNA elements surrounding small exons in pre-mRNA, promoting their inclusion (Ladd et al., 2004, J Biol Chem, 279:17756-17764). Importantly, CELF6 contains a consensus RE1-site, indicating that it is a potential REST target gene (FIG. 28). Publicly available REST ChIP-Seq data suggested that REST strongly binds this CELF6 RE1 site in Jurkat T-cells (FIG. 29) (Johnson et al., 2007, Science, 316:1497-1502). Interestingly, the CELF6 homolog CELF4 was also upregulated more than two-fold in HEK-293 and MCF7 cells upon REST knockdown (FIG. 28), contained six consensus RE-1 sites, and was also identified as a REST target in the REST ChIP-Seq experiment (FIG. 29). Furthermore, CELF5 was also elevated two-fold upon REST knockdown in HEK-293 cells, contains two RE1 sites, and was identified as a REST-bound gene in the ChIP-Seq database (FIG. 29). Importantly, all of the RE1 sites found in these CELF genes were highly conserved between human, mouse, and rat genomes (UCSC Genome browser, data not shown). Together, these data suggest that multiple CELF family members may be directly regulated by REST function.

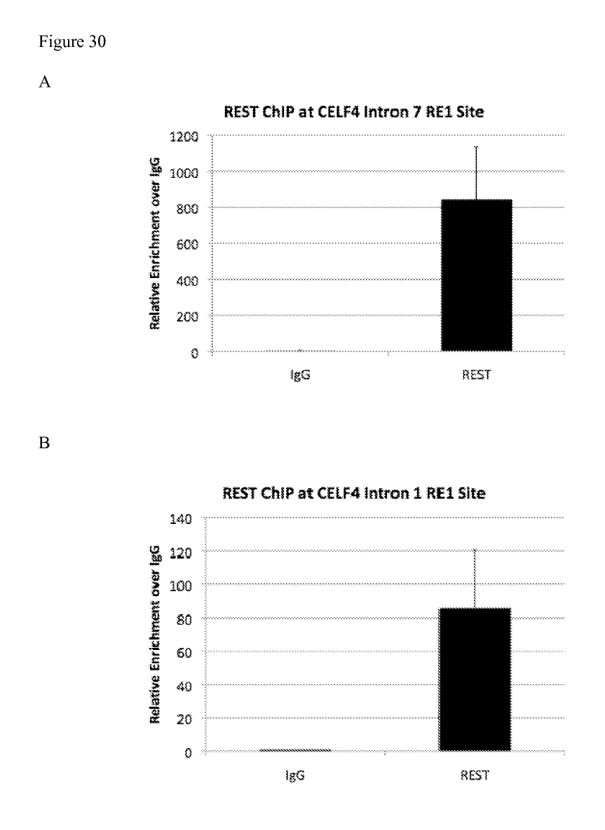

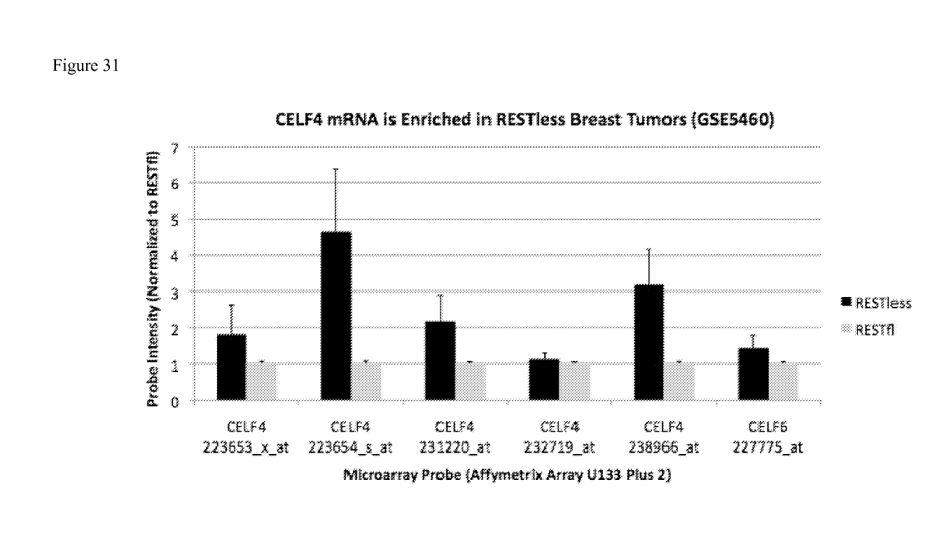

To verify the findings of the ChIP-Seq experiment, REST ChIP qPCR experiments were performed with chromatin from MCF7 cells to examine REST binding at the strongest and the weakest RE1 sites in CELF4, as predicted by ChIP-Seq read frequency. REST ChIP followed by qPCR showed 80-fold and 800-fold enrichment for REST immunoprecipitation over IgG at the first RE1 site in CELF4 intron 1 and the double RE1 site in intron 7, respectively (FIG. 30). The RE1 site located in intron 7 contains two consensus REST binding elements sites separated by six nucleotides. RE1 sites located so close together often show synergistic binding, which likely accounts for the strong affinity observed at those elements. Importantly, CELF4 mRNA levels were upregulated in breast tumors with low REST function (RESTless) with respect to their normal, RESTfl, counterparts (FIG. 31). These data identified CELF4 as a likely REST target gene, and its heightened mRNA level in RESTless tumors was likely due to the lower REST function in these cells.

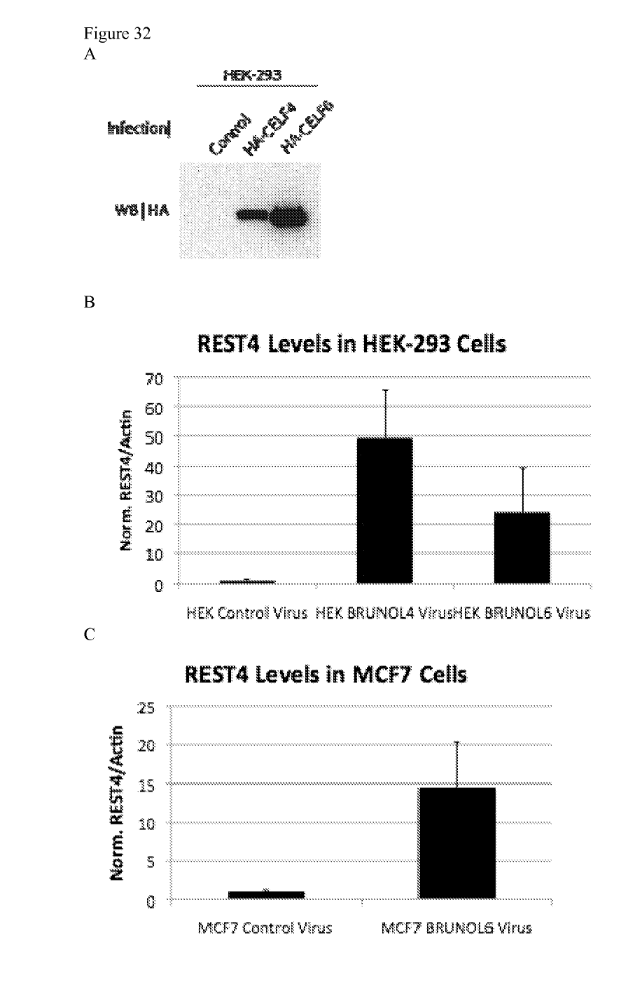

Overexpression of either CELF4 or CELF6 resulted in a dramatic shift in REST splicing in multiple cell systems (FIG. 32). Expression of HA-tagged CELF6 resulted in 15-fold and 24-fold increases in REST4 levels in MCF7 and HEK-293 cells, respectively. Similarly, expression of HA-CELF4 in HEK-293 cells resulted in a 49-fold increase in REST4 levels. These data demonstrate that overexpression of CELF4 and CELF6 was sufficient to induce REST4 splicing, indicating that their expression in RESTless tumors may contribute to the heightened levels of REST4.

Prior to these studies, little work has been done investigating the signaling pathways surrounding REST4 splicing, and to date, no splicing factors have been directly linked to the alternative variant. The present studies identify one likely repressor of REST4 splicing, PTB. In two different cell systems generated herein it is shown that knockdown of PTB is sufficient to induce a moderate increase in REST4 splicing.

These studies suggest that REST regulates the expression of multiple CELF family members, including CELF6, CELF4, and possibly CELF5. All three of these family members are closely related to one another, and are, in many senses, functionally redundant (Barreau et al., 2006, Biochimie, 88:515-525). CELF4-6 all have the ability to enhance the inclusion of the cTNT exon 5, and CELF4 and CELF6 have also been shown to regulate exon 11 exclusion in the insulin receptor (Barreau et al., 2006, Biochimie, 88:515-525). Here it is shown that overexpression of CELF4 and CELF6 is sufficient to drive REST4 splicing in vitro.

PTB and CELF-family splicing factors are known to dynamically antagonize one another in the regulation of multiple genes, including cTNT. Given that PTB knockdown and CELF4/6 overexpression both upregulate REST4 levels in multiple cell systems, it is predicted that similar antagonistic regulation of the N-exon may exist. These studies suggest PTB, CELF4 and CELF6 as a potential regulators of N-exon inclusion in REST mRNA processing. Intriguingly, it was found that positive and negative effectors of N-exon inclusion are themselves regulated by REST function. Paradoxically, the result of this is that REST functionally regulates its own splicing, which in turn regulates REST function, creating an interesting feed-forward loop that likely plays a critical role in aggressive breast cancer.

In addition, the invention is not intended to be limited to the disclosed embodiments of the invention. It should be understood that the foregoing disclosure emphasizes certain specific embodiments of the invention and that all modifications or alternatives equivalent thereto are within the spirit and scope of the invention as set forth in the appended claims.

We claim:

1. A method for identifying a patient with breast cancer having a reduced disease-free survival time, the method comprising:
(a) assaying a tumor sample from the patient for expression of one or a plurality of genes selected from the genes contained in Tables 1 or 3;
(b) detecting differential expression of one or a plurality of the genes contained assayed in step (a);
(c) identifying a patient with reduced disease-free survival, wherein differential expression one or a plurality of said gene or genes is detected in step (b).

2. The method of claim 1, wherein the assay of step (a) comprises treating the tumor sample to prepare biomolecules from said genes comprising mRNA, cDNA or protein, wherein said prepared biomolecules are capable of being detected or contacted by a reagent used in said assay and thereby detected.

3. The method of claim 1 wherein one or a plurality of genes further comprise those contained in Tables 2, 4, or 6.

4. The method of claim 1, wherein a plurality of genes detected are Adaptor-related protein complex 3, beta 2 subunit; Bassoon (presynaptic cytomatrix protein); Complexin 1; Complexin 2; Dispatched homolog 2 (Drosophila); Golgi Autoantigen 7B; Hemoglobin alpha 2; Potassium voltage-gated channel Shab-related subfamily member 1; Mitogen-activated protein kinase 8 interacting protein 2; Matrix metallopeptidase 24 (membrane-inserted); PiggyBac transposable element derived 5; RGD motif, leucine rich repeats, tropomodulin domain and proline-rich containing; Reticulon 2; RUN domain containing 3A; Secretory carrier membrane protein 5; Synaptosomal-associated protein, 25kDa; Stathmin-like 3; Transmembrane protein 145; Transmembrane protein 198; or VGF nerve growth factor inducible.

5. The methods of claim 1, 3 or 4 wherein a plurality of genes are detected.

6. The methods of claim 1, 3 or 4 wherein said differential expression is elevated gene expression.

7. The methods of claim 1, 3 or 4 wherein the cancer is estrogen receptor positive breast cancer.

8. The methods of claim 1, 3 or 4 wherein the cancer is estrogen receptor negative breast cancer.

9. The method of claim 1, wherein the plurality of genes detected comprise LIN28 or CELF4, CELF5, or CELF6.

10. The method of claim 9, wherein the genes are assayed by microarray, reverse transcriptase-polymerase chain reaction assay (RT-PCR), quantitative RT-PCR (qRT-PCR), real-time polymerase chain reaction assay (RT-RTPCR), or immunoassay or immunohistochemical assay.

11. A method for identifying a patient with breast cancer having a reduced disease-free survival time, the method comprising:
(a) assaying a tumor sample from the patient for altered or reduced expression of RE1 Silencing Transcription Factor/Neuron restrictive silencing factor (REST/NRSF);
(b) detecting altered or reduced expression of REST/NRSF assayed in step (a);
(c) identifying a patient with reduced disease-free survival, wherein REST/NRSF expression is altered or reduced as detected in step (b).

12. The method of claim 11, wherein the assay of step (a) comprises treating the tumor sample to prepare a REST/NRSF biomolecule from said genes comprising mRNA, cDNA or protein, wherein said prepared biomolecules are capable of being detected or contacted by a reagent used in said assay and thereby detected.

13. The method of claim 11, wherein the cancer is estrogen receptor positive breast cancer.

14. The method of claim 11, wherein the cancer is estrogen receptor negative breast cancer.

15. The method of claim 11, wherein reduced protein expression of REST/NRSF is detected.

16. The method of claim 11, wherein altered protein expression is detected.

17. The method of claim 16, wherein the altered protein expression is REST4 splice variant.

18. The methods of claim 1 or 3, wherein mRNA of the genes in Table 1, 2, 3, 4, or 6 is isolated and assayed to determine gene expression levels.

19. The methods of claim 1 or 3 wherein protein products of the genes in Table 1, 2, 3, 4, or 6 are isolated and assayed to determine gene expression levels.

20. The methods of claim 18, wherein mRNA is assayed by microarray, reverse transcriptase-polymerase chain reaction assay (RT-PCR), reverse transcriptase-polymerase chain reaction assay (qRT-PCR), or real-time reverse transcriptase-polymerase chain reaction assay (RT-RTPCR).

21. The method of claim 19 wherein protein is assayed by immunoassay or immunohistochemical assay.

22. The method of claim 11, wherein REST/NRSF mRNA or REST4 mRNA is assayed to determine gene expression levels.

23. The method of claim 11, wherein protein products of REST/NRSF or REST4 are assayed to determine gene expression levels.

24. The method of claim 22, wherein REST/NRSF mRNA is assayed by reverse transcriptase-polymerase chain reaction assay (RT-PCR), reverse transcriptase-polymerase chain reaction assay (qRT-PCR), or real-time reverse transcriptase-polymerase chain reaction assay (RT-RTPCR).

25. The method of claim 23, wherein protein is assayed by immunoassay or immunohistochemical assay.

26. The method of claim 25, wherein said immunoassay or immunohistochemical assay is performed using an antibody immunologically specific for a DNA binding domain of REST/NRSF protein.

27. The method of claim 26, wherein the antibody is immunologically specific for the C-terminal DNA binding domain of REST/NRSF protein.

28. A method for identifying a patient with breast cancer having a reduced disease-free survival time, the method comprising:
(a) assaying a tumor sample from the patient for expression of miR-124;
(b) detecting the presence miR-124 in the sample assayed in step (a);
(c) identifying a patient with reduced disease-free survival, wherein miR-124 is detected in step (b).

29. The method of claim 28, wherein the tumor sample is treated to prepare a biomolecule from said miR-124 comprising mRNA or cDNA prepared therefrom, wherein said prepared biomolecule is capable of being detected or contacted by a reagent used in said assay and thereby detected.

30. The method of claim 1, 11 or 28, wherein a portion of the tumor sample is substantially consumed in said assay.

31. A kit for diagnosing or prognosing reduced disease-free survival time in a human with cancer, the kit comprising a plurality of nucleotide primers that each specifically hybridize to one or a plurality of the genes identified in Table 1, 3, or 6.

32. A kit for diagnosing or prognosing reduced disease-free survival time in a human with cancer, the kit comprising a plurality of nucleotide primers that each specifically hybridize to REST4 or mir-124.

33. A kit for diagnosing or prognosing reduced disease-free survival time in a human with cancer, the kit comprising a plurality of antibodies that each specifically bind to a protein produced by expression of one or a plurality of the genes identified in Table 1, 3, or 6.

34. A kit for diagnosing or prognosing reduced disease-free survival time in a human with cancer, the kit comprising an antibody specific for the C-terminus of REST/NRSF protein.
